(19) United States
(12) Patent Application Publication
(10) Pub. No.: US 2026/0053921 A1
(43) Pub. Date: Feb. 26, 2026

(54) A MULTIPLEXED RNA REGULATION PLATFORM FOR PRIMARY IMMUNE CELL ENGINEERING

(71) Applicant: The Board of Trustees of the Leland Stanford Junior University, Stanford, CA (US)

(72) Inventors: Lei S. Qi; Victor Tieu, Stanford, CA (US); Crystal L. Mackall, Stanford, CA (US); Elena Sotillo

(73) Assignee: The Board of Trustees of the Leland Stanford Junior University, Stanford, CA (US)

(21) Appl. No.: 19/102,637

(22) PCT Filed: Aug. 24, 2023
(86) PCT No.: PCT/US2023/072798

§ 371 Date: Feb. 10, 2025

Related U.S. Application Data
(60) Provisional application No. 63/400,578, filed on Aug. 24, 2022.

(51) Int. Cl. A61K 40/11; A61K 40/31; A61K 40/42; A61P 35/00; C07K 14/725; C07K 16/30; C12N 9/22; C12N 15/11; C12N 15/90

(52) U.S. Cl. CPC A61K 40/11; A61K 40/31; A61K 40/4258; A61P 35/00; C07K 14/7051; C07K 16/3084; C12N 9/226; C12N 15/11; C12N 15/907; C12N 2310/20

(57) ABSTRACT

The present disclosure provides a versatile and multi-functional platform for transcriptome regulation using the RNA-guided. RNA-targeting activity of type VI-D CRISPR effectors with RNA-guided RNA endonuclease activity combined with guide arrays that express a plurality of guide RNAs. The system can be used to perform quantitative, reversible, and massively-multiplexed gene knockdown in primary human T cells and to perform multiplexed suppression of exhaustion-associated genes in T cells. The system can be used to enhance the anti-tumor activity of dysfunctional CAR T cells.

### CROSS-REFERENCE TO RELATED APPLICATIONS

This application claims priority to U.S. Provisional Application No. 63/400,578, filed Aug. 24, 2022, the disclosure of which is herein incorporated by reference in its entirety for all purposes.

### STATEMENT AS TO RIGHTS TO INVENTIONS MADE UNDER FEDERALLY SPONSORED RESEARCH AND DEVELOPMENT

This invention was made with Government support under Grant No. 1R21CA270609-01 awarded by the National Cancer Institute. The Government has certain rights in this invention.

### BACKGROUND

Adoptive T cell therapy, including Chimeric Antigen Receptor (CAR) T cell therapy, has revolutionized the treatment of refractory B cell and plasma cell malignancies in patients and has demonstrated promising curative potential in other aggressive preclinical cancer models. Despite these successes, however, significant barriers to progress remain, including a high rate of primary and acquired resistance, with very limited clinical efficacy against solid cancers¹⁻⁵. Recent studies have implicated T cell intrinsic mechanisms of dysfunction such as T cell exhaustion and metabolic dysregulation in these failure modes^{1, 6-10.}

Born of concurrent advances in CRISPR/Cas9-based methods and technologies, next-generation genetically engineered CAR T cells offer a potential solution to these challenges, as well as an opportunity to create new therapeutic modalities^{3, 11}. For example, Cas9 can be used to introduce pooled systematic genetic perturbations, often at genome-wide scale, in primary human T cells to screen for negative regulators of T cell activity in various cancer-related contexts¹²⁻¹⁹. Screen hits can then be specifically deleted by Cas9 knockout or base-editing strategies to prevent dysfunction or augment anti-tumor activity, including T cell expansion and persistence, solid tumor infiltration, cytokine secretion, and cytotoxicity^{12-16,19-24} Alternatively, components of donor-specific proteins such as TRAC (TCR) and B2M (HLA) can be knocked out to generate allogeneic “off-the-shelf” T cells that do not trigger immune rejection or graft-versus-host disease^{3,11,25-28}.

Although the intersection of CRISPR engineering and adoptive T cell therapy holds great promise for the future of cancer treatment, Cas9-based systems are limited in their safety and efficacy, as well as in their potential for broader applications. Both Cas9 nuclease and base-editors introduce binary and irreversible changes to the T cell genome^{29,30}. These methods completely ablate gene function, which may adversely affect T cell fitness and activity^{21,31-35}, and prevent such perturbations from being dynamically regulated. Furthermore, Cas9 nuclease is incompatible with safe and efficient large-scale multiplexing of gene knockouts—the induction of multiple double-strand breaks (DSB) can cause chromosomal translocation/truncation^{27,36,37} and p53-related genotoxicity^{11.38}, which raises the possibility of malignant transformation. While Cas9 base-editors mitigate DSB-related risks by nicking genomic DNA, the trade-off between on-target editing efficiency and broad off-target editing by promiscuous deaminase activity limits large-scale multiplexing^{3,27,39-41}. Taken altogether, Cas9-based tools are wholly unable to perturb complex multi-gene programs (such as those driving T cell metabolism, signaling, and exhaustion) safely and effectively, which is likely to be necessary for engineering optimized, next generation adoptive T cell therapies.

To overcome the limitations posed by current state-of-the-art gene editing technologies in primary human T cells, the present disclosure provides MEGA (Multiplexed Effector Guide Arrays), a multi-functional platform for transcriptome regulation using CRISPR/Cas13d. Cas13d is a recently-described, compact class 2 type VI-D CRISPR effector with RNA-guided RNA endonuclease activity^{42,43}. Unlike Cas9, Cas13d does not bind, target, or cut DNA⁴². Rather, it complexes with a CRISPR-associated RNA (crRNA) containing a programmable ˜23 nt spacer that directs the ribonucleoprotein to sequence-specific RNA transcripts for targeted degradation without PAM restriction. Additionally, Cas13d has the unique ability to process poly-crRNA guide arrays into individual crRNAs to facilitate efficient simultaneous targeting of multiple RNA transcripts in single cells^{42,44}. Finally, Cas13d is roughly two-thirds the size of wild-type Cas9 (fusion Cas9 variants are even larger), making it highly amenable to T cell manufacturing.

The present disclosure describes compositions and methods for massively-multiplexed, quantitative, and reversible perturbation of the cellular transcriptome. To tackle established unmet needs, the present disclosure describes deployment of MEGA across a broad swath of applications ranging from the modulation of CAR signal transduction to the enhancement of anti-tumor activity in dysfunctional high affinity GD2-targeting (HA-28z) CAR T cells, a well-characterized in vitro model that recapitulates key molecular, phenotypic, and functional features of T cell exhaustion^{7,45-47}. The present disclosure also describes leveraging MEGA to target distinct gene-sets implicated in metabolic reprogramming and disrupt an entire metabolic pathway to alleviate adenosine-mediated CAR T cell immunosuppression.

### BRIEF SUMMARY

The present disclosure provides compositions comprising genetically modified T cells that express (i) a class 2 type VI-D CRISPR effector having RNA-guided RNA endonuclease activity; and (ii) a guide array comprising a plurality of CRISPR-associated RNA (crRNA) molecules, systems for multiplexed transcriptomic regulation, fusion proteins comprising a class 2 type VI-D CRISPR effector having RNA-guided RNA endonuclease activity a destabilization domain (DD), nucleic acids such as plasmids and vectors that encode a class 2 type VI-D CRISPR effector having RNA-guided RNA endonuclease activity, a guide array, a CAR, and/or a fusion protein of the disclosure, and guide arrays comprising nucleic acids (polynucleotides) comprising a direct repeat sequence and a spacer sequence. The disclosure also provides methods for producing a modified T cell that expresses a class 2 type VI-D CRISPR effector and a guide array of the disclosure, methods for regulating gene expression in a T cell, method for screening to identify regulators of T cell activity, methods for increasing the proliferation of a T cell, methods for treating cancer or a tumor in a subject or patient in need of treatment, and methods for increasing the anti-tumor activity of a T cell.

In one aspect, the disclosure provides a genetically modified T cell comprising:
(i) a class 2 type VI-D CRISPR effector having RNA-guided RNA endonuclease activity; and
(ii) a guide array comprising a plurality of CRISPR-associated RNA (crRNA) molecules, wherein the crRNA molecules independently comprise a direct repeat sequence and a spacer sequence that binds a target RNA, and wherein the crRNA molecules bind to different target mRNAs or different regions of the same target mRNA.

In some embodiments, the genetically modified T cell comprises a nucleic acid encoding (i), a nucleic acid encoding (ii), or a nucleic acid encoding both (i) and (ii), wherein the nucleic acids encoding (i) or (ii), or both (i) and (ii), are stably integrated into the genome of the T cell.

In some embodiments, the class 2 type VI-D CRISPR effector having RNA-guided RNA endonuclease activity comprises a fusion protein comprising a destabilization domain (DD).

In some embodiments, the class 2 type VI-D CRISPR effector having RNA-guided RNA endonuclease activity is a Cas13 nuclease selected from the group consisting of Cas13a, Cas13b, 13bt1, Cas13bt2, Cas13c, Cas13d, RfxCas13d, Cas13e, and functional variants thereof.

In some embodiments, the guide array is a multicistronic array comprising the plurality of crRNA molecules. In some embodiments, the guide array comprises from 2 to 10 crRNA molecules. In some embodiments, the guide array comprises greater than 10 crRNA molecules.

In some embodiments, the guide array comprises crRNA molecules that bind to an mRNA encoding a protein associated with T cell exhaustion. In some embodiments, the protein associated with T cell exhaustion is selected from the group consisting of TOX, TOX2, NR4A1, NR4A2, NR4A3, TET2, IRF4, JUNB, BATF3, DHX37, FLI1, ZC3H12A, SOCS1, TCEB2, PDCD1 (PD-1), HAVCR2 (TIM3), LAG3, CTLA4, TIGIT, FAS, TRAC, CBLB, RASA2, PTPN2, CD5, ENTPD1, CD46, B2M, and combinations thereof.

In some embodiments, the T cell or the primary T cell further comprises a chimeric antigen receptor (CAR). In some embodiments, the CAR binds to an antigen expressed by a tumor. In some embodiments, the CAR binds to an antigen selected from the group consisting of Her-2, B7-H3, GPC2, GD2, CD19, CD20, CD22, MAGE, BAGE, CAGE, GAGE, HAGE, LAGE, PAGE, PRAME, NY-ESO-1, NY-SEO-1, tyrosinase, Melan-A/MART, gp100, TRP-1, TRP-2, CD30, EGFR, EGFRvIII, FAP, CD33, CD123, PD-L1, IGF1R, CD4, CSPG4, B7-H4, NKG2D, CSI, CD138, EpCAM, EBNA3C, GPA7, CD244, CA-125, ETA, CEA, CD52, MUCSAC, c-Met, FAB, WT-1, PSMA, AFP, BCMA, Mesothelin, GPC3, MUC1 and CTAG1B.

In some embodiments, intracellular signaling by the CAR upregulates T cell exhaustion markers in control T cells. In some embodiments, the control T cell does not comprise (i) a class 2 type VI-D CRISPR effector having RNA-guided RNA endonuclease activity; and (ii) a guide array comprising a plurality of CRISPR-associated RNA (crRNA) molecules, wherein the crRNA molecules independently comprise a direct repeat sequence and a spacer sequence that binds a target RNA, and wherein the crRNA molecules bind to different target mRNAs or different regions of the same target mRNA.

In some embodiments, the exhaustion markers are selected from the group consisting of LAG3, PDCD1 (PD-1), HAVCR2 (TIM3), CTLA4 and ENTPD1 and combinations thereof.

In some embodiments, the guide array comprises crRNA molecules that bind to mRNA encoding LAG3, PDCD1 (PD-1), HAVCR2 (TIM3), CTLA4 and ENTPD1, or a combination thereof. In some embodiments, the guide array comprises crRNA molecules that bind to mRNA expressed by the CAR.

In some embodiments, the T cell is selected from the group consisting of a human T cell and a primary human T cell.

In another aspect, the disclosure provides a nucleic acid encoding (i) a class 2 type VI-D CRISPR effector having RNA-guided RNA endonuclease activity; and (ii) a guide array comprising a plurality of CRISPR-associated RNA (crRNA) molecules, wherein the crRNA molecules independently comprise a direct repeat sequence and a spacer sequence that binds a target RNA, and wherein the crRNA molecules bind to different target mRNAs or different regions of the same target mRNA. In some embodiments, the class 2 type VI-D CRISPR effector having RNA-guided RNA endonuclease activity is a Cas13 nuclease selected from the group consisting of Cas13a, Cas13b, 13bt1, Cas13bt2, Cas13c, Cas13d, RfxCas13d, Cas13e, and functional variants thereof. In some embodiments, the guide array is a multicistronic array comprising the plurality of crRNA moleculesIn some embodimentsthe guide array comprises from 2 to greater than or equal to 10 crRNA molecules. In some embodiments, the guide array comprises crRNA molecules that bind to mRNA encoding a protein associated with T cell exhaustion. In some embodiments, the protein associated with T cell exhaustion is selected from the group consisting of TOX, TOX2, NR4A1, NR4A2, NR4A3, TET2, IRF4, JUNB, BATF3, DHX37, FLI1, ZC3H12A, SOCS1, TCEB2, PDCD1 (PD-1), HAVCR2 (TIM3), LAG3, CTLA4, TIGIT, FAS, TRAC, CBLB, RASA2, PTPN2, CD5, ENTPD1, CD46, B2M, and combinations thereof.

In another aspect, the disclosure provides a system for multiplexed transcriptomic regulation, comprising: (i) an expression cassette comprising a nucleic acid sequence encoding a class 2 type VI-D CRISPR effector having RNA-guided RNA endonuclease activity; and (ii) an expression cassette comprising a nucleic acid sequence encoding a guide array comprising a plurality of crRNA molecules, wherein the crRNA molecules comprise a direct repeat sequence and a spacer sequence that binds a target mRNA, and wherein the crRNA molecules bind to different target mRNAs or different regions of the same target mRNA.

In some embodiments, the system further comprises (iii) a T cell or primary T cell.

In another aspect, the disclosure provides a method for producing a modified T cell, the method comprising: (i) transducing a T cell with an expression vector comprising a nucleic acid sequence encoding a class 2 type VI-D CRISPR effector having RNA-guided RNA endonuclease activity; and (ii) transducing the T cell with an expression vector comprising a nucleic acid sequence encoding a guide array comprising a plurality of CRISPR-associated RNA (crRNA) molecules, wherein the crRNA molecules independently comprise a direct repeat sequence and a spacer sequence that binds a target mRNA, and wherein the crRNA molecules bind to different target mRNAs or different regions of the same target mRNA, thereby producing the modified T cell.

In some embodiments, the method further comprises (iii) transducing the T cell with an expression vector comprising a nucleic acid sequence encoding a CAR.

In some embodiments of the method, step (i) is performed before step (ii); step (iii) is performed before step (ii), or steps (i) and (iii) are performed before step (ii).

In some embodiments, one or more of the expression vectors of steps (i), (ii) and/or (iii) are stably integrated into the genome of the T cell.

In some embodiments of the method, expression and intracellular signaling by the CAR upregulates T cell exhaustion markers in control T cells that do not comprise the expression vectors of steps (i) and (ii).

In another aspect, the disclosure provides a fusion protein comprising a class 2 type VI-D CRISPR effector having RNA-guided RNA endonuclease activity covalently linked to a destabilization domain (DD) polypeptide.

In some embodiments, the class 2 type VI-D CRISPR effector having RNA-guided RNA endonuclease activity is a Cas13 nuclease selected from the group consisting of Cas13a, Cas13b, 13bt1, Cas13bt2, Cas13c, Cas13d, RfxCas13d, Cas13e, and functional variants thereof.

In some embodiments, the DD comprises an E. coli dihydrofolate reductase DD linked to the C-terminus of the class 2 type VI-D CRISPR effector having RNA-guided RNA endonuclease activity.

In some embodiments, the disclosure provides a nucleic acid encoding a fusion protein described herein.

In another aspect, the disclosure provides a method for regulating gene expression in a T cell, the method comprising:
(a) transducing a T cell with (i) an expression vector comprising a nucleic acid sequence encoding a fusion protein described herein; and (ii) an expression vector comprising a nucleic acid sequence encoding a guide array comprising a crRNA molecule, wherein the crRNA molecule comprises a direct repeat sequence and a spacer sequence that binds a target mRNA expressed by a target gene, and
(b) contacting the T cell with a compound that binds to and stabilizes the DD, wherein expression of the target gene is decreased in the presence of the compound compared to expression of the target gene in the absence of the compound, thereby regulating gene expression in the T cell.

In some embodiments, the fusion protein is degraded in the T cell in the absence of the compound.

In some embodiments, expression of the target gene is increased after removal of the compound, thereby reversibly regulating expression of the target gene.

In some embodiments, the guide array comprises a plurality of crRNA molecules that bind to different target mRNAs or different regions of the same target mRNA.

In some embodiments, expression of the target gene(s) is regulated by the compound in a dose-dependent manner. In some embodiments, the compound is trimethoprim (TMP).

In another aspect, the disclosure provides a method for screening to identify regulators of T cell activity, the method comprising expressing in a T cell or population of T cells: (i) a class 2 type VI-D CRISPR effector having RNA-guided RNA endonuclease activity; and (ii) a library of guide arrays, where individual guide arrays comprise one or more CRISPR-associated RNA (crRNA) molecules, wherein the crRNA molecules comprise a direct repeat sequence and a spacer sequence that binds a target RNA, and wherein the one or more crRNA molecules bind to different target mRNAs or different regions of the same target mRNA; culturing the T cell(s) to produce a clonal population of expanded T cells; and determining if a guide array is enriched or depleted in the clonal population of expanded T cells, wherein if a guide array is enriched, then the target mRNA encodes a negative regulator of T cell activity, or if a guide array is depleted, then the target mRNA encodes a positive regulator of T cell activity.

In some embodiments, the T cell activity is T cell proliferation, increased cytokine secretion, or increased tumor cell killing.

In some embodiments, T cells comprising an enriched guide array have an effector memory phenotype, and T cells comprising a depleted guide array have a central memory or stem cell memory phenotype.

In some embodiments of the method, the individual guide arrays comprise crRNA molecules that bind to an mRNA encoding a protein associated with T cell exhaustion. In some embodiments, the protein associated with T cell exhaustion is selected from the group consisting of TOX, TOX2, NR4A1, NR4A2, NR4A3, TET2, IRF4, JUNB, BATF3, DHX37, FLI1, ZC3H12A, SOCS1, TCEB2, PDCD1 (PD-1), HAVCR2 (TIM3), LAG3, CTLA4, TIGIT, FAS, TRAC, CBLB, RASA2, PTPN2, CD5, ENTPD1, CD46, B2M, and combinations thereof.

In some embodiments, the library of guide arrays comprises one or more individual guide arrays comprising multicistronic arrays comprising a plurality of crRNA molecules. In some embodiments, the individual guide arrays comprise from 2 to 10 crRNA molecules. In some embodiments, the individual guide arrays comprise a pair of crRNA molecules.

In some embodiments, determining if a guide array is enriched or depleted in the clonal population of expanded T cells comprises sequencing the guide RNAs present in the T cells.

In another aspect, the disclosure provides a method for increasing proliferation of a T cell, the method comprising transducing a T cell with
(i) an expression vector comprising a nucleic acid sequence encoding a class 2 type VI-D CRISPR effector having RNA-guided RNA endonuclease activity; and
(ii) an expression vector comprising a nucleic acid sequence encoding a guide array comprising one or more crRNA molecules, wherein the crRNA molecules independently comprise a direct repeat sequence and a spacer sequence that binds a target mRNA, and wherein the crRNA molecules bind to different target mRNAs or different regions of the same target mRNA, wherein proliferation of the T cell is increased compared to a control T cell that expresses a non-targeting control guide array comprising one or more crRNA molecules that do not bind to a target mRNA in (ii).

In some embodiments, the method further comprises (iii) transducing the T cell with an expression vector comprising a nucleic acid sequence encoding a CAR.

In another aspect, the disclosure provides a method for treating a tumor in a subject, the method comprising: administering a genetically modified T cell of the disclosure to the subject, wherein the modified T cell kills tumor cells in the subject, thereby treating the tumor.

In another aspect, the disclosure provides a method for increasing the anti-tumor activity of a T cell, the method comprising contacting a tumor cell with a genetically modified T cell of the disclosure, wherein contacting the tumor cell with the genetically modified T cell increases the expression of anti-tumor cytokines or kills the tumor cell, thereby increasing the anti-tumor activity compared to a control T cell. In some embodiments, the control T cell does not comprise an expression vector of (i) or (ii) or both (i) and (ii) above.

In some embodiments, the method is an in vitro method. In some embodiments, the method is an in vivo method.

In another aspect, the disclosure provides a guide array comprising a plurality of CRISPR-associated RNA (crRNA) molecules, wherein the crRNA molecules comprise a direct repeat sequence and a spacer sequence that binds a target RNA, and wherein the crRNA molecules bind to different target mRNAs or different regions of the same target mRNA.

In any of the embodiments of the disclosure, the class 2 type VI-D CRISPR effector having RNA-guided RNA endonuclease activity is a Cas13 nuclease selected from the group consisting of Cas13a, Cas13b, 13bt1, Cas13bt2, Cas13c, Cas13d, RfxCas13d, Cas13e, and functional variants thereof.

In any of the embodiments of the disclosure, the guide array is a multicistronic array comprising a plurality of crRNA molecules.

In any of the embodiments of the disclosure, the guide array comprises from 2 to greater than or equal to 10 crRNA molecules.

In any of the embodiments of the disclosure, the guide array comprises crRNA molecules that bind to mRNA encoding a protein selected from the group consisting of TOX, TOX2, NR4A1, NR4A2, NR4A3, TET2, IRF4, JUNB, BATF3, DHX37, FLI1, ZC3H12A, SOCS1, TCEB2, PDCD1 (PD-1), HAVCR2 (TIM3), LAG3, CTLA4, TIGIT, FAS, TRAC, CBLB. RASA2, PTPN2, CD5, ENTPD1, CD46, B2M, and combinations thereof.

In any of the embodiment of the disclosure, the T cell or the primary T cell further comprises a chimeric antigen receptor (CAR).

In any of the embodiments of the disclosure, the CAR binds to an antigen expressed by a tumor.

In any of the embodiments of the disclosure, the CAR binds to an antigen selected from the group consisting of Her-2, B7-H3, GPC2, GD2, CD19, CD20, CD22, MAGE, BAGE, CAGE, GAGE, HAGE, LAGE, PAGE, PRAME, NY-ESO-1, NY-SEO-1, tyrosinase, Melan-A/MART, gpl00, TRP-1, TRP-2, CD30, EGFR, EGFRvIII, FAP, CD33, CD123, PD-L1, IGF1R, CD4, CSPG4, B7-H4, NKG2D, CS1, CD138, EpCAM, EBNA3C, GPA7, CD244, CA-125, ETA, CEA, CD52, MUCSAC, c-Met, FAB, WT-1, PSMA, AFP, BCMA, Mesothelin, GPC3, MUC1 and CTAG1B.

In any of the embodiments of the disclosure, expression and intracellular signaling by the CAR upregulates T cell exhaustion markers in control T cells compared to T cells comprising: (i) an expression cassette comprising a nucleic acid sequence encoding a class 2 type VI-D CRISPR effector having RNA-guided RNA endonuclease activity; and (ii) an expression cassette comprising a nucleic acid sequence encoding a guide array comprising a plurality of crRNA molecules, wherein the crRNA molecules comprise a direct repeat sequence and a spacer sequence that binds a target mRNA, and wherein the crRNA molecules bind to different target mRNAs or different regions of the same target mRNA.

In any of the embodiments of the disclosure, the exhaustion markers are selected from LAG3, PDCD1 (PD-1), or HAVCR2 (TIM3), or a combination thereof.

In any of the embodiments of the disclosure, the guide array comprises crRNA molecules that bind to mRNA encoding LAG3, PDCD1 (PD-1), or HAVCR2 (TIM3), or a combination thereof.

In any of the embodiments of the disclosure, the guide array comprises crRNA molecules that bind to mRNA expressed by the CAR.

In any of the embodiments of the disclosure, the T cell is selected from the group consisting of a human T cell and a primary human T cell.

### BRIEF DESCRIPTION OF THE DRAWINGS

FIG. 1. MEGA HA-282 CAR T cells robustly suppress exhaustion-related inhibitory receptor upregulation. (A) Schematic of relevant lentiviral constructs in MEGA CAR T cells. CRISPR/Cas13d enables multiplexed RNA targeting from a single guide array for efficient suppression of inhibitory receptor upregulation. DR: direct repeat. (B) Overview of optimized workflow to generate MEGA CAR T cells from primary human T cells for phenotypic evaluation. FACS: fluorescence-activated cell sorting. (C) Violin and box plot overlays depicting surface LAG3, PD-1, and TIM3 expression for single and double guides as measured by flow cytometry on day 10 from 1 representative donor. On-target guides are colored (LAG3, yellow; PD-1, red; TIM3, blue); off-target guides are grey. Dashed lines indicate median fluorescence intensity (MFI) values of either mock untransduced cells or exhausted non-targeting cells used for normalization. Labelled numbers indicate normalized values for each condition (mock MFI=0; non-targeting MFI=1). NT: non-targeting guide. (D) Aggregate normalized surface LAG3 (L), PD-1 (P), and TIM3 (T) expression as described in (C) for single and double guides across n=2-3 donors from independent experiments. Dashed line represents non-targeting MFI. *p<0.05, **p<0.005, ***p<0.0005, ****p<0.0001, ordinary one-way ANOVA with Dunnett's multiple comparisons test. Error bars are #s.d. (E) Correlation plots between mRNA transcript expression relative to NT (as measured by RT-qPCR) and normalized surface LAG3, PD-1, and TIM3 expression for single and double guides from 1 representative donor. On-target guides are pink; off target guides are black Colored lines represent best fit from linear regression. Error bars are s.e.m. (F) Volcano plots representing bulk RNA-Seq data from day 10 MEGA CAR T cells, n=2 donors from independent experiments. Significantly differentially-expressed genes in comparison to NT are highlighted (log 2fc <−1, padj <0.001) (G) Aggregate normalized surface LAG3, PD-1, and TIM3 expression for triple guide arrays across n=2-3 donors from independent experiments. Dashed line represents non-targeting MFI. ***p<0.0005, ****p<0.0001, ordinary one-way ANOVA with Dunnett's multiple comparisons test. Error bars are ±s.d. (H) Correlation plots between mRNA transcript expression relative to NT and normalized surface LAG3, PD-1, and TIM3 expression for triple guide arrays from 1 representative donor. Colored lines represent best fit from linear regression. Error bars are s.e.m. (I) Pie charts depicting relative percentages of LAG3+/−PD-1+/− TIM3+/− MEGA CAR T cells for triple guide arrays as measured by flow cytometry on day 10 from 1 representative donor. Triple positive cells are highlighted in yellow; triple negative cells are highlighted in blue.

FIG. 2. Combinatorial CRISPR screen in dysfunctional CAR T cells identifies paired regulators of proliferation. (A) Overview of 2D CRISPR screening strategy in dysfunctional CD8⁺ HA-28z CAR T cells. (B) Volcano plot depicting differential abundance (log 2 fold-change, 12fc) of guide arrays between early (plasmid DNA) and late (day 13) timepoints for n=2 replicates. Blue dots: significantly depleted arrays (adjusted p<0.05, 12fc <−0.5); red dots: significantly enriched arrays (adjusted p<0.05, 12fc>0.5); open black dots: non-targeting arrays. Adjusted p values from Wald test with Benjamini-Hochberg correction as implemented in DESeq2. (C) 2D heatmap of guide array enrichment between early and late timepoints. Upper-left: targeting double arrays; upper right and lower-left: targeting single arrays; lower-right: non-targeting arrays. (D) Ranked guide arrays using likelihood-ratio test as implemented in DESeq2 with n=2 replicates. Top-ranking arrays with most significant count variation across all screening timepoints are colored (blue: depleted; red: enriched). (E) Top: Histogram depicting the distribution of 12fcs between early and late timepoints for all 6,400 guide arrays in the library. Bottom: Rug plots depicting 12fcs for all 9 guide arrays targeting either significantly enriched (red) or depleted (blue) gene pairs, over the 12fc distribution of all 64 non-targeting guides (grey). (F) Fold-change expansion of FACS-sorted RfxCas13d+CD8⁺ HA-28z CAR T cells compared to a non-targeting control over 15 days of culture from n=3-4 donors from independent experiments. Significantly enriched guide arrays in red; significantly depleted guide arrays in blue; non-targeting control guide in black. Dashed line represents expansion of nontargeting control. p<0.0001, ordinary one-way ANOVA. Error bars are ±s.e.m.

FIG. 3. Paired transcriptomic perturbations broadly enhance the anti-tumor activity of dysfunctional MEGA CAR T cells. (A) Overview of experimental workflow to generate dysfunctional MEGA CAR T cells expressing validation arrays. (B) Schematic detailing experimental conditions for cytokine secretion assays. (C, D) Secretion of IFNγ (C) or IL-2 (D) after 24 hours of culture with (pink dots, stimulated) or without (black dots, baseline) antigen-positive tumor cells at 1:1 E: T. Data are mean values of n=3 replicate wells from 1 representative donor. *p<0.05, **p<0.005, ***p<0.0005. ****p<0.0001, ordinary one-way ANOVA with Dunnett's multiple comparisons test. Error bars are ±s.d. (E) Top: schematic detailing serial stimulation assay. MEGA CAR T cells are repeat challenged with antigen-positive tumor cells every 48-72 hours at 1:1 E: T. Bottom: Kinetics of tumor killing (1:1 E: T) as measured using Incucyte live-cell imaging. Data are mean values of n=3 replicate wells from 1 representative donor. Red traces: enriched guide arrays; black traces: non-targeting control. p<0.0001, repeated measures one-way ANOVA. Shaded regions are ±s.e.m. (F) Incucyte live-cell imaging as in FIG. 5E at a lower 1:5 E: T. (G) Incucyte images from 0 hr and 48 hr timepoints after three rounds of tumor stimulation as in FIG. 3E. Green: Nalm6-GD2 tumor cells; red: MEGA HA-28z CAR T cells. (H) Schematics for hypothesized mechanism of enhanced anti-tumor activity in dual CBLB+ FAS knockdown CAR T cells (bottom) compared to a non-targeting control (top).

FIG. 4. MEGA enables rapid, quantitative, and reversible control of the T cell transcriptome. (A) Schematic detailing exogenous regulation of RfxCas13d-DD expression by the small-molecule drug trimethoprim (TMP). (B) Violin and box plot overlays depicting surface CD46 expression as measured by flow cytometry on day 5 from 1 representative donor. On-target conditions are shown in purple; off-target conditions are shown in grey. Numbers above plots indicate pairwise fold-changes in CD46 expression. (C) Left: violin and box plot overlays depicting TMP-dependent surface CD46 expression as measured by flow cytometry on day 5 from 1 representative donor Right: dose-response curve depicting TMP-dependent titration of CD46 knockdown as measured by flow cytometry on day 5. Data are mean of n=3 replicate wells from 1 representative donor. Linear regime of sigmoidal curve shown in pink Error bars are ±s.d. (D) Time-course depicting kinetics of CD46 knockdown after TMP addition (left, yellow) or removal (right, dark blue) as measured by flow cytometry across 72 hours. Data are mean of n=3 replicate wells from 1 representative donor. Error bars are ±s.d. (E) Violin and box plot overlays depicting surface LAG3, PD-1, and TIM3 expression with or without TMP as measured by flow cytometry on day 10 from 1 representative donor. Colored histograms: on-target conditions; grey histograms: off-target conditions. Dashed lines indicate MFI values of either mock untransduced cells or exhausted non-targeting cells used for normalization. Labelled numbers indicate normalized values for each condition. (F) Violin and box plot overlays depicting surface LAG3, PD-1, and TIM3 expression over increasing concentrations of TMP as measured by flow cytometry on day 10 from 1 representative donor. (G) Dose-response curves depicting TMP-dependent titration of LAG3 (yellow), PD-1 (red), or TIM3 (blue) knockdown as measured by flow cytometry on day 10. Data are mean of n=3 replicate wells from 1 representative donor. Error bars are ±s.d.

FIG. 5. MEGA enables massively-multiplexed knockdown of immune-relevant endogenous genes in primary human T cells. (A) Overview of optimized two-step method for rapid and facile assembly of arbitrarily long guide arrays via overlap extension PCR. (B) mRNA transcript levels of LAG3, FAS, CTLA4, PD-1, and TIM3 relative to NT as measured by RT-qPCR on day 10 from n=2 donors. Numbers labelled within bars represent mean values. ****p <0.0001, Welch's t-test. Error bars are ±s.d. (C) Violin and box plot overlays depicting surface LAG3, FAS, CTLA4, PD-1, and TIM3 expression as measured by flow cytometry on day 10 from 1 representative donor. On-target guide array is coloured (LAG3, yellow;

FAS, green; CTLA4, dark blue; PD-1, red; TIM3, light blue); controls are grey. Dashed lines indicate MFI values of either mock or non-targeting cells. (D) Aggregate surface LAG3, FAS, CTLA4, PD-1, and TIM3 expression relative to NT across n=3 donors from independent experiments. Dashed lines represent MFI of either mock or non-targeting cells. **p<0.005, ***p<0.0005, ordinary one-way ANOVA with Dunnett's multiple comparisons test. Error bars are ±s.d. (E) mRNA transcript levels of LAG3, FAS, CD5, CD39, CD46, TRAC, B2M, CTLA4, PD-1, and TIM3 relative to NT as measured by RT-qPCR on day 10 from n=2 donors. Numbers labelled within bars represent mean values. *p<0.05, **p<0.005, ***p <0.0005, ****p<0.0001, Welch's t-test. Error bars are ±s.d.

FIG. 6. Metabolic engineering boosts CAR T cell function via whole-pathway disruption of the purinergic signaling cascade. (A) Schematic detailing the pathways involved in purinergic signaling and adenosine-mediated immunosuppression. (B) Schematic detailing the anticipated effects of purinergic signaling disruption on metabolite levels and immune activity. (C) mRNA transcript levels of purinergic signaling components A2BR, A2AR, CD73, and CD39 relative to NT as measured by RT-qPCR on day 10, n=3 technical replicates from 1 representative donor. Numbers labeled within bars represent mean values. ****p<0.0001, Welch's t-test. Error bars are ±s.d. (D, E, F) Concentrations of metabolites ATP (D), AMP (E), and adenosine (F) in the culture medium of MEGA HA-28z CAR T cells expressing the PURI array (green) or a non-targeting guide (grey). Data are mean values of n=2-3 replicate wells from 1 representative donor. *p<0.05, **p<0.005, unpaired t-test. Error bars are ±s.d. (G, H) Time-course depicting the concentrations of metabolites ATP (G) and AMP (H) in the culture medium of MEGA HA-28z CAR T cells after spike-in with 20 μM ATP. Green traces: PURI array; grey traces: non-targeting control. Data are mean values of n=3 replicate wells from 1 representative donor. **p<0.005, ****p<0.0001, repeated measures two-way ANOVA, column-factor. Error bars are ±s.d. (I, J) Secretion of IFNγ (I) or IL-2 (J) by dysfunctional MEGA CAR T cells after 24 hours of culture with (stimulated) or without (baseline) antigen-positive tumor cells at 1:1 E: T. Data are mean values of n=3 replicate wells from 1 representative donor Green bars: PURI array; grey bars. NT control. ****p<0.0001, two-way ANOVA with Bonferroni's multiple comparisons test. Error bars are ±s.d. (K, L) Kinetics of tumor killing (K) and T cell proliferation (L) as measured using Incucyte live-cell imaging at a 1:1: E: T ratio. Data are mean values of n=3 replicate wells from 1 representative donor. Green trace: PURI array; grey trace: NT control. **p<0.005, repeated measures two-way ANOVA, column-factor. Shaded regions are+s.e.m.

FIG. 7. MEGA is a flexible and multi-functional platform for transcriptomic regulation in primary human T cells using CRISPR/Cas13d.

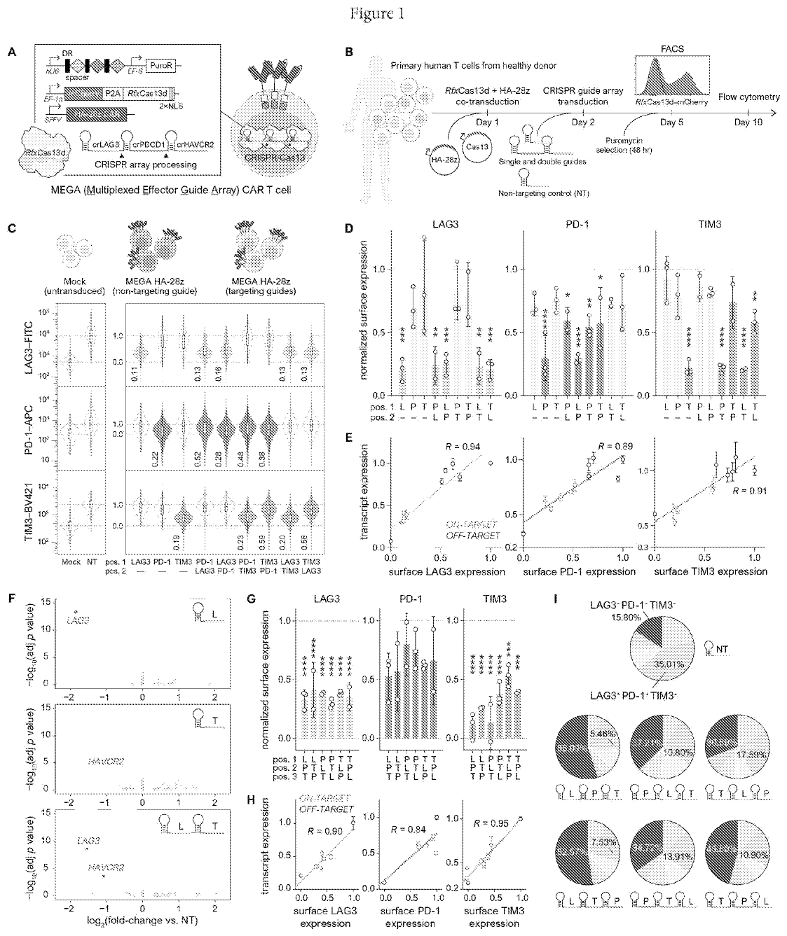

FIG. 8, related to FIG. 1. Characterization and optimization of RfxCas13d expression and activity in primary human T cells. (A) Overview of lentiviral constructs used and experimental setup for GFP reporter repression experiment. (B) GFP expression as measured by flow cytometry on day S with increasing amounts of GFP virus. Green line: cells transduced with RfxCas13d: GFP and GFP reporter; dotted black line: cells transduced with GFP reporter only. Data are mean of n=3 replicate wells from 1 representative donor. (C) Overview of lentiviral constructs used for repression of exhaustion markers in HA-28z CAR T cells. (D) Expression of LAG3, PD-1, and TIM3 relative to NT as measured by flow cytometry on day 10 across n=3 donors from independent experiments. Colored bars: on-target guides (LAG3, yellow; PD-1, red; TIM3, blue); grey bars: off-target guides. (E) Histogram depicting expression of RfxCas13d as measured by flow cytometry on day 10 from 1 representative donor. Black unfilled histogram: mock untransduced cells; magenta filled histogram: cells transduced with RfxCas13d. (F) Violin and box plot overlays depicting RfxCas 13d expression as measured by flow cytometry on day 5 from 1 representative donor. Black unfilled histogram: mock untransduced cells; magenta filled histogram: cells transduced with RfxCas13d constructs in various configurations. (G) Model describing the context-dependent effects of RfxCas13d activity in LX293T cells on lentiviral titer during viral packaging. (H, I) Violin plots depicting (H) RfxCas13d and (I) HA-28z CAR expression of co-transduced primary human T cells as measured by flow cytometry on day 5 with increasing amounts of virus.

FIG. 9, related to FIG. 1. Multiplexed repression of exhaustion markers in MEGA HA-28z CAR T cells. (A) mRNA transcript expression of LAG3, PD-1, and TIM3 for single and double guides as measured by RT-qPCR on day 10 (see FIG. 1E). Data are mean of n=3 technical replicates from 1 representative donor. Error bars are ±s.d. M: mock; N: non-targeting; L: LAG3; P. PD-1; T: TIM3. (B) Violin and box plot overlays depicting HA-28z CAR expression for single and double guides on day 5 as measured by flow cytometry from 1 representative donor. Blue histograms: transduced cells; grey histogram: mock untransduced cells. (C) Violin and box plot overlays depicting surface LAG3, PD-1, and TIM3 expression for triple guides as measured by flow cytometry on day 10 from 1 representative donor (see FIG. 1F). On-target guides are colored (LAG3, yellow; PD-1, red; TIM3, blue);

controls are grey. Dashed lines indicate MFI values of either mock untransduced cells or exhausted nontargeting cells used for normalization. Labelled numbers indicate normalized values for each condition. (D) mRNA transcript expression of LAG3, PD-1, and TIM3 for triple guides as measured by RT-qPCR on day 10 (see FIG. 1H). Data are mean of n=3 technical replicates from 1 representative donor. Error bars are ±s.d. (E) Violin and box plot overlays depicting HA-28z CAR expression for triple guides on day 5 as measured by flow cytometry from 1 representative donor. Blue histograms: transduced cells; grey histogram: mock untransduced cells. (F, G) Gating strategy used in FIG. 1I to determine LAG3+/−PD-1+/− TIM3+/− populations in unsorted MEGA HA-28z CAR T cells expressing (F) non-targeting control or (G) triple guide array. Data are measured by flow cytometry on day 10 from 1 representative donor. From left to right: live cell (lymphocyte) gate, singlet gate, RfxCas13d+ gate, LAG3+/−PD-1+/− gate, TIM3+/− gate.

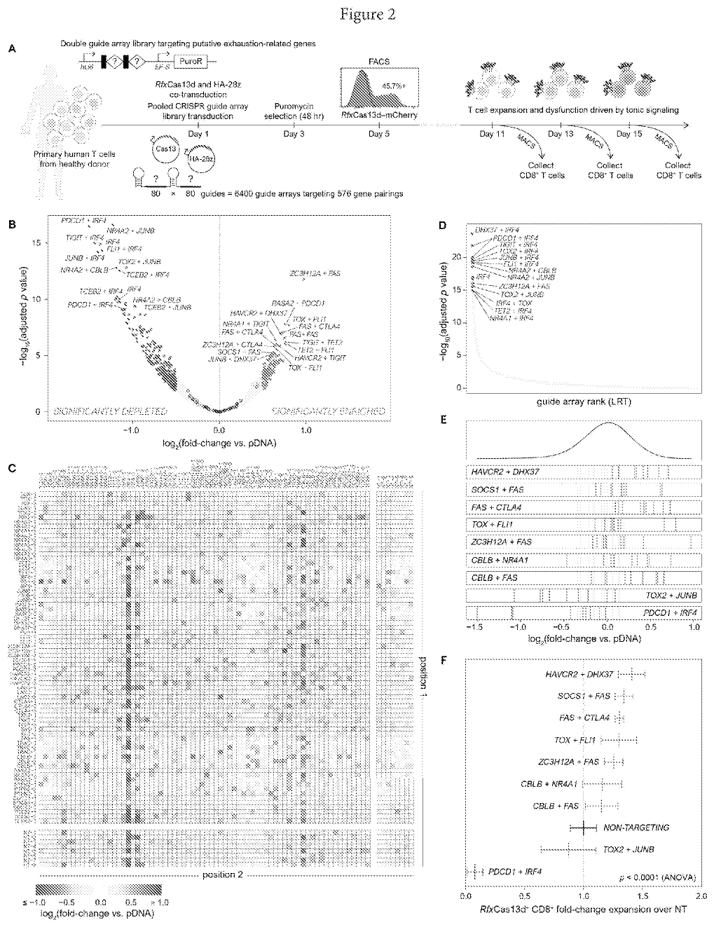

FIG. 10, related to FIG. 2. Further characterization of Cas13-based CRISPR screen in HA-28z CAR T cells. (A) Plot representing the distribution of guide arrays in the plasmid DNA prep of the assembled custom library as determined by NGS. Guide array counts are ranked in order of abundance. Dashed lines represent 90th and 10th percentiles. Blue labelled numbers indicate common metrics to quantify library coverage and bias. (B) Histograms depicting RfxCas13d (left) and HA-28z CAR (right) expression over the duration of the screen. Colored histograms: cells for screening (RfxCas13d: magenta; CAR: blue); unfilled histograms: mock untransduced cells. (C) Plots representing the distribution of guide arrays over the duration of the screen across two replicates. (D) Plots depicting guide array abundance correlation between two replicates. Left: early timepoint; right: late timepoint. (E) Volcano plot depicting the average differential abundance of gene pairs between early (plasmid DNA) and late (day 13) timepoints for n=2 replicates. Blue dots: significantly depleted pairs; red dots: significantly enriched pairs; open black dot: non-targeting control. p values from robust rank aggregation as implemented in MAGeCK. (F) 2D heatmap of gene pair enrichment between early and late timepoints. Genes are ordered by hierarchical clustering as implemented in pheatmap.

FIG. 11, related to FIG. 2. Validation and phenotyping of 2D CRISPR screen hits. (A) Violin and box plot overlays depicting HA-28z CAR expression (top) and RfxCas13d expression (bottom) for validation arrays on day 5 (before FACS) as measured by flow cytometry from 1 representative donor. Dashed line represents gate for RfxCas13d+ cells. Red histograms: enriched arrays; blue histograms: depleted arrays; grey histograms: controls. (B) Aggregate % RfxCas13d+as detailed in (A) across n=3 donors from independent experiments. Red bars: enriched arrays; blue bars: depleted arrays; grey bar: non-targeting. **p<0.005, ****p<0.0001, ordinary one-way ANOVA with Dunnett's multiple comparisons test. Error bars are ±s.d. (C) Proliferation traces for FACS-sorted RfxCas13d+CD8⁺ HA-28z CAR T cells over 15 days in culture (see FIG. 2F). Each panel corresponds to a new donor from an independent experiment. Red lines: enriched arrays; blue lines: depleted arrays; black line: non-targeting; shaded grey region: AUC for non-targeting control. (D) Fold-change expansion of FACS-sorted RfxCas13d+HA-28z CAR T cells compared to a non-targeting control over 15 days of culture from n=3-4 donors from independent experiments. Significantly enriched guide arrays in red; significantly depleted guide arrays in blue; non-targeting control guide in black. Dashed line represents expansion of non-targeting control. p<0.0001, ordinary one-way ANOVA. Error bars are±s.e.m. (E) Proliferation traces for FACS-sorted RfxCas13d+HA-28z CAR T cells over 15 days in culture across n=4 donors from independent experiments (one panel each). Red lines: enriched arrays; blue lines: depleted arrays; black line: non-targeting; shaded grey region: AUC for non-targeting control. (F, G) Density plots depicting levels of CD62L and CD45RA on HA-28z CAR T cells expressing non-targeting (F) or targeting (G) guide arrays as measured by day 10 flow cytometry from 1 representative donor. Labelled numbers in each quadrant denote the relative frequency of the indicated T cell subset (pink: greatest global frequency).

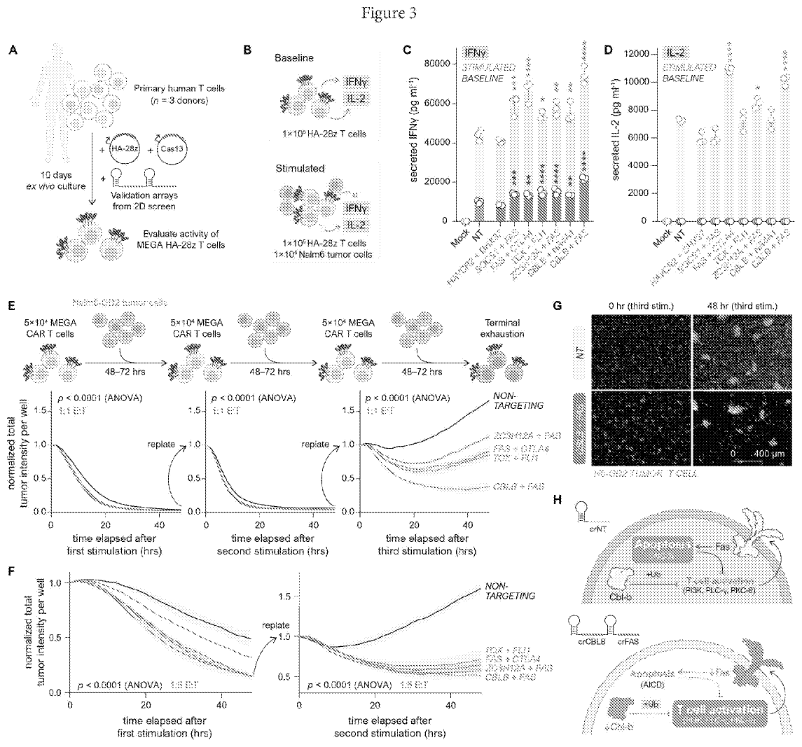

FIG. 12, related to FIG. 3. Evaluation of dysfunctional MEGA CAR T cell anti-tumor activity. (A, B) Secretion of IFNγ (A) or IL-2 (B) by dysfunctional MEGA CAR T cells after 24 hours of culture with (pink dots, stimulated) or without (black dots, baseline) antigen-positive tumor cells at 1:1 E: T, as in FIGS. 3B-C. Data are mean values of n=3 replicate wells. Each panel corresponds to a new donor from an independent experiment. *p<0.05, **p <0.005, ***p<0.0005, ****p<0.0001, ordinary one-way ANOVA with Dunnett's multiple comparisons test Error bars are ±s.d. (C) Kinetics of tumor killing (1:1 E: T) as measured using Incucyte live-cell imaging, as in FIG. 3D. Data are mean values of n=3 replicate wells. Each row corresponds to a new donor from an independent experiment. Red traces: enriched guide arrays; blue traces: depleted guide arrays; black traces: non-targeting control. p<0.0001, repeated measures one-way ANOVA with Dunnett's multiple comparisons test. Shaded regions are±s.e.m.

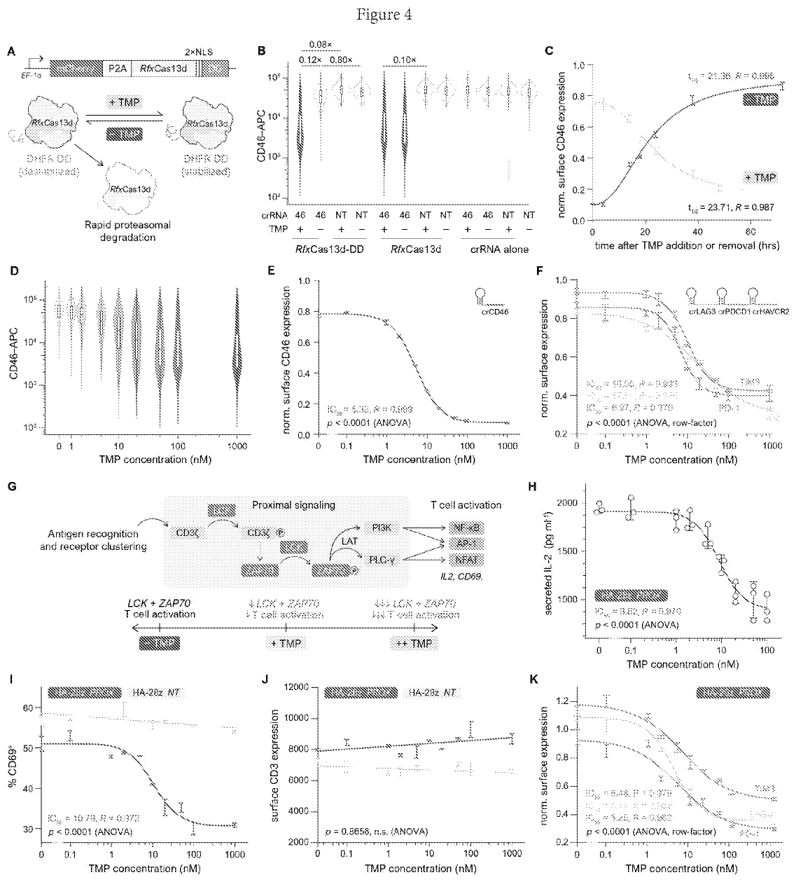

FIG. 13, related to FIG. 4. Regulation of RfxCas13d activity by a small-molecule drug. (A) Density plots representing expression of RfxCas13d and LAG3/PD-1/TIM3 in day 10 MEGA HA-28z CAR T cells as measured by flow cytometry from 1 representative donor. Left column: targeting guides; right column: non-targeting control. (B) Violin and box plot overlays depicting intracellular RfxCas13d expression on day 5 as measured by FLAG tag staining and flow cytometry from 1 representative donor. Blue histograms: high expression conditions; grey histogram: low expression conditions. (C) Violin and box plot overlays depicting surface CD46 as measured by flow cytometry across 72 hours from 1 representative donor (see FIG. 4D). Top: TMP addition; bottom: TMP removal. Solid filled histograms: TMP addition (yellow) or removal (dark blue); dotted unfilled histograms: non-targeting control. (D) Time-course depicting kinetics of CD46 degradation after CHX addition as measured by flow cytometry. Data are mean of n=3 replicate wells from 1 representative donor. Error bars are ±s.d. (E) Bar plots depicting CD46 expression (left), mCherry expression (center), or cell viability (right) with varying concentrations of TMP. Colored bars: CD46 targeting guide (CD46 expression: purple; mCherry expression: magenta, cell viability: blue); grey bars: non-targeting guide. Numbers labelled above bars indicate fold-change over NT: Data are mean of n=3 replicate wells from 1 representative donor. *p<0.05. **p<0.005, ****p<0.0001, multiple unpaired t tests with FDR correction (Benjamini, Krieger, and Yekutieli). Error bars are ±s.d.

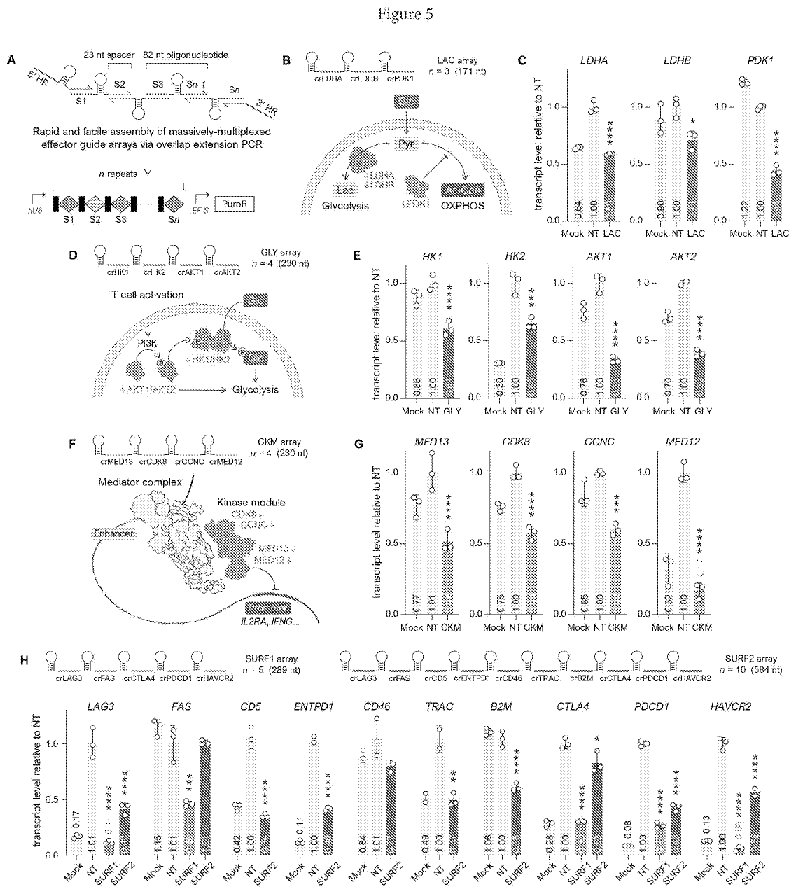

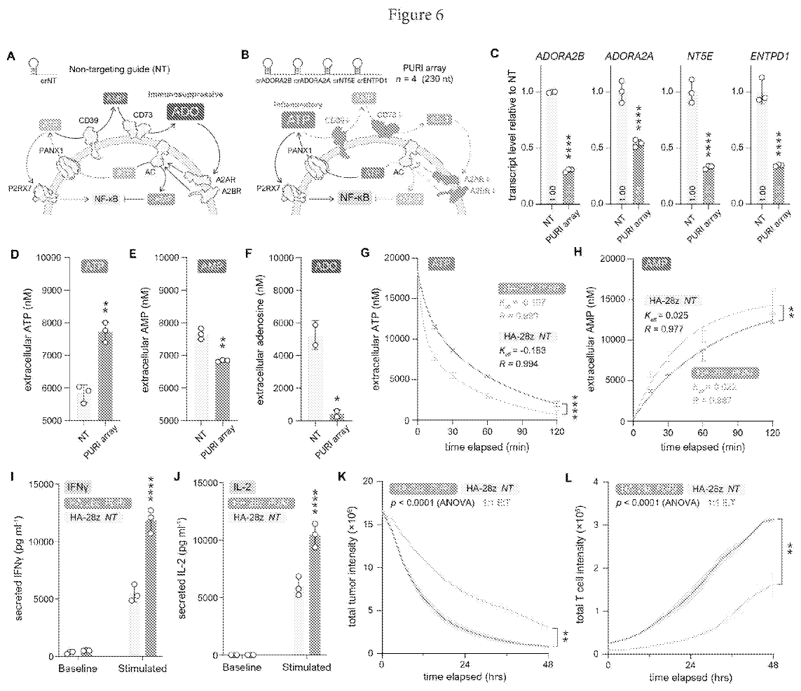

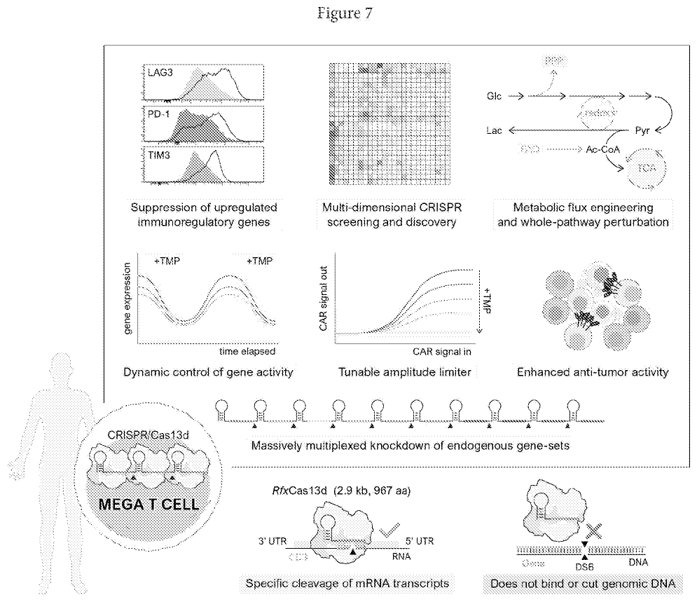

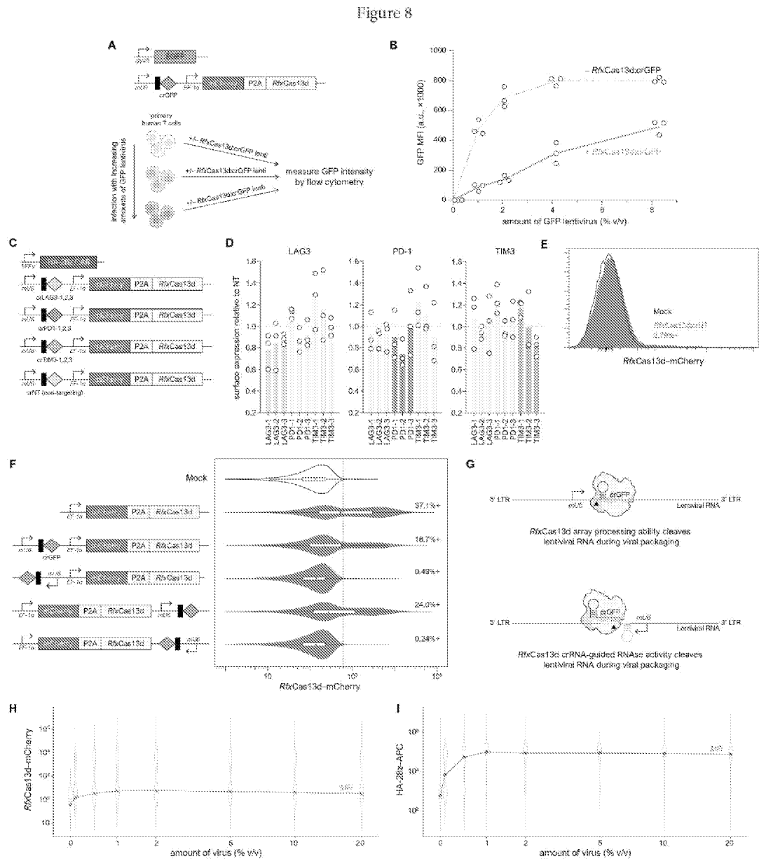

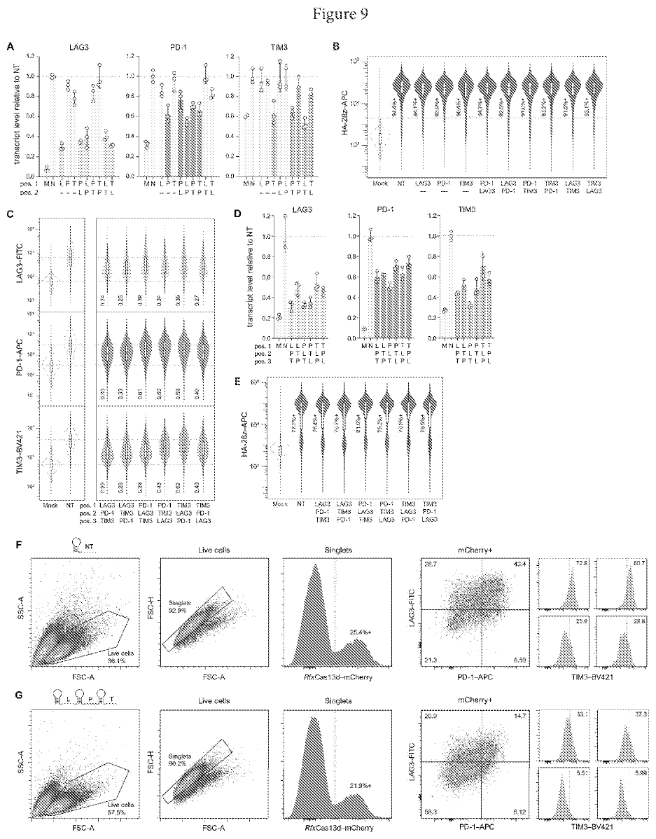

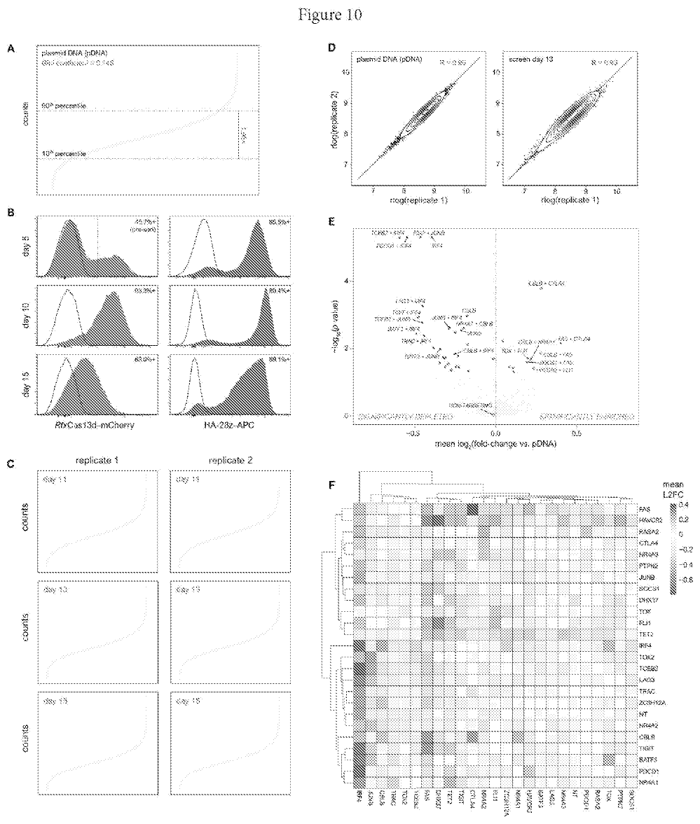

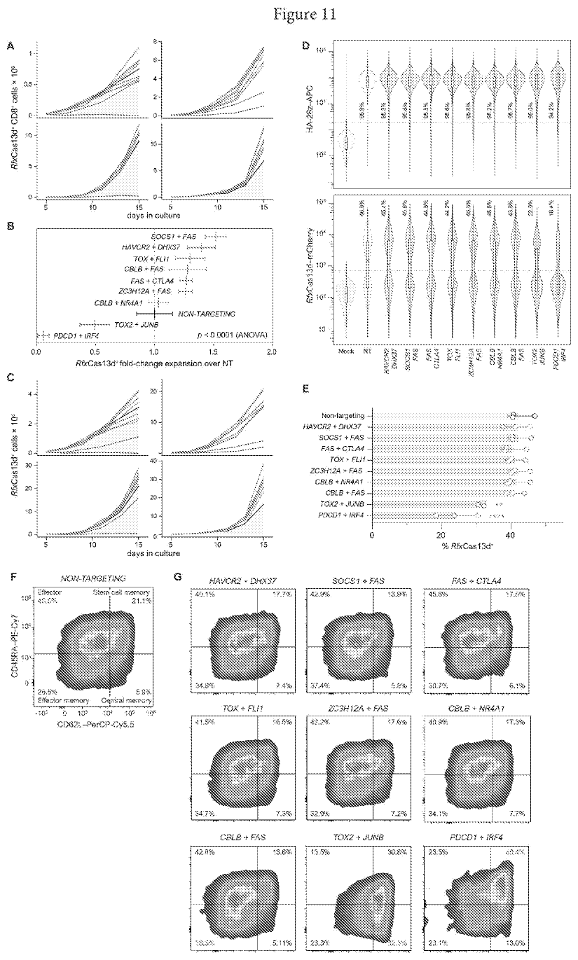

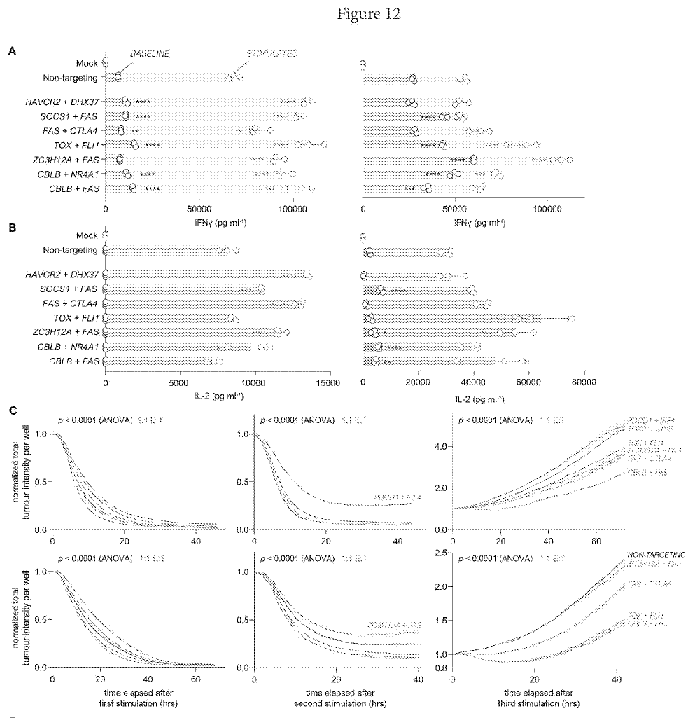

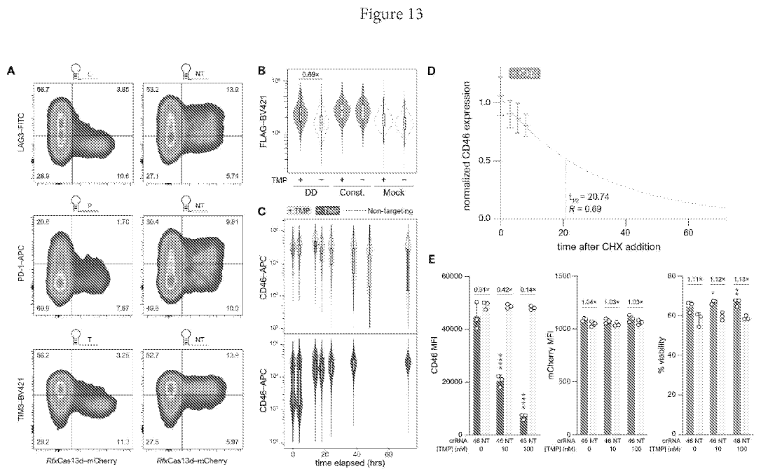

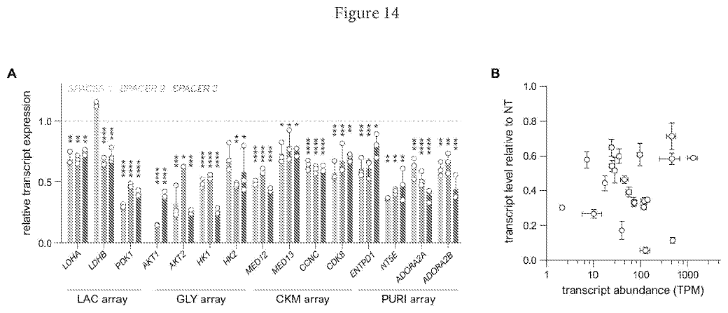

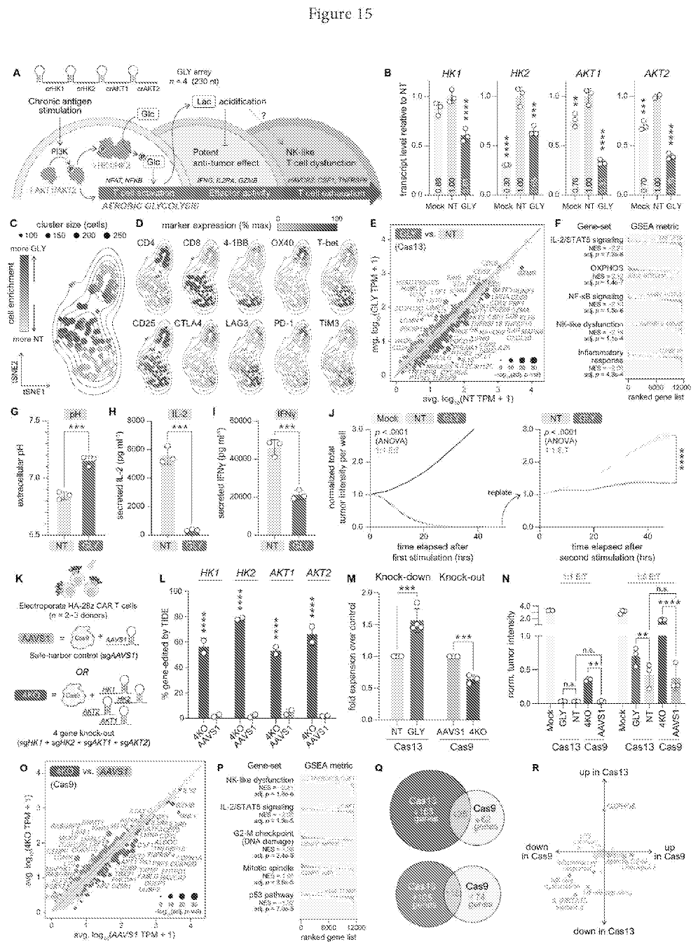

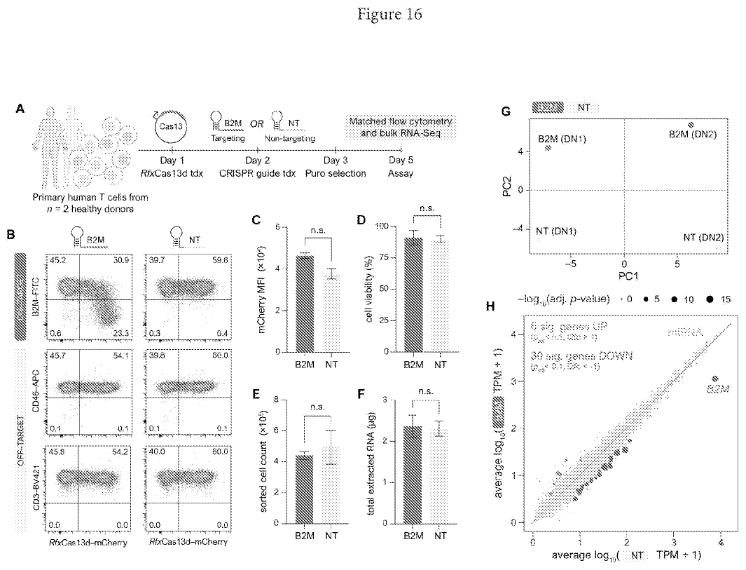

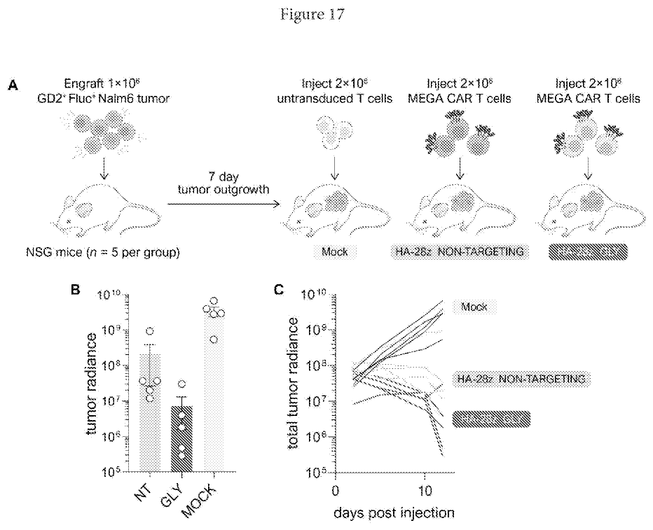

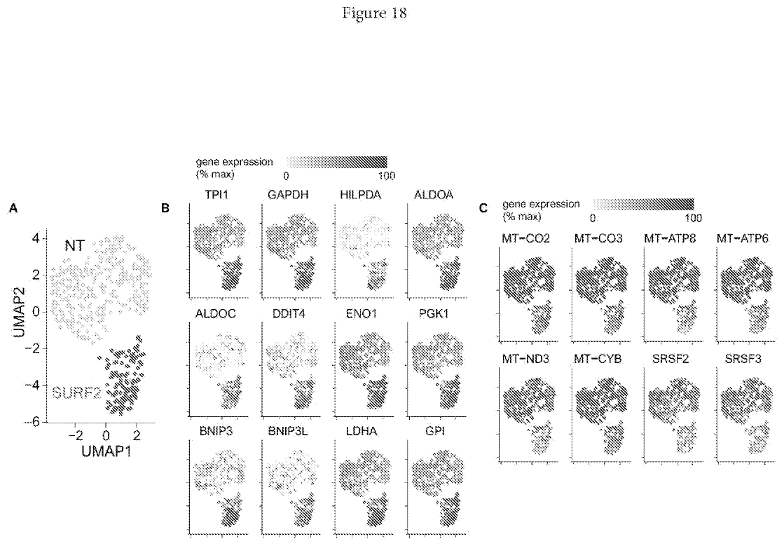

FIG. 14, related to FIG. 5. Gene knockdown is robust across spacers and target transcript abundances. (A) mRNA transcript levels of all targeted genes in FIG. 5 (three spacers per transcript) relative to NT as measured by RTqPCR on day 10, n=3 technical replicates from one donor. *p<0.05. **p<0.005, ***p<0.0005, ****p<0.0001, ordinary one-way ANOVA with Dunnett's multiple comparisons test. Error bars are ±s.d. (B) Relative transcript expression plotted against transcript abundance as measured by bulk RNA-Seq of HA-28z CAR T cells for each gene targeted in FIG. 5.

FIG. 15. Disruption of PI3K/Akt-driven glycolytic activity modulates CAR T cell metabolism and mitigates exhaustion. (A) Schematic depicting aerobic glycolysis as a metabolic driver of T cell effector differentiation and eventual exhaustion. The four-gene guide array (GLY) perturbs the upstream glycolytic genes HK1, HK2, AKT1, and AKT2. (B) mRNA transcript levels of HK1, HK2, AKT1, and AKT2 relative to NT as measured by RT-qPCR on day 10, n=3 technical replicates from one donor representative of two independent experiments. Numbers labeled within bars represent mean values. **p<0.01, ***p<0.001, ****p<0.0001, ordinary one-way ANOVA with Dunnett's multiple comparisons test. Error bars are ±s.d. (C) Two-dimensional t-distributed stochastic neighbor embedding (t-SNE) visualization of clustered high-dimensional mass cytometry (CyTOF) data from day 10 MEGA HA-28z CAR T cells expressing either the GLY array (11=9871 cells) or a non-targeting (NT) guide (n=8025 cells). Dots represent clusters of cells with similar protein expression profiles as determined by unsupervised FlowSOM clustering. Dot positions represent cluster centroids. Dot sizes represent the number of cells represented by a certain cluster. Dot coloring represents the enrichment (log 2 fold-change count) of cells in each cluster labeled GLY (dark green) or NT (yellow) after de-barcoding. Contour lines represent the kernel density estimation in the two-dimensional latent space. (D) t-SNE visualization of cell clusters as described in (C) colored according to normalized marker expression (% max). High-expressing cell clusters are colored dark purple, while low-expressing cell clusters are colored light orange. (E) Transcriptome-wide quantification of bulk RNA-seq data from day 10 MEGA HA-28z CAR T cells with the targeting GLY array (y-axis) or NT control (x-axis), averaged over n=2 donors from independent experiments. Each dot represents a single gene; gene-level expression was determined by aggregating transcripts per million (TPM) values and p-values across all detected transcripts for each gene. Differentially expressed genes with adjusted p-value <0.2 and absolute log 2 fold-change >0.5 are highlighted (red: upregulated in GLY; blue: downregulated in GLY). Dot sizes scale with statistical significance (−log 10 adjusted p-value). The green line indicates where gene expression is equal across both conditions (y=x). (F) Results from gene-set enrichment analysis (GSEA) including all Hallmark gene sets as well as the previously-described NK-like T cell dysfunction gene signature. The top 5 most significantly enriched gene sets are shown. Genes were ranked according to the following ranking metric=−log 10 (adj. p-value)*sign (log 2 fold-change GLY/NT). Labeled genes contributed most to the normalized enrichment score (NES). (G) Extracellular pH measurements of MEGA HA-28z CAR T cell culture supernatant after 48 hours in culture. Cells were plated at 1×106 cells per ml in triplicate wells. Data is representative of n=3 donors across independent experiments. ***p<0.001, unpaired t-test. Error bars are ±s.d. (H, I) Secretion of IL-2 (H) or IFNγ (I) by dysfunctional MEGA HA-28z CAR T cells after 24 hours of culture with antigen-positive tumor cells at 1:1 E: T. Data are mean values of n=3 replicate wells from 1 representative donor. Dark green bars: GLY array; yellow bars: NT control. ***p<0.001, unpaired t-test. Error bars are ±s.d. (J) Kinetics of tumor killing (1:1 E.T) in a repeat stimulation assay as measured using Incucyte live-cell imaging. Data are mean values of n=3 replicate wells from 1 representative donor. Black trace: mock T cells; yellow trace: non-targeting control; dark green trace. GLY array ****p<0.0001, repeated measures two-way ANOVA. Shaded regions are #s.e.m. (K) Schematic outlining the generation of Cas9 gene-edited HA-28z CAR T cells via RNP electroporation. HA-28z CAR T cells were electroporated on day 3 of culture with either Cas9 complexed with a control guide targeting the AAVS1 safe-harbor locus (AAVS1) or with a mixture of four guides targeting HK1, HK2. AKT1, and AKT2 (4KO). (L) Editing efficiency of four-gene knockout (4KO, dark blue bars) in comparison to control (AAVS1, cyan bars) as determined by TIDE analysis of PCR amplicons from the indicated genomic loci. n=2 donors across independent experiments. ****p<0.0001, two-way ANOVA with Bonferroni's multiple comparisons test. (M) Fold-change expansion of HA-28z CAR T cells with HK1, HK2, AKT1, and AKT2 knock-down (left) or knock-out (right) over 10 days in culture across n=3-4 donors from independent experiments. Left: GLY array (dark green) vs. NT control (yellow). Right: 4KO (dark blue) vs. AAVS1 control (cyan). ***p<0.001, unpaired t-test. Error bars are ±s.d. (N) Tumor killing by HA-28z CAR T cells at different effector: target ratios. Data represents endpoint (t=48 hr) tumor intensity normalized to initial (t=0 hr) tumor intensity. Grey: mock; dark green: Cas13 GLY array; yellow: Cas13 NT control; dark blue: Cas9 4KO; cyan: Cas9 AAVS1 control. **p <0.01, ****p<0.0001, two-way ANOVA with FDR-corrected multiple comparisons test (Benjamini, Krieger and Yekutieli). (O) Transcriptome-wide quantification of bulk RNA-seq data from day 10 HA-28z CAR T cells gene-edited with Cas9 4KO (y-axis) or Cas9 AAVS1 control (x-axis), averaged over n=2 donors from independent experiments. Each dot represents a single gene; gene-level expression was determined by aggregating transcripts per million (TPM) values and p-values across all detected transcripts for each gene. Differentially expressed genes with adjusted p-value <0.2 and absolute log 2 fold-change >0.5 are highlighted (red: upregulated in 4KO; blue: downregulated in 4KO). Dot sizes scale with statistical significance (−log 10 adjusted p-value). The green line indicates where gene expression is equal across both conditions (y=x). (P) Results from gene-set enrichment analysis (GSEA) including all Hallmark gene sets as well as the previously-described NK-like T cell dysfunction gene signature. The top 5 most significantly enriched gene sets are shown. Genes were ranked according to the following ranking metric=−log 10 (adj. p-value)*sign (log 2 fold-change 4KO/AAVS1). Labeled genes contributed most to the normalized enrichment score (NES). (Q) Area-proportional Euler diagrams depicting significant differentially expressed genes identified from (E) and (O). Top: downregulated genes; bottom: upregulated genes. The Cas13 gene set (n=188 down, n=123 up) represents genes that were significantly different between GLY and NT conditions; the Cas9 gene set (n=87 down, n=92 up) represents genes that were significantly different between 4KO and AAVS1 conditions. (R) GSEA results for Cas13 (GLY vs. NT) plotted against GSEA results for Cas9 (4KO vs. AAVS1). Dots represent the overall GSEA ranking metric=−log 10 (adj. p-value)*sign (NES) for each gene set. Gene sets included in (F) and (P) are labeled and colored by category: green dots are down in Cas13 and Cas9; blue dots are down in Cas9 only; magenta dots are up in Cas13 only; orange dots are up in Cas9 only.

FIG. 16. RfxCas13d does not exhibit collateral activity in primary human MEGA T cells. (A) Schematic depicting experimental workflow to evaluate RfxCas13d RNA-level and protein-level collateral activity resulting from on-target B2M cleavage in primary human T cells from n=2 donors. (B) Dot plots depicting protein expression levels of mCherry-P2A-RfxCas13d against either B2M (on-target), CD46 (off-target), or CD3 (off-target) for MEGA T cells expressing either a B2M-targeting guide (left column) or a non-targeting (NT) control (right column). Data are measured by flow cytometry on day 10 from 1 representative donor. Labeled numbers indicate the percentage of cells in each quadrant, respectively (C) Mean fluorescence intensity (MFI) of mCherry in MEGA T cells expressing either a B2M-targeting guide (purple bar) or a non-targeting control (grey bar). Data are mean values of n=2 donors. Error bars are ±s.d. p=0.0596 (n.s.), paired t-test. (D) Cell viability of MEGA T cells expressing either a B2M-targeting guide (purple bar) or a non-targeting control (grey bar). Data are mean values of n=2 donors. Error bars are ±s.d. p=0.8949 (n.s.), paired t-test. (E) Cell count (total yield) of FACS-sorted MEGA T cells expressing either a B2M-targeting guide (purple bar) or a non-targeting control (grey bar). Data are mean values of n=2 donors. Error bars are ±s.d. p=0.5410 (n.s.), paired t-test. (F) Total RNA extracted (total yield) from FACS-sorted MEGA T cells expressing either a B2M-targeting guide (purple bar) or a non-targeting control (grey bar). Data are mean values of n=2 donors. Error bars are: +s.d. p=0.8820 (n.s.), paired t-test. (G) Principal component analysis (PCA) of bulk RNA-seq data generated from FACS-sorted MEGA T cells expressing either a B2M-targeting guide (purple dots) or a non-targeting control (grey dots) from n=2 donors (DN). (H) Transcriptome-wide quantification of bulk RNA-seq data from day 5 MEGA T cells with the targeting B2M guide (y-axis) or NT control guide (x-axis), averaged over n=2 donors. Each dot represents a single gene, gene-level expression was determined by aggregating transcripts per million (TPM) values and p-values across all detected transcripts for each gene. Differentially expressed genes with adjusted p-value <0.1 and absolute log 2 fold-change >1 are highlighted (red: upregulated in B2M; blue. downregulated in B2M). The targeted B2M transcript is colored purple. Mitochondrial RNAs are colored orange. Dot sizes scale with statistical significance (−log 10 adjusted p-value). The green line indicates where gene expression is equal across both conditions (y=x).

FIG. 17. MEGA CAR T cells with disrupted glycolytic metabolism exhibit enhanced anti-tumor efficacy in vivo. (A) Schematic detailing experimental procedure for in vivo tumor challenge of MEGA HA-28z CAR T cells. On day 0, NSG mice were inoculated with 1e6 antigen-positive Nalm6-GD2 (GFP⁺ Luc+) One week later, mice were injected with 1) mock untransduced T cells, 2) MEGA HA-28z CAR T cells expressing a non-targeting control guide, or 3) MEGA HA-28z CAR T cells expressing the GLY (HK1/HK2/AKT1/AKT2) array. (B) Quantification of tumor burden in Nalm6-GD2 tumor-bearing mice through bioluminescence imaging (BLI) on day 12. grey bar: non-targeting control; dark-grey bar: GLY array; light grey bar: mock. Each dot represents a tumor BLI measurement per mouse. Data are mean values of n=5 mice per condition (error bars are ±s.e.m.). (C) Quantification of tumor burden in Nalm6-GD2 tumor-bearing mice through bioluminescence imaging (BLI) over 12 days. Dark-grey traces: MEGA T cells expressing GLY array; grey traces: MEGA HA-28z CAR T cells expressing a non-targeting control guide; light grey traces: mock untransduced T cells. n=5 mice per condition.

FIG. 18. Massively multiplexed knockdown of ten genes in MEGA HA-28z CAR T cells broadly and robustly reprograms the transcriptome at the single-cell level. (A) Two-dimensional Uniform Manifold Approximation and Projection (UMAP) visualization of single-cell RNA sequencing data (scRNA-seq) of MEGA HA-28z CAR T cells expressing either a ten-gene guide array (SURF2, blue dots, n=113 cells) or a non-targeting control (NT, grey dots, n=307 cells). (B) UMAP visualization of scRNA-seq data as in (A) colored according to normalized gene expression. Single cell expression data for the top 12 most significantly enriched genes in SURF2 cells as compared to NT are shown. (C) UMAP visualization of scRNA-seq data as in (A) colored according to normalized gene expression. Single cell expression data for the top 12 most significantly depleted genes in SURF2 cells as compared to NT are shown.

### DETAILED DESCRIPTION

### Introduction

The present disclosure provides compositions and methods for multiplexed transcriptomic regulation in cells, which provide a versatile and multi-functional platform for programmable and scalable regulation of the cellular transcriptome. The compositions and methods of the disclosure provide advantages over existing CRISPR gene editing technologies, which are limited in their safety, efficacy, and scope. The compositions and methods of the disclosure enable quantitative, reversible, and massively-multiplexed gene knockdown in cells without targeting or cutting genomic DNA.

The compositions and methods use class 2 CRISPR/Cas systems, which use a single RNA-guided protein effector with RNA-guided RNA endonuclease activity. In some embodiments, the class 2 CRISPR/Cas system comprises a type VI-D CRISPR effector. Unlike Cas9, type VI-D CRISPR effectors do not bind, target, or cut DNA⁴². Rather, type VI-D CRISPR effectors complex with a CRISPR-associated RNA (crRNA) containing a programmable spacer sequence that directs the ribonucleoprotein to specific RNA transcripts for targeted degradation.

Type VI-D CRISPR effector proteins are thought to be guided to their target RNAs by a single crRNA comprising a direct repeat stem loop sequence and a spacer sequence (guide RNA) that binds to the target sequence via RNA-RNA hybridization⁴⁹. The direct repeat sequence used depends on the particular class 2 type VI-D CRISPR effector protein that is co-expressed in the cell. For example, the direct repeats in guide RNAs that form complexes with Cas13d are highly conserved in length and secondary structure.

Representative, non-limiting examples of Type VI-D CRISPR effectors of the disclosure include the Cas 13 family, which includes Cas13a, Cas13b, 13bt1, Cas13bt2, Cas13c, Cas13d, RfxCas13d, Cas13e, and functional variants thereof. In some embodiments, the Type VI-D CRISPR effector is Cas13d. Cas13d has the unique ability to process poly-crRNA guide arrays into distinct crRNAs to facilitate efficient simultaneous targeting of multiple RNA transcripts in single cells^{42, 44}. Finally, Cas 13d is roughly two-thirds the size of wild-type Cas9 (and Cas9 fusion variants are even larger), making it highly amenable to T cell manufacturing

### General

The practice of the present disclosure employs, unless otherwise indicated, conventional techniques of immunology, biochemistry, chemistry, molecular biology, microbiology, cell biology, genomics and recombinant DNA, which are within the skill of the art. See Sambrook, Fritsch and Maniatis, Molecular Cloning: A Laboratory Manual, 2nd edition (1989), Current Protocols in Molecular Biology (F. M. Ausubel, et al. eds., (1987), the series Methods in Enzymology (Academic Press, Inc.): PCR 2: A Practical Approach (M. J. MacPherson, B. D. Hames and G. R. Taylor eds. (1995), Antibodies, A Laboratory Manual, and Animal Cell Culture (R. I. Freshney, ed. (1987).

Oligonucleotides that are not commercially available can be chemically synthesized, e.g., according to the solid phase phosphoramidite triester method first described by Beaucage and Caruthers, Tetrahedron Lett. 22:1859-1862 (1981), using an automated synthesizer, as described in Van Devanter et. al., Nucleic Acids Res. 12:6159-6168 (1984). Purification of oligonucleotides is performed using any art-recognized strategy, e.g., native acrylamide gel electrophoresis or anion-exchange high performance liquid chromatography (HPLC) as described in Pearson and Reanier, J. Chrom. 255:137-149 (1983).

### Definitions

Unless specifically indicated otherwise, all technical and scientific terms used herein have the same meaning as commonly understood by those of ordinary skill in the art to which this disclosure belongs. In addition, any method or material similar or equivalent to a method or material described herein can be used in the practice of the present disclosure. For purposes of the present disclosure, the following terms are defined.

The terms “a,” “an,” or “the” as used herein not only include aspects with one member, but also include aspects with more than one member. For instance, the singular forms “a,” “an,” and “the” include plural referents unless the context clearly dictates otherwise. Thus, for example, reference to “a cell” includes a plurality of such cells and reference to “the agent” includes reference to one or more agents known to those skilled in the art, and so forth.

The term “about” in relation to a reference numerical value can include a range of values plus or minus 10% from that value. For example, the amount “about 10” includes amounts from 9 to 11, including the reference numbers of 9, 10, and 11. The term “about” in relation to a reference numerical value can also include a range of values plus or minus 10%, 9%, 8%, 7%, 6%, 5%, 4%, 3%, 2%, or 1% from that value.

The term “nucleic acid,” “nucleotide,” or “polynucleotide” refers to deoxyribonucleic acids (DNA), ribonucleic acids (RNA) and polymers thereof in either single-, double- or multi-stranded form. The term includes, but is not limited to, single-, double- or multi-stranded DNA or RNA, genomic DNA, cDNA, DNA-RNA hybrids, or a polymer comprising purine and/or pyrimidine bases or other natural, chemically modified, biochemically modified, non-natural, synthetic or derivatized nucleotide bases. In some embodiments, a nucleic acid can comprise a mixture of DNA, RNA and analogs thereof. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, single nucleotide polymorphisms (SNPs), and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

The term “guide array” refers to a nucleic acid molecule comprising one or more, two, three or a plurality of sequences that encode an RNA molecule (e.g., a CRISPR-associated RNA or “crRNA”), where the RNA comprises a direct repeat stem loop sequence and a spacer sequence (e.g., a guide RNA) that can bind to an RNA target sequence via RNA-RNA hybridization. The term includes a DNA molecule or vector comprising one or more, two, three or multiple sequences that encode different crRNA molecules, and includes polycistronic and multicistronic DNA molecules encoding different crRNA molecules.

The terms “encode” or encodes” refer to a nucleic acid sequence comprising an open reading frame that can be transcribed by an RNA polymerase and translated into a polypeptide or protein of the disclosure (e.g., a class 2 type VI-D CRISPR effector). The term also includes nucleic acid sequences that are transcribed to produce an RNA that is not translated into a polypeptide or protein, such as a nucleic acid sequence encoding a guide array of the disclosure.

As used herein the term “non-targeting control guide” is a crRNA with a spacer that was randomly generated and BLASTED against the human transcriptome to ensure there were no matches to any known human RNA transcripts.

The term “gene” means the segment of DNA involved in producing a polypeptide chain. The DNA segment may include regions preceding and following the coding region (leader and trailer) involved in the transcription/translation of the gene product and the regulation of the transcription/translation, as well as intervening sequences (introns) between individual coding segments (exons).

The term “cassette” refers to a combination of genetic sequence elements that may be introduced as a single element and may function together to achieve a desired result. A cassette typically comprises polynucleotides in combinations that are not found in nature. A cassette can be inserted into a vector, such as an expression vector.

The term “operably linked” refers to two or more genetic sequence elements, such as a polynucleotide coding sequence and a promoter sequence, placed in relative positions in a polynucleotide, cassette or vector that permit the proper biological functioning of the elements, such as the promoter directing transcription of the coding sequence.

The term “inducible promoter” refers to a promoter that responds to environmental factors and/or external stimuli that can be artificially controlled in order to modify the expression of, or the level of expression of, a polynucleotide sequence or refers to a combination of elements, for example an exogenous promoter and an additional element such as a trans-activator operably linked to a separate promoter. An inducible promoter may respond to abiotic factors such as oxygen levels or to chemical or biological molecules. In some embodiments, the chemical or biological molecules may be molecules not naturally present in humans.

The terms “vector” and “expression vector” refer to a nucleic acid construct, generated recombinantly or synthetically, with a series of specified nucleic acid elements that permit transcription of a particular polynucleotide sequence in a host cell. An expression vector may be part of a plasmid, viral genome, or nucleic acid fragment. Typically, an expression vector includes a polynucleotide to be transcribed, operably linked to a promoter. The term “promoter” is used herein to refer to an array of nucleic acid control sequences that direct transcription of a nucleic acid. As used herein, a promoter includes necessary nucleic acid sequences near the start site of transcription, such as, in the case of a polymerase II type promoter, a TATA element. A promoter also optionally includes distal enhancer or repressor elements, which can be located as much as several thousand base pairs from the start site of transcription. Other elements that may be present in an expression vector include those that enhance transcription (e.g., enhancers) and terminate transcription (e.g., terminators).

“Recombinant” refers to a genetically modified polynucleotide, polypeptide, cell, tissue, or organism. For example, a recombinant polynucleotide (or a copy or complement of a recombinant polynucleotide) is one that has been manipulated using well known methods. A recombinant expression cassette comprising a promoter operably linked to a second polynucleotide (e.g., a coding sequence) can include a promoter that is heterologous to the second polynucleotide as the result of human manipulation (e.g., by methods described in Sambrook et al., Molecular Cloning-A Laboratory Manual, Cold Spring Harbor Laboratory, Cold Spring Harbor, New York, (1989) or Current Protocols in Molecular Biology Volumes 1-3, John Wiley & Sons, Inc (1994-1998)). A recombinant expression cassette (or expression vector) typically comprises polynucleotides in combinations that are not found in nature. For instance, human manipulated restriction sites or plasmid vector sequences can flank or separate the promoter from other sequences. A recombinant protein is one that is expressed from a recombinant polynucleotide, and recombinant cells, tissues, and organisms are those that comprise recombinant sequences (polynucleotide and/or polypeptide).

The terms “subject,” “individual,” and “patient” are used interchangeably herein to refer to a vertebrate, including a mammal or a human. Mammals include, but are not limited to, murines, simians, humans, farm animals, sport animals, and pets. Tissues, cells and their progeny of a biological entity obtained in vivo or cultured in vitro are also encompassed.

“Percent similarity” or “percent identity” in the context of polynucleotide or peptide sequences, is determined by comparing two optimally aligned sequences over a comparison window, wherein the portion of the sequence in the comparison window may comprise additions or deletions (i.e., gaps) as compared to the reference sequence which does not comprise additions or deletions, for optimal alignment of the two sequences. The percentage is calculated by determining the number of positions at which the identical nucleotide or amino acid occurs in both sequences to yield the number of matched positions, dividing the number of matched positions by the total number of positions in the window of comparison and multiplying the result by 100 to yield the percentage of similarity or identity (e.g., sequence similarity).

A polynucleotide or peptide is considered substantially similar when it comprises a sequence having at least, or greater than or equal to, about 60%, 65%, 70%, 75%, 80%, 85%, 86%, 87%, 88%, 89%, 90%, 91%, 92%, 93%, 94%, 95%, 96%, 97%, 98%, 99%, or 100% similarity, to a reference sequence, when compared and aligned for maximum correspondence over a comparison window, or designated region as measured using one of the following sequence comparison algorithms or by manual alignment and visual inspection. With regard to polynucleotide sequences, this definition also refers to the complement of a test sequence.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence similarities for the test sequences relative to the reference sequence, based on the program parameters. For sequence comparison of nucleic acids and proteins, the BLAST and BLAST 2.0 algorithms and the default parameters discussed below are used.

Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, WI), or by manual alignment and visual inspection (see, e.g., Current Protocols in Molecular Biology (Ausubel et al., eds. 1995 supplement)).

Additional examples of algorithms that are suitable for determining percent sequence similarity are the BLAST and BLAST 2.0 algorithms, which are described in Altschul et al., (1990). J. Mol. Biol. 215:403-410 and Altschul et al. (1977) Nucleic Acids Res. 25:3389-3402, respectively. Software for performing BLAST analyses is publicly available at the National Center for Biotechnology Information website, ncbi.nlm.nih.gov. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). The BLASTN program (for nucleotide sequences) uses as defaults a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix (see, e.g., Henikoff and Henikoff, Proc. Natl. Acad. Sci. USA 89:10915 (1989)).

The BLAST algorithm also performs a statistical analysis of the similarity between two sequences (see, e.g., Karlin and Altschul, Proc. Nat'l. Acad. Sci. USA, 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

As used herein, the term “administering” includes oral administration, topical contact, administration as a suppository, intravenous, intraperitoneal, intramuscular, intralesional, intrathecal, intranasal, or subcutaneous administration to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, and transdermal patches.

The term “treating” refers to an approach for obtaining beneficial or desired results including, but not limited to, a therapeutic benefit and/or a prophylactic benefit. By therapeutic benefit is meant any therapeutically relevant improvement in or effect on one or more diseases, conditions, or symptoms under treatment. For prophylactic benefit, the compositions of the disclosure may be administered to a subject at risk of developing a particular disease, condition, or symptom, or to a subject reporting one or more of the physiological symptoms of a disease, even though the disease, condition, or symptom may not have yet been manifested.

The term “effective amount” or “sufficient amount” refers to the amount of an agent that is sufficient to effect beneficial or desired results. The therapeutically effective amount may vary depending upon one or more of: the subject and disease condition being treated, the weight and age of the subject, the severity of the disease condition, the manner of administration and the like, which can readily be determined by one of ordinary skill in the art. The specific amount may vary depending on one or more of: the particular agent chosen, the host cell type, the location of the host cell in the subject, the dosing regimen to be followed, whether it is administered in combination with other compounds, timing of administration, and the physical delivery system in which it is carried.

The term “pharmaceutically acceptable carrier” refers to a substance that aids the administration of an active agent to a cell, an organism, or a subject. The term also refers to a carrier or excipient that can be included in the compositions of the disclosure and that causes no significant adverse toxicological effect on the patient. Non-limiting examples of pharmaceutically acceptable carrier include water, NaCl, normal saline solutions, lactated Ringer's, normal sucrose, normal glucose, cell culture media, and the like. One of skill in the art will recognize that other pharmaceutical carriers are useful in the present disclosure.

### Detailed Description of the Embodiments

### I. Genetically Modified T Cells

Provided herein are genetically modified T cells that express (i) a class 2 type VI-D CRISPR effector having RNA-guided RNA endonuclease activity, and (ii) a guide array comprising a plurality of CRISPR-associated RNA (crRNA) molecules. In some embodiments, the crRNA molecules independently comprise a direct repeat sequence and a spacer sequence that binds a target RNA. In some embodiments, the crRNA molecules bind to different target mRNAs. In some embodiments, the crRNA molecules bind to different regions of the same target mRNA.

In some embodiments, the modified T cell comprises a nucleic acid encoding a class 2 type VI-D CRISPR effector having RNA-guided RNA endonuclease activity, and/or a nucleic acid encoding or expressing a guide array comprising a plurality of CRISPR-associated RNA (crRNA) molecules. In some embodiments, the nucleic acid encoding (i) or the nucleic acid encoding or expressing (ii), or the nucleic acid encoding or expressing both (i) and (ii) are stably integrated into the genome of the T cell. In some embodiments, the nucleic acid encoding (i) or the nucleic acid encoding or expressing (ii), or the nucleic acid encoding or expressing both (i) and (ii) are not integrated into the genome of the T cell (e.g., transient transfection of the nucleic acids).

For a given class 2 type VI-D CRISPR effector, the direct repeat sequence typically comprises the same sequence for all crRNA molecules in the guide array. Thus, a guide array comprising N different crRNA molecules can comprise crRNA molecules having the same or substantially the same direct repeat sequence and N different spacer sequences. In some embodiments, the guide array comprises different spacer sequences that bind to different or distinct target mRNAs expressed by different target genes, for example 2 to N different target genes. In some embodiments, the guide array comprises different spacer sequences that bind to different sequences in the same target mRNA expressed by a given target gene (e.g., an mRNA expressed by the TOX gene). The spacer sequences can bind to distinct, separate, or non-overlapping sequences present in the same target mRNA or bind to partially overlapping sequences in the same target mRNA. In some embodiments, the guide array comprises a combination of i) spacer sequences that bind to different or distinct target mRNAs expressed by different target genes and ii) spacer sequences that bind to different sequences in the same target mRNA expressed by a given target gene.

In some embodiments, the guide array is a multicistronic array comprising a plurality of crRNA molecules. The guide array can comprise 2 or more crRNA molecules. In some embodiments, the guide array comprises 2 to 10 crRNA molecules. In some embodiments, the guide array comprises greater than 10 crRNA molecules. It will be understood that the number of crRNA molecules in a guide array is a function of the transcriptional elongation activity and/or processivity of the promoter used in the nucleic acid comprising the multicistronic array In embodiments where the promoter has increased transcriptional elongation activity and/or processivity, it is expected that longer guide arrays, e.g., comprising greater than 10 crRNA molecules, will be transcribed from the multicistronic array. In some embodiments, the promoter is an RNA polymerase (Pol) III promoter such as the U6 and H1 promoters, or modified versions thereof. In some embodiments, the promoter is an RNA polymerase II promoter.

In some embodiments, the class 2 type VI-D CRISPR effector having RNA-guided RNA endonuclease activity is a Cas13 nuclease selected from the group consisting of Cas13a, Cas13b, 13bt1, Cas13bt2, Cas13c, Cas13d, RfxCas13d, Cas13e, and functional variants or orthologs thereof. In some embodiments, the Cas13 nuclease comprises an amino acid sequence having at least (e.g., greater than or equal to) about 60%, 65%, 70%, 75%, 80%, 85%, 86%, 87%, 88%, 89%, 90%, 91%, 92%, 93%, 94%, 95%, 96%, 97%, 98%, 99%, or 100% sequence similarity or identity to the amino acid sequence of a nuclease selected from the group consisting of Cas13a, Cas13b, 13bt1, Cas13bt2, Cas13c, Cas13d, RfxCas13d, and Cas13e. In some embodiments, the Cas 13 nuclease comprises an amino acid sequence having at least (e.g., greater than or equal to) about 60%, 65%, 70%, 75%, 80%, 85%, 86%, 87%, 88%, 89%, 90%, 91%, 92%, 93%, 94%, 95%, 96%, 97%, 98%, 99%, or 100% sequence identity to SEQ ID NO:1.

In some embodiments, the class 2 type VI-D CRISPR effector having RNA-guided RNA endonuclease activity comprises a fusion protein comprising a destabilization domain (DD).

In some embodiments, the guide array comprises crRNA molecules that bind to an mRNA encoding a protein associated with T cell exhaustion. In some embodiments, the protein associated with T cell exhaustion is selected from the group consisting of TOX, TOX2, NR4A1, NR4A2, NR4A3, TET2, IRF4, JUNB, BATF3, DHX37, FLI1, ZC3H12A, SOCS1, TCEB2, PDCD1 (PD-1), HAVCR2 (TIM3), LAG3, CTLA4, TIGIT, FAS, TRAC, CBLB, RASA2, PTPN2, CD5, ENTPD1, CD46, B2M, and combinations thereof.

In some embodiments, the guide array comprises crRNA molecules that bind to an mRNA encoding a protein selected from the group consisting of CCNC, CDK8, CDK19, MED12, MED12L, MED13, MED13L, MED15, MED16, MED19, MED24, MED26, and combinations thereof.

### II. CAR-T Cells

In some embodiments, the modified T cell further comprises a chimeric antigen receptor (CAR). CAR T cells express a receptor that binds an antigen on a target cell, such as a tumor cell, and activates native T cell functionality to target and kill the target cell. First-generation CAR T cell therapeutic receptors comprise an extracellular antigen binding domain and an intracellular T cell activating domain. Second-generation CAR T cell therapeutic receptors include both a costimulatory domain and a T cell activation domain on their intracellular side. The costimulatory domain improves the therapeutic response of T cells by increasing T cell proliferation or cytotoxicity. In some embodiments, the CAR comprises an antigen binding scFv or nanobody. In some embodiments, the CAR comprises the intramembrane signaling domains CD28 and CD3G (CD247). In some embodiments, the CAR comprises the intramembrane signaling domains 4-1BB (CD137) and CD3ζ.

In some embodiments, the CAR binds to an antigen expressed by a tumor. In some embodiments, the CAR binds to an antigen selected from the group consisting of Her-2, B7-H3, GPC2, GD2, CD19, CD20, CD22, MAGE, BAGE, CAGE, GAGE, HAGE, LAGE, PAGE, PRAME, NY-ESO-1, NY-SEO-1, tyrosinase, Melan-A/MART, gp100, TRP-1, TRP-2, CD30, EGFR, EGFRvIII, FAP, CD33, CD123, PD-L1, IGF1R, CD4, CSPG4, B7-H4, NKG2D, CS1, CD138, EpCAM, EBNA3C, GPA7, CD244, CA-125, ETA, CEA, CD52, MUCSAC, c-Met, FAB, WT-1, PSMA, AFP, BCMA, Mesothelin, GPC3, MUC1 and CTAG1B.

As is known in the art, chronic intracellular signaling by a CAR can lead to T cell exhaustion, resulting in dysfunctional CAR T cells. Likewise, prolonged intracellular signaling by the endogenous T Cell Receptor (TCR) can also lead to T cell exhaustion, resulting in decreased anti-tumor activity. T cell exhaustion is characterized by increased expression of exhaustion-associated genes. The modified T cells of the disclosure can be used to decrease the expression of genes whose expression is increased during T cell exhaustion, thereby increasing the anti-tumor activity of the modified T cells. Thus, in some embodiments, intracellular signaling by the CAR or endogenous TCR upregulates T cell exhaustion markers in control T cells compared to the modified T cells of the disclosure. In some embodiments, the control T cell is a T cell that does not express (i) a class 2 type VI-D CRISPR effector having RNA-guided RNA endonuclease activity; and/or (ii) a guide array comprising a plurality of CRISPR-associated RNA (crRNA) molecules that bind to target RNAs expressed by exhaustion genes. In some embodiments, the control T cell is a CAR T cell that does not express (i) a class 2 type VI-D CRISPR effector having RNA-guided RNA endonuclease activity; and/or (ii) a guide array comprising a plurality of CRISPR-associated RNA (crRNA) molecules that bind to target RNAs expressed by exhaustion genes. In some embodiments, the control T cell is transfected with or expresses a non-targeting control guide array comprising crRNA molecules that do not bind to target RNAs.

In some embodiments, the exhaustion markers are selected from LAG3, PDCD1 (PD-1), HAVCR2 (TIM3), CTLA4 and ENTPD1 or a combination thereof. In some embodiments, the guide array comprises crRNA molecules that bind to mRNA encoding LAG3, PDCD1 (PD-1), HAVCR2 (TIM3), CTLA4 and ENTPD1 or a combination thereof.

In some embodiments, the modified T cells of the disclosure can include a “safety switch” for downregulating expression of a CAR expressed by the CAR T cell in a subject or patient undergoing CAR T cell therapy. Thus, in some embodiments, the guide array comprises crRNA molecules that bind to mRNA expressed by the CAR, thereby decreasing expression of the CAR. In some embodiments, the crRNA molecules bind to regions of the mRNA encoding the signaling elements of a CAR, such as the intramembrane signaling domains 4-1BB, CD28 and CD3G or endogenous TCR, such as ZAP70 and LCK.

In some embodiments, the T cell is a primary T cell isolated from a subject. In some embodiments, the T cell is a human T cell. In some embodiments, the T cell is a primary human T cell.

### III. Systems for Multiplexed Transcriptomic Regulation

Also provided herein are systems for multiplexed transcriptomic regulation. The systems can include (i) an expression cassette comprising a nucleic acid sequence encoding a class 2 type VI-D CRISPR effector having RNA-guided RNA endonuclease activity of the disclosure; and (ii) an expression cassette comprising a nucleic acid sequence encoding a guide array of the disclosure.

In some embodiments, the crRNA molecules comprise a direct repeat sequence and a spacer sequence that binds a target mRNA. In some embodiments, the crRNA molecules bind to different target mRNAs or different regions of the same target mRNA.

In some embodiments, the class 2 type VI-D CRISPR effector having RNA-guided RNA endonuclease activity is a Cas13 nuclease selected from the group consisting of Cas13a, Cas13b, 13bt1, Cas13bt2, Cas13c, Cas13d, RfxCas13d, Cas13e, and functional variants or orthologs thereof. In some embodiments, the Cas13 nuclease comprises an amino acid sequence having at least (e.g., greater than or equal to) about 60%, 65%, 70%, 75%, 80%, 85%, 86%, 87%, 88%, 89%, 90%, 91%, 92%, 93%, 94%, 95%, 96%, 97%, 98%, 99%, or 100% sequence similarity or identity to the amino acid sequence of a nuclease selected from the group consisting of Cas13a, Cas13b, 13bt1, Cas13bt2, Cas13c, Cas13d, RfxCas13d, and Cas13e. In some embodiments, the Cas 13 nuclease comprises an amino acid sequence having at least (e.g., greater than or equal to) about 60%, 65%, 70%, 75%, 80%, 85%, 86%, 87%, 88%, 89%, 90%, 91%, 92%, 93%, 94%, 95%, 96%, 97%, 98%, 99%, or 100% sequence identity to SEQ ID NO: 1.

In some embodiments, the class 2 type VI-D CRISPR effector having RNA-guided RNA endonuclease activity comprises a fusion protein comprising a destabilization domain (DD).

In some embodiments, the guide array comprises crRNA molecules that bind to an mRNA encoding a protein associated with T cell exhaustion. In some embodiments, the protein associated with T cell exhaustion is selected from the group consisting of TOX, TOX2, NR4A1, NR4A2, NR4A3, TET2, IRF4, JUNB, BATF3, DHX37, FLI1, ZC3H12A, SOCS1, TCEB2, PDCD1 (PD-1), HAVCR2 (TIM3), LAG3, CTLA4, TIGIT, FAS, TRAC, CBLB, RASA2, PTPN2, CD5, ENTPD1, CD46, B2M, and combinations thereof.

In some embodiments, the systems can further comprise a T cell or primary T cell. In some embodiments, the T cell is a primary T cell isolated from a subject. In some embodiments, the T cell is a human T cell. In some embodiments, the T cell is a primary human T cell. In some embodiments, the T cell is transformed or transduced with an expression cassette of the system. In some embodiments, the T cell is transformed or transduced with an expression cassette of (i) and (ii) above, such that the T cell expresses a class 2 type VI-D CRISPR effector having RNA-guided RNA endonuclease activity of the disclosure; and a guide array of the disclosure.

In some embodiments, the T cell is a modified T cell comprising a chimeric antigen receptor (CAR). In some embodiments, the CAR binds to an antigen expressed by a tumor. In some embodiments, the CAR binds to an antigen selected from the group consisting of Her-2, B7-H3, GPC2, GD2, CD19, CD20, CD22, MAGE, BAGE, CAGE, GAGE, HAGE, LAGE, PAGE, PRAME, NY-ESO-1, NY-SEO-1, tyrosinase, Melan-A/MART, gp100, TRP-1, TRP-2, CD30, EGFR, EGFRvIII, FAP, CD33, CD123, PD-L1, IGF1R, CD4, CSPG4, B7-H4, NKG2D, CSI, CD138, EpCAM, EBNA3C, GPA7, CD244, CA-125, ETA, CEA, CD52, MUCSAC, c-Met, FAB, WT-1, PSMA, AFP, BCMA, Mesothelin, GPC3, MUC1 and CTAG1B.

In some embodiments, intracellular signaling by the CAR or endogenous TCR upregulates T cell exhaustion markers in a control T cell. In some embodiments, the control T cell is a T cell that does not express (i) a class 2 type VI-D CRISPR effector having RNA-guided RNA endonuclease activity; and/or (ii) a guide array comprising a plurality of CRISPR-associated RNA (crRNA) molecules. In some embodiments, the control T cell is a CAR T cell that does not express (i) a class 2 type VI-D CRISPR effector having RNA-guided RNA endonuclease activity; and/or (ii) a guide array comprising a plurality of CRISPR-associated RNA (crRNA) molecules. In some embodiments, the control T cell is transfected with or expresses a non-targeting control guide array comprising crRNA molecules that do not bind to target RNAs.

In some embodiments, the exhaustion markers are selected from LAG3, PDCD1 (PD-1), HAVCR2 (TIM3), CTLA4 and ENTPD1 or a combination thereof. In some embodiments, the guide array comprises crRNA molecules that bind to mRNA encoding LAG3, PDCD1 (PD-1), HAVCR2 (TIM3), CTLA4 and ENTPD1 or a combination thereof.

In some embodiments, the T cell includes a “safety switch” for downregulating expression of a CAR expressed by the CAR T cell in a subject or patient undergoing CAR T cell therapy. Thus, in some embodiments, the guide array comprises crRNA molecules that bind to mRNA expressed by the CAR, thereby decreasing expression of the CAR. In some embodiments, the crRNA molecules bind to regions of the mRNA encoding the signaling elements of a CAR, such as the intramembrane signaling domains 4-1BB, CD28 and CD3G, or endogenous TCR, such as ZAP70 and LCK.

In some embodiments, the T cell is a primary T cell isolated from a subject. In some embodiments, the T cell is a human T cell. In some embodiments, the T cell is a primary human T cell.

### IV. Fusion Proteins

In some aspects, the class 2 type VI-D CRISPR effector having RNA-guided RNA endonuclease activity comprises a fusion protein comprising a destabilization domain (DD). In some embodiments, the DD comprises an E. coli dihydrofolate reductase DD linked to the C-terminus of the class 2 type VI-D CRISPR effector having RNA-guided RNA endonuclease activity.

In some embodiments, the class 2 type VI-D CRISPR effector having RNA-guided RNA endonuclease activity is a Cas13 nuclease selected from the group consisting of Cas13a, Cas13b, 13bt1, Cas13bt2, Cas13c, Cas13d, RfxCas13d, Cas13e, and functional variants or orthologs thereof. In some embodiments, the Cas13 nuclease comprises an amino acid sequence having at least (e.g., greater than or equal to) about 60%, 65%, 70%, 75%, 80%, 85%, 86%, 87%, 88%, 89%, 90%, 91%, 92%, 93%, 94%, 95%, 96%, 97%, 98%, 99%, or 100% sequence similarity or identity to the amino acid sequence of a nuclease selected from the group consisting of Cas13a, Cas13b, 13bt1, Cas13bt2, Cas13c, Cas13d, RfxCas13d, and Cas13e. In some embodiments, the Cas 13 nuclease comprises an amino acid sequence having at least (e.g., greater than or equal to) about 60%, 65%, 70%, 75%, 80%, 85%, 86%, 87%, 88%, 89%, 90%, 91%, 92%, 93%, 94%, 95%, 96%, 97%, 98%, 99%, or 100% sequence identity to SEQ ID NO: 1.

In some embodiments, the DD domain comprises an amino acid sequence having at least (e.g., greater than or equal to) about 60%, 65%, 70%, 75%, 80%, 85%, 86%, 87%, 88%, 89%, 90%, 91%, 92%, 93%, 94%, 95%, 96%, 97%, 98%, 99%, or 100% sequence similarity or identity to SEQ ID NO:5.

In some embodiments, the fusion protein comprises a linker sequence linking the class 2 type VI-D CRISPR effector and the DD. In some embodiments, the linker sequence comprises APKKKRKVGGSPAAKRVKLD (SEQ ID NO:11).

### V. Nucleic Acids and Vectors

Also provided are nucleic acids that encode a class 2 type VI-D CRISPR effector having RNA-guided RNA endonuclease activity, a guide array, a CAR, and/or a fusion protein of the disclosure. The nucleic acids can include plasmids and vectors comprising a nucleic acid encoding i) any class 2 type VI-D CRISPR effector having RNA-guided RNA endonuclease activity disclosed herein, ii) any CAR disclosed herein, iii) a guide array disclosed herein, and/or iv) a fusion protein of the disclosure. In some embodiments, the class 2 type VI-D CRISPR effector having RNA-guided RNA endonuclease activity is a Cas13 nuclease selected from the group consisting of Cas13a, Cas13b, 13bt1, Cas13bt2, Cas13c, Cas13d, RfxCas13d, Cas13e, and functional variants thereof. In some embodiments, the guide array is a multicistronic array comprising the plurality of crRNA moleculesIn some embodimentsthe guide array comprises from 2 to greater than or equal to 10 crRNA molecules. In some embodiments, the guide array comprises crRNA molecules that bind to mRNA encoding a protein associated with T cell exhaustion. In some embodiments, the protein associated with T cell exhaustion is selected from the group consisting of TOX, TOX2, NR4A1, NR4A2, NR4A3, TET2, IRF4, JUNB, BATF3, DHX37, FLI1, ZC3H12A, SOCS1, TCEB2, PDCD1 (PD-1), HAVCR2 (TIM3), LAG3, CTLA4, TIGIT, FAS, TRAC, CBLB, RASA2, PTPN2, CD5, ENTPD1, CD46, B2M, and combinations thereof.

In some embodiments, the vector is an expression vector or comprises an expression cassette. In some embodiments, the vector comprises sequences that regulate transcription, translation and/or RNA stability, such as an enhancer, a 5′ UTR, a promoter, a polyA sequence, a 3′ UTR, and/or a nuclear localization sequence. It will be understood that the sequences that regulate transcription, translation and/or RNA stability can be operably linked to other sequences in the vector. For example, a promoter can be operably linked to an open reading frame encoding a class 2 type VI-D CRISPR effector having RNA-guided RNA endonuclease activity, a CAR, and/or a fusion protein of the disclosure. In some embodiments, the promoter is operably link to nucleic acids that express a guide array disclosed herein. In some embodiments the promoter is a constitutive promoter. In some embodiments the promoter is an inducible promoter.

In some embodiments, the vector is a viral vector. The viral vector can be any viral vector suitable for delivering nucleic acids to cells. The vector can be, for example, an adeno-associated virus (AAV) vector, an adenovirus vector, a retrovirus vector, a lentivirus vector, or a herpes simplex virus (HSV) vector. In some embodiments, the vector is a lentiviral vector.

### VI. Guide Arrays

Also provided are guide arrays. Guide arrays comprise nucleic acids (polynucleotides) comprising a direct repeat sequence and a spacer sequence. Guide arrays are typically expressed from a plasmid or vector comprising a promoter operably linked to the guide array. The direct repeat sequence can form a stem-loop structure, whereas the spacer sequence comprises sequences complementary to a target RNA in the cell. The spacer sequence binds to the target RNA by RNA-RNA hybridization. Exemplary non-limiting spacer sequences are provided in Tables 1 and 2.

In some embodiments, the guide array is a multicistronic array comprising a plurality of crRNA molecules. The guide array can comprise 2 or more crRNA molecules. In some embodiments, the guide array comprises 2 to 10 crRNA molecules. In some embodiments, the guide array comprises greater than 10 crRNA molecules. It will be understood that the number of crRNA molecules in a guide array is a function of the transcriptional elongation activity and/or processivity of the promoter used in the nucleic acid comprising the multicistronic array. In embodiments where the promoter has increased transcriptional elongation activity and/or processivity, it is expected that longer guide arrays, e.g., comprising greater than 10 crRNA molecules, will be transcribed from the multicistronic array. In some embodiments, the promoter is an RNA polymerase (Pol) III promoter such as the U6 and H1 promoters, or modified versions thereof. In some embodiments, the promoter is an RNA polymerase II promoter.

As described above, the direct repeat sequence typically comprises the same sequence for all crRNA molecules in the guide array. Thus, a guide array comprising N different crRNA molecules can comprise crRNA molecules having the same or substantially the same direct repeat sequence and N different spacer sequences. In some embodiments, the guide array comprises different spacer sequences that bind to different or distinct target mRNAs expressed by different target genes, for example 2 to N different target genes. In some embodiments, the guide array comprises different spacer sequences that bind to different sequences in the same target mRNA expressed by a given target gene (e.g., an mRNA expressed by the TOX gene). The spacer sequences can bind to distinct, separate, or non-overlapping sequences present in the same target mRNA or bind to partially overlapping sequences in the same target mRNA. In some embodiments, the guide array comprises a combination of i) spacer sequences that bind to different or distinct target mRNAs expressed by different target genes and ii) spacer sequences that bind to different sequences in the same target mRNA expressed by a given target gene.

In some embodiments, the direct repeat sequence comprises AACCCCTACCAACTGGTCGGGGTTTGAAAC (SEQ ID NO:9). In some embodiments, the direct repeat sequence comprises CAAGTAAACCCCTACCAACTGGTCGGGGTTTGAAAC (SEQ ID NO:10).

### VII. Methods for Producing Modified T Cells

Also provided are method for producing a modified T cell that expresses a class 2 type VI-D CRISPR effector and a guide array of the disclosure. The methods can comprise transfecting or transducing a T cell with one or more expression vectors that encode a class 2 type VI-D CRISPR effector having RNA-guided RNA endonuclease activity and/or expresses a guide array comprising one or more, or a plurality of CRISPR-associated RNA (crRNA) molecules.

In some embodiments, the T cell is transfected or transduced with two separate vectors, a first vector comprising a nucleic acid sequence encoding a class 2 type VI-D CRISPR effector having RNA-guided RNA endonuclease activity, and a second vector comprising a nucleic acid sequence encoding a guide array comprising a plurality of CRISPR-associated RNA (crRNA) molecules. In some embodiments, co-transduction of T cells with two separate vectors increases the targeting efficiency when compared to transduction with a single vector that expresses both the class 2 type VI-D CRISPR effector and the guide array. In some embodiments, the T cell is transduced with a first vector that expresses the class 2 type VI-D CRISPR effector before transduction with a second vector comprising the guide array. In some embodiments, the T cell is transduced with a first vector that expresses the class 2 type VI-D CRISPR effector about 12 to 36 hours before transduction with a second vector comprising the guide array.

In some embodiments, the guide array is a multicistronic array comprising a plurality of crRNA molecules. The guide array can comprise 2 or more crRNA molecules. In some embodiments, the guide array comprises 2 to 10 crRNA molecules. In some embodiments, the guide array comprises greater than 10 crRNA molecules. It will be understood that the number of crRNA molecules in a guide array is a function of the transcriptional elongation activity and/or processivity of the promoter used in the nucleic acid comprising the multicistronic array. In embodiments where the promoter has increased transcriptional elongation activity and/or processivity, it is expected that longer guide arrays, e.g., comprising greater than 10 crRNA molecules, will be transcribed from the multicistronic array. In some embodiments, the promoter is an RNA polymerase (Pol) III promoter such as the U6 and HI promoters, or modified versions thereof. In some embodiments, the promoter is an RNA polymerase II promoter.

In some embodiments, the class 2 type VI-D CRISPR effector having RNA-guided RNA endonuclease activity is a Cas13 nuclease selected from the group consisting of Cas13a, Cas13b, 13bt1, Cas13bt2, Cas13c, Cas13d, RfxCas13d, Cas13e, and functional variants or orthologs thereof. In some embodiments, the Cas13 nuclease comprises an amino acid sequence having at least (e.g., greater than or equal to) about 60%, 65%, 70%, 75%, 80%, 85%, 86%, 87%, 88%, 89%, 90%, 91%, 92%, 93%, 94%, 95%, 96%, 97%, 98%, 99%, or 100% sequence similarity or identity to the amino acid sequence of a nuclease selected from the group consisting of Cas13a, Cas13b, 13bt1, Cas13bt2, Cas13c, Cas13d, RfxCas13d, and Cas13e. In some embodiments, the Cas 13 nuclease comprises an amino acid sequence having at least (e.g., greater than or equal to) about 60%, 65%, 70%, 75%, 80%, 85%, 86%, 87%, 88%, 89%, 90%, 91%, 92%, 93%, 94%, 95%, 96%, 97%, 98%, 99%, or 100% sequence identity to SEQ ID NO: 1.

In some embodiments, the guide array comprises crRNA molecules that bind to an mRNA encoding a protein associated with T cell exhaustion. In some embodiments, the protein associated with T cell exhaustion is selected from the group consisting of TOX, TOX2, NR4A1, NR4A2, NR4A3, TET2, IRF4, JUNB, BATF3, DHX37, FLI1, ZC3H12A, SOCS1, TCEB2, PDCD1 (PD-1), HAVCR2 (TIM3), LAG3, CTLA4, TIGIT, FAS, TRAC, CBLB, RASA2, PTPN2, CD5, ENTPD1, CD46, B2M, and combinations thereof.

In some embodiments, the T cells is further or optionally transduced with a third vector comprising a nucleic acid sequence encoding a CAR. The T cell can be transduced with a first vector encoding a class 2 type VI-D CRISPR effector of the disclosure and a third vector encoding a CAR at about the same time, e.g., simultaneously or contemporaneously, or within minutes or several hours (e.g., 1 to 8 hours) of each other. In some embodiments, the T cell is transduced with the first and third vectors and then transformed with the second vector comprising the guide array 12 to 36 hours later.

In some embodiments, the first, second, and/or third vectors are stably integrated into the genome of the host T cell. In some embodiments, the first, second, and/or third vectors are not integrated into the genome of the T cell (e.g., transient transfection of the vectors).

In some embodiments, the CAR binds to an antigen expressed by a tumor. In some embodiments, the CAR binds to an antigen selected from the group consisting of Her-2, B7-H3, GPC2, GD2, CD19, CD20, CD22, MAGE, BAGE, CAGE, GAGE, HAGE, LAGE, PAGE, PRAME, NY-ESO-1, NY-SEO-1, tyrosinase, Melan-A/MART, gp100, TRP-1, TRP-2, CD30, EGFR, EGFRvIII, FAP, CD33, CD123, PD-L1, IGF1R, CD4, CSPG4, B7-H4, NKG2D, CS1, CD138, EpCAM, EBNA3C, GPA7, CD244, CA-125, ETA, CEA, CD52, MUC5AC, c-Met, FAB, WT-1, PSMA, AFP, BCMA, Mesothelin, GPC3, MUC1 and CTAG1B.

In some embodiments, the CAR binds to a tumor antigen described herein.

In some embodiments, intracellular signaling by the CAR or endogenous TCR upregulates T cell exhaustion markers in a control T cell. In some embodiments, the control T cell is a T cell that does not express (i) a class 2 type VI-D CRISPR effector having RNA-guided RNA endonuclease activity; and/or (ii) a guide array comprising a plurality of CRISPR-associated RNA (crRNA) molecules that bind to RNAs expressed by exhaustion genes. In some embodiments, the control T cell is a CAR T cell that does not express (i) a class 2 type VI-D CRISPR effector having RNA-guided RNA endonuclease activity; and/or (ii) a guide array comprising a plurality of CRISPR-associated RNA (crRNA) molecules that bind to RNAs expressed by exhaustion genes. In some embodiments, the control T cell is transfected with or expresses a non-targeting control guide array comprising crRNA molecules that do not bind to target RNAs.

In some embodiments, the exhaustion markers are selected from LAG3, PDCD1 (PD-1), HAVCR2 (TIM3), CTLA4 and ENTPD1 or a combination thereof. In some embodiments, the guide array comprises crRNA molecules that bind to mRNA encoding LAG3, PDCD1 (PD-1), HAVCR2 (TIM3), CTLA4 and ENTPD1 or a combination thereof.

In some embodiments, the T cell includes a “safety switch” for downregulating expression of a CAR expressed by the CAR T cell in a subject or patient undergoing CAR T cell therapy. Thus, in some embodiments, the guide array comprises crRNA molecules that bind to mRNA expressed by the CAR, thereby decreasing expression of the CAR. In some embodiments, the crRNA molecules bind to regions of the mRNA encoding the signaling elements of a CAR, such as the intramembrane signaling domains 4-1BB, CD28 and CD36, or endogenous TCR, such as ZAP70 and LCK.

### VIII. Methods for Regulating Gene Expression in T Cells

In some aspects, the disclosure provides a method for regulating gene expression in a T cell. The methods provide the advantage of tunable and reversible control of the T cell transcriptome. In some embodiments, the T cell expresses a fusion protein of the disclosure and a guide array comprising a crRNA molecule, wherein the crRNA molecule comprises a direct repeat sequence and a spacer sequence that binds a target mRNA. In some embodiments, the fusion protein comprises a class 2 type VI-D CRISPR effector having RNA-guided RNA endonuclease activity and a destabilization domain (DD). In some embodiments, DD comprises an E. coli dihydrofolate reductase DD linked to the C-terminus of the class 2 type VI-D CRISPR effector having RNA-guided RNA endonuclease activity.

At steady-state, the class 2 type VI-D CRISPR effector-DD fusion protein is rapidly degraded by the proteasome due to the disordered DD. Thus, in some embodiments, the fusion protein is degraded in the T cell in the absence of the compound. In the presence of a suitable compound, tertiary structure of the DD is stabilized, allowing the class 2 type VI-D CRISPR effector-DD fusion protein to bind to and target RNA. Further, the inventors found that the T cells expressing the fusion protein and a guide RNA cultured in the presence of the compound could conditionally repress expression of a target gene, whereas expression of the target gene was not inhibited in the absence of the compound. Thus, in some embodiments, expression of a target gene is decreased in the presence of the compound compared to expression of the target gene in the absence of the compound.

The inventors also found that expression of a target gene in a T cell could be regulated by controlling the dose of a compound that stabilizes the DD of the fusion protein. Regulation of target gene expression was observed with a sigmoidal dose response curve between approximately 1 to 100 nM, as described in the Examples and FIG. 4. Thus, in some embodiments, expression of one or more target gene(s) is regulated by the compound in a dose-dependent manner.

The inventors further found that repression of target gene expression in T cells that expressed the fusion protein and guide RNA that binds to a target mRNA could be reversed by removing the compound that stabilizes the DD of the fusion protein from the culture medium, such that removal of the compound from the culture medium resulted in a rapid increase in target gene expression. Thus, in some embodiments, expression of the target gene is increased in T cells after removal of the compound, thereby reversibly regulating expression of the target gene.

In some embodiments, the method comprises: (a) transducing a T cell with (i) an expression vector comprising a nucleic acid sequence encoding a fusion protein of the disclosure; and (ii) an expression vector comprising a nucleic acid sequence encoding a guide array comprising a crRNA molecule, wherein the crRNA molecule comprises a direct repeat sequence and a spacer sequence that binds a target mRNA; (b) contacting the T cell with a compound that binds to and stabilizes the DD, wherein expression of a target gene is decreased in the presence of the compound compared to expression of the target gene in the absence of the compound.

In some embodiments, the compound is trimethoprim (TMP).

In some embodiments, the class 2 type VI-D CRISPR effector having RNA-guided RNA endonuclease activity is a Cas13 nuclease selected from the group consisting of Cas13a, Cas13b, 13bt1, Cas13bt2, Cas13c, Cas13d, RfxCas13d, Cas13e, and functional variants or orthologs thereof. In some embodiments, the Cas13 nuclease comprises an amino acid sequence having at least (e g., greater than or equal to) about 60%, 65%, 70%, 75%, 80%, 85%, 86%, 87%, 88%, 89%, 90%, 91%, 92%, 93%, 94%, 95%, 96%, 97%, 98%, 99%, or 100% sequence similarity or identity to the amino acid sequence of a nuclease selected from the group consisting of Cas13a, Cas13b, 13bt1, Cas13bt2, Cas13c, Cas13d, RfxCas13d, and Cas13e. In some embodiments, the Cas 13 nuclease comprises an amino acid sequence having at least (e.g., greater than or equal to) about 60%, 65%, 70%, 75%, 80%, 85%, 86%, 87%, 88%, 89%, 90%, 91%, 92%, 93%, 94%, 95%, 96%, 97%, 98%, 99%, or 100% sequence identity to SEQ ID NO: 1.

In some embodiments, the guide array comprises one or more or a plurality of crRNA molecules that bind to different target mRNAs or different regions of the same target mRNA. In some embodiments, the method can regulate the expression of more than one target gene in a T cell. In some embodiments, the method can simultaneously repress the expression of 1, 2, 3, 4, 5, 6, 7, 8, 9, 10 or more target genes. In some embodiments, the method can simultaneously repress the expression of one or more target genes associated with exhaustion in T cells. In some embodiments, the method can simultaneously repress the expression of a protein selected from the group consisting of TOX, TOX2, NR4A1, NR4A2, NR4A3, TET2, IRF4, JUNB, BATF3, DHX37, FLI1, ZC3H12A, SOCS1, TCEB2, PDCD1 (PD-1), HAVCR2 (TIM3), LAG3, CTLA4, TIGIT, FAS, TRAC, CBLB, RASA2, PTPN2, CD5, ENTPD1, CD46, B2M, and combinations thereof.

### IX. Methods for Screening to Identify Regulators of T Cell Activity

In another aspect, the disclosure provides a method for screening to identify regulators of T cell activity. The methods allow systematic genetic perturbations in primary T cells. In some embodiments, the method comprises expressing in a T cell or population of T cells:
(i) a class 2 type VI-D CRISPR effector having RNA-guided RNA endonuclease activity; and
(ii) a library of guide arrays, where individual guide arrays comprise one or more CRISPR-associated RNA (crRNA) molecules, wherein the crRNA molecules comprise a direct repeat sequence and a spacer sequence that binds a target RNA, and wherein the one or more crRNA molecules bind to different target mRNAs or different regions of the same target mRNA;
culturing the T cell(s) to produce a clonal population of expanded T cells; and
determining if a guide array is enriched or depleted in the clonal population of expanded T cells,
wherein if a guide array is enriched, then the target mRNA encodes a negative regulator of T cell activity, or if a guide array is depleted, then the target mRNA encodes a positive regulator of T cell activity.

In some embodiments, the T cell activity is T cell proliferation, increased cytokine secretion or increased tumor cell killing.

In some embodiments, T cells comprising an enriched guide array have an effector memory phenotype, and T cells comprising a depleted guide array have a central memory or stem cell memory phenotype. In some embodiments, determining if a guide array is enriched or depleted in the clonal population of expanded T cells comprises sequencing the guide RNAs present in the T cells.

In some embodiments, the individual guide arrays in the library comprise crRNA molecules that bind to an mRNA encoding a protein associated with T cell exhaustion. In some embodiments, the protein associated with T cell exhaustion is selected from the group consisting of TOX, TOX2, NR4A1, NR4A2, NR4A3, TET2, IRF4, JUNB, BATF3, DHX37, FLI1, ZC3H12A, SOCS1, TCEB2, PDCD1 (PD-1), HAVCR2 (TIM3), LAG3, CTLA4, TIGIT, FAS, TRAC, CBLB, RASA2, PTPN2, and combinations thereof.

In some embodiments, the library of guide arrays comprises one or more individual guide arrays comprising multicistronic arrays comprising a plurality of crRNA molecules. In some embodiments, the individual guide arrays comprise from 2 to 10 crRNA molecules. In some embodiments, the individual guide arrays comprise a pair of crRNA molecules.

In some embodiments, the class 2 type VI-D CRISPR effector having RNA-guided RNA endonuclease activity is a Cas13 nuclease selected from the group consisting of Cas13a, Cas13b, 13bt1, Cas13bt2, Cas13c, Cas13d, RfxCas13d, Cas13e, and functional variants thereof. In some embodiments, the Cas13 nuclease comprises an amino acid sequence having at least (e.g., greater than or equal to) about 60%, 65%, 70%, 75%, 80%, 85%, 86%, 87%, 88%, 89%, 90%, 91%, 92%, 93%, 94%, 95%, 96%, 97%, 98%, 99%, or 100% sequence similarity or identity to the amino acid sequence of a nuclease selected from the group consisting of Cas13a, Cas13b, 13bt1, Cas13bt2, Cas13c, Cas13d, RfxCas13d, and Cas13e. In some embodiments, the Cas13 nuclease comprises an amino acid sequence having at least (e.g., greater than or equal to) about 60%, 65%, 70%, 75%, 80%, 85%, 86%, 87%, 88%, 89%, 90%, 91%, 92%, 93%, 94%, 95%, 96%, 97%, 98%, 99%, or 100% sequence identity to SEQ ID NO: 1.

In some embodiments, the T cell further expresses a chimeric antigen receptor (CAR). Expression of the CAR can result in tonic signaling that results in both T cell expansion and subsequent dysfunction.

In some embodiments, the CAR binds to an antigen expressed by a tumor. In some embodiments, the CAR binds to an antigen selected from the group consisting of Her-2, B7-H3, GPC2, GD2, CD19, CD20, CD22, MAGE, BAGE, CAGE, GAGE, HAGE, LAGE, PAGE, PRAME, NY-ESO-1, NY-SEO-1, tyrosinase, Melan-A/MART, gp100, TRP-1, TRP-2, CD30, EGFR, EGFRvIII, FAP, CD33, CD123, PD-L1, IGF1R, CD4, CSPG4, B7-H4, NKG2D, CSI, CD138, EpCAM, EBNA3C, GPA7, CD244, CA-125, ETA, CEA, CD52, MUCSAC, c-Met, FAB, WT-1, PSMA, AFP, BCMA, Mesothelin, GPC3, MUC1 and CTAG1B.

### X. Method for Increasing Proliferation of T Cells

In another aspect, the disclosure provides a method for increasing the proliferation of a T cell. The methods allow systematic genetic perturbations in primary T cells to identify novel combinations of putative exhaustion-related genes that regulate the proliferation of dysfunctional T cells.

In some embodiments, the method comprises transducing a T cell with
(i) an expression vector comprising a nucleic acid sequence encoding a class 2 type VI-D CRISPR effector having RNA-guided RNA endonuclease activity; and
(ii) an expression vector comprising a nucleic acid sequence encoding a guide array comprising one or more crRNA molecules, wherein the crRNA molecules independently comprise a direct repeat sequence and a spacer sequence that binds a target mRNA, and wherein the crRNA molecules bind to different target mRNAs or different regions of the same target mRNA,
wherein proliferation of the T cell is increased compared to a control T cell that expresses a non-targeting control guide array comprising one or more crRNA molecules that do not bind to a target mRNA in (ii).

In some embodiments, the guide array comprises crRNA molecules that bind to an mRNA encoding a protein associated with T cell exhaustion. In some embodiments, the protein associated with T cell exhaustion is selected from the group consisting of FAS, ZC3H12A, CTLA4, SOCS1, CBLB, PD-1, TOX, TOX2, FLI1, CD5, CD39, CD46, TRAC, B2M, JUNB, IRF4, PDCD1 (PD-1), HAVCR2 (TIM3), DHX37, and combinations thereof.

In some embodiments, the guide array is, or is included in, a library of guide arrays, wherein the library of guide arrays comprises one or more individual guide arrays. The individual guide arrays can comprise multicistronic arrays comprising one or more, or a plurality of crRNA molecules. In some embodiments, the guide array comprises a multicistronic array comprising a plurality of crRNA molecules. In some embodiments, the guide array comprises from 2 to greater than or equal to 10 crRNA molecules. In some embodiments, the individual guide arrays comprise a pair (2) of crRNA molecules.

In some embodiments, the class 2 type VI-D CRISPR effector having RNA-guided RNA endonuclease activity is a Cas13 nuclease selected from the group consisting of Cas13a, Cas13b, 13bt1, Cas13bt2, Cas13c, Cas13d, RfxCas13d, Cas13e, and functional variants thereof. In some embodiments, the Cas13 nuclease comprises an amino acid sequence having at least (e.g., greater than or equal to) about 60%, 65%, 70%, 75%, 80%, 85%, 86%, 87%, 88%, 89%, 90%, 91%, 92%, 93%, 94%, 95%, 96%, 97%, 98%, 99%, or 100% sequence similarity or identity to the amino acid sequence of a nuclease selected from the group consisting of Cas13a, Cas13b, 13bt1, Cas13bt2, Cas13c, Cas13d, RfxCas13d, and Cas13e. In some embodiments, the Cas13 nuclease comprises an amino acid sequence having at least (e.g., greater than or equal to) about 60%, 65%, 70%, 75%, 80%, 85%, 86%, 87%, 88%, 89%, 90%, 91%, 92%, 93%, 94%, 95%, 96%, 97%, 98%, 99%, or 100% sequence identity to SEQ ID NO: 1.

In some embodiments, the method further comprises (iii) transducing the T cell with an expression vector comprising a nucleic acid sequence encoding a CAR. In some embodiments, the CAR binds to an antigen expressed by a tumor. In some embodiments, the CAR binds to an antigen selected from the group consisting of Her-2, B7-H3, GPC2, GD2, CD19, CD20, CD22, MAGE, BAGE, CAGE, GAGE, HAGE, LAGE, PAGE, PRAME, NY-ESO-1, NY-SEO-1, tyrosinase, Melan-A/MART, gpl00, TRP-1, TRP-2, CD30, EGFR, FAP, CD33, CD123, PD-L1, IGF1R, CD4, CSPG4, B7-H4, NKG2D, CSI, CD138, EpCAM, EBNA3C, GPA7, CD244, CA-125, ETA, CEA, CD52, MUCSAC, c-Met, EGFR, FAB, WT-1, PSMA, AFP, CEA, and CTAG1B.

### XI. Methods for Treating Tumors

The disclosure also provides methods for treating cancer or a tumor in a subject or patient in need of treatment. In some embodiments, the method comprises administering an effective amount of a genetically modified T cell of the disclosure to the subject, wherein the modified T cell kills tumor cells in the subject, thereby treating the tumor In some embodiments, the modified T cell expresses or comprises (i) a class 2 type VI-D CRISPR effector having RNA-guided RNA endonuclease activity of the disclosure, (ii) a guide array of the disclosure, or (iii) both (i) and (ii).

In some embodiments, the modified T cell comprises (i) an expression vector comprising a nucleic acid sequence encoding a class 2 type VI-D CRISPR effector having RNA-guided RNA endonuclease activity; and (ii) an expression vector comprising a nucleic acid sequence encoding a guide array comprising one or more crRNA molecules, wherein the crRNA molecules independently comprise a direct repeat sequence and a spacer sequence that binds a target mRNA, and wherein the crRNA molecules bind to different target mRNAs or different regions of the same target mRNA.

In some embodiments, the guide array comprises crRNA molecules that bind to an mRNA encoding a protein associated with T cell exhaustion. In some embodiments, the protein associated with T cell exhaustion is selected from the group consisting of FAS, ZC3H12A, CTLA4, SOCS1, CBLB, PD-1, TOX, TOX2, FLI1, CD5, CD39, CD46, TRAC, B2M, JUNB, IRF4, PDCD1 (PD-1), HAVCR2 (TIM3), DHX37, and combinations thereof.

In some embodiments, the class 2 type VI-D CRISPR effector having RNA-guided RNA endonuclease activity is a Cas13 nuclease selected from the group consisting of Cas13a, Cas13b, 13bt1, Cas13bt2, Cas13c, Cas13d, RfxCas13d, Cas13e, and functional variants thereof. In some embodiments, the Cas13 nuclease comprises an amino acid sequence having at least (e.g., greater than or equal to) about 60%, 65%, 70%, 75%, 80%, 85%, 86%, 87%, 88%, 89%, 90%, 91%, 92%, 93%, 94%, 95%, 96%, 97%, 98%, 99%, or 100% sequence similarity or identity to the amino acid sequence of a nuclease selected from the group consisting of Cas13a, Cas13b, 13bt1, Cas13bt2, Cas13c, Cas13d, RfxCas13d, and Cas13e. In some embodiments, the Cas13 nuclease comprises an amino acid sequence having at least (e.g., greater than or equal to) about 60%, 65%, 70%, 75%, 80%, 85%, 86%, 87%, 88%, 89%, 90%, 91%, 92%, 93%, 94%, 95%, 96%, 97%, 98%, 99%, or 100% sequence identity to SEQ ID NO: 1.

In some embodiments of the method, the modified T cell further comprises modified a CAR. In some embodiments, the CAR binds to an antigen expressed by a tumor. In some embodiments, the CAR binds to an antigen selected from the group consisting of Her-2, B7-H3, GPC2, GD2, CD19, CD20, CD22, MAGE, BAGE, CAGE, GAGE, HAGE, LAGE, PAGE, PRAME, NY-ESO-1, NY-SEO-1, tyrosinase, Melan-A/MART, gp100, TRP-1, TRP-2, CD30, EGFR, FAP, CD33, CD123, PD-L1, IGF1R, CD4, CSPG4, B7-H4, NKG2D, CS1, CD138, EpCAM, EBNA3C, GPA7, CD244, CA-125, ETA, CEA, CD52, MUC5AC, c-Met, EGFR, FAB, WT-1, PSMA, AFP, CEA, and CTAG1B.

### XII. Methods for Increasing Anti-Tumor Activity of T Cells

The disclosure further provided methods for increasing the anti-tumor activity of a T cell. In some embodiments, the method comprises contacting a tumor cell with a genetically modified T cell of the disclosure, wherein contacting the tumor cell with the genetically modified T cell increases the expression of anti-tumor cytokines or kills the tumor cell. In some embodiments, the modified T cell expresses or comprises (i) a class 2 type VI-D CRISPR effector having RNA-guided RNA endonuclease activity of the disclosure, (ii) a guide array of the disclosure, or (iii) both (i) and (ii). In some embodiments of the method, contacting the tumor cell with a genetically modified T cell of the disclosure increases the anti-tumor activity compared to a control T cell that does not comprise (i) or (ii) or both (i) and (ii).

In some embodiments, the method is an in vitro method, such that tumor cell is contacted with a genetically modified T cell of the disclosure in culture. In some embodiments, the method is an ex vivo method, wherein a T cell is isolated from a subject having cancer or a tumor, and the T cell is modified in culture to express a class 2 type VI-D CRISPR effector having RNA-guided RNA endonuclease activity of the disclosure and a guide array of the disclosure. After ex vivo modification, the modified T cell can be administered to the subject (e.g., an autologous treatment). In some embodiments, the method is an in vivo method, wherein an effective amount of a modified T cell of the disclosure is administered to the subject.

In some embodiments of the method, the modified T cell comprises (i) an expression vector comprising a nucleic acid sequence encoding a class 2 type VI-D CRISPR effector having RNA-guided RNA endonuclease activity; and (ii) an expression vector comprising a nucleic acid sequence encoding a guide array comprising one or more crRNA molecules, wherein the crRNA molecules independently comprise a direct repeat sequence and a spacer sequence that binds a target mRNA, and wherein the crRNA molecules bind to different target mRNAs or different regions of the same target mRNA

In some embodiments, the guide array comprises crRNA molecules that bind to an mRNA encoding a protein associated with T cell exhaustion. In some embodiments, the protein associated with T cell exhaustion is selected from the group consisting of FAS, ZC3H12A, CTLA4, SOCS1, CBLB, PD-1, TOX, TOX2, FLI1, CD5, CD39, CD46, TRAC, B2M, JUNB, IRF4, PDCD1 (PD-1), HAVCR2 (TIM3), DHX37, and combinations thereof.

In some embodiments, the class 2 type VI-D CRISPR effector having RNA-guided RNA endonuclease activity is a Cas13 nuclease selected from the group consisting of Cas13a, Cas13b, 13bt1, Cas13bt2, Cas13c, Cas13d, RfxCas13d, Cas13e, and functional variants thereof. In some embodiments, the Cas13 nuclease comprises an amino acid sequence having at least (e.g., greater than or equal to) about 60%, 65%, 70%, 75%, 80%, 85%, 86%, 87%, 88%, 89%, 90%, 91%, 92%, 93%, 94%, 95%, 96%, 97%, 98%, 99%, or 100% sequence similarity or identity to the amino acid sequence of a nuclease selected from the group consisting of Cas13a, Cas13b, 13bt1, Cas13bt2, Cas13c, Cas13d, RfxCas13d, and Cas13e. In some embodiments, the Cas13 nuclease comprises an amino acid sequence having at least (e.g., greater than or equal to) about 60%, 65%, 70%, 75%, 80%, 85%, 86%, 87%, 88%, 89%, 90%, 91%, 92%, 93%, 94%, 95%, 96%, 97%, 98%, 99%, or 100% sequence identity to SEQ ID NO: 1.

In some embodiments of the method, the modified T cell further comprises modified a CAR. In some embodiments, the CAR binds to an antigen expressed by a tumor. In some embodiments, the CAR binds to an antigen selected from the group consisting of Her-2, B7-H3, GPC2, GD2, CD19, CD20, CD22, MAGE, BAGE, CAGE, GAGE, HAGE, LAGE, PAGE, PRAME, NY-ESO-1, NY-SEO-1, tyrosinase, Melan-A/MART, gp100, TRP-1, TRP-2, CD30, EGFR, FAP, CD33, CD123, PD-L1, IGF1R, CD4, CSPG4, B7-H4, NKG2D, CSI, CD138, EpCAM, EBNA3C, GPA7, CD244, CA-125, ETA, CEA, CD52, MUC5AC, c-Met, EGFR, FAB, WT-1, PSMA, AFP, CEA, and CTAG1B.

In any of the embodiments described herein, the tumor can be selected from the group consisting of Acanthoma, Acinic cell carcinoma, Acoustic neuroma, Acral lentiginous melanoma, Acrospiroma, Acute eosinophilic leukemia, Acute lymphoblastic leukemia, Acute megakaryoblastic leukemia, Acute monocytic leukemia, Acute myeloblastic leukemia with maturation, Acute myeloid dendritic cell leukemia, Acute myeloid leukemia, Acute promyelocytic leukemia, Adamantinoma, Adenocarcinoma, Adenoid cystic carcinoma, Adenoma, Adenomatoid odontogenic tumor, Adrenocortical carcinoma, Adult T-cell leukemia, Aggressive NK-cell leukemia, AIDS-Related Cancers, AIDS-related lymphoma, Alveolar soft part sarcoma, Ameloblastic fibroma, Anal cancer, Anaplastic large cell lymphoma, Anaplastic thyroid cancer, Angioimmunoblastic T-cell lymphoma, Angiomyolipoma, Angiosarcoma, Appendix cancer, Astrocytoma, Atypical teratoid rhabdoid tumor, Basal cell carcinoma, Basal-like carcinoma, B-cell leukemia, B-cell lymphoma, Bellini duct carcinoma, Biliary tract cancer, Bladder cancer, Blastoma, Bone Cancer, Bone tumor, Brain Stem Glioma, Brain Tumor, Breast Cancer, Brenner tumor, Bronchial Tumor, Bronchioloalveolar carcinoma, Brown tumor, Burkitt's lymphoma, Cancer of Unknown Primary Site, Carcinoid Tumor, Carcinoma, Carcinoma in situ, Carcinoma of the penis, Carcinoma of Unknown Primary Site, Carcinosarcoma, Castleman's Disease, Central Nervous System Embryonal Tumor, Cerebellar Astrocytoma, Cerebral Astrocytoma, Cervical Cancer, Cholangiocarcinoma, Chondroma, Chondrosarcoma, Chordoma, Choriocarcinoma, Choroid plexus papilloma, Chronic Lymphocytic Leukemia, Chronic monocytic leukemia, Chronic myelogenous leukemia, Chronic Myeloproliferative Disorder, Chronic neutrophilic leukemia, Clear-cell tumor, Colon Cancer, Colorectal cancer, Craniopharyngioma, Cutaneous T-cell lymphoma, Degos disease, Dermatofibrosarcoma protuberans, Dermoid cyst, Desmoplastic small round cell tumor, Diffuse large B cell lymphoma, Dysembryoplastic neuroepithelial tumor, Embryonal carcinoma, Endodermal sinus tumor, Endometrial cancer, Endometrial Uterine Cancer, Endometrioid tumor, Enteropathy-associated T-cell lymphoma, Ependymoblastoma, Ependymoma, Epithelioid sarcoma, Erythroleukemia, Esophageal cancer, Esthesioneuroblastoma, Ewing Family of Tumor, Ewing Family Sarcoma, Ewing's sarcoma, Extracranial Germ Cell Tumor, Extragonadal Germ Cell Tumor, Extrahepatic Bile Duct Cancer, Extramammary Paget's disease, Fallopian tube cancer, Fetus in fetu, Fibroma, Fibrosarcoma, Follicular lymphoma, Follicular thyroid cancer, Gallbladder Cancer, Gallbladder cancer, Ganglioglioma, Ganglioneuroma, Gastric Cancer, Gastric lymphoma, Gastrointestinal cancer, Gastrointestinal Carcinoid Tumor, Gastrointestinal Stromal Tumor, Gastrointestinal stromal tumor, Germ cell tumor, Germinoma, Gestational choriocarcinoma, Gestational Trophoblastic Tumor, Giant cell tumor of bone, Glioblastoma multiforme, Glioma, Gliomatosis cerebri, Glomus tumor, Glucagonoma, Gonadoblastoma, Granulosa cell tumor, Hairy Cell Leukemia, Hairy cell leukemia, Head and Neck Cancer, Head and neck cancer, Heart cancer, Hemangioblastoma, Hemangiopericytoma, Hemangiosarcoma, Hematological malignancy, Hepatocellular carcinoma, Hepatosplenic T-cell lymphoma, Hereditary breast-ovarian cancer syndrome, Hodgkin Lymphoma, Hodgkin's lymphoma, Hypopharyngeal Cancer, Hypothalamic Glioma, Inflammatory breast cancer, Intraocular Melanoma, Islet cell carcinoma, Islet Cell Tumor, Juvenile myelomonocytic leukemia, Kaposi Sarcoma, Kaposi's sarcoma, Kidney Cancer, Klatskin tumor, Krukenberg tumor, Laryngeal Cancer, Laryngeal cancer, Lentigo maligna melanoma, Leukemia, Leukemia, Lip and Oral Cavity Cancer, Liposarcoma, Lung cancer, Luteoma, Lymphangioma, Lymphangiosarcoma, Lymphoepithelioma, Lymphoid leukemia, Lymphoma, Macroglobulinemia, Malignant Fibrous Histiocytoma, Malignant fibrous histiocytoma, Malignant Fibrous Histiocytoma of Bone, Malignant Glioma, Malignant Mesothelioma, Malignant peripheral nerve sheath tumor, Malignant rhabdoid tumor, Malignant triton tumor, MALT lymphoma, Mantle cell lymphoma, Mast cell leukemia, Mediastinal germ cell tumor, Mediastinal tumor, Medullary thyroid cancer, Medulloblastoma, Medulloblastoma, Medulloepithelioma, Melanoma, Melanoma, Meningioma, Merkel Cell Carcinoma, Mesothelioma, Mesothelioma, Metastatic Squamous Neck Cancer with Occult Primary, Metastatic urothelial carcinoma, Mixed Mullerian tumor, Monocytic leukemia, Mouth Cancer, Mucinous tumor, Multiple Endocrine Neoplasia Syndrome, Multiple Myeloma, Multiple myeloma, Mycosis Fungoides, Mycosis fungoides, Myelodysplastic Disease, Myelodysplastic Syndromes, Myeloid leukemia, Myeloid sarcoma, Myeloproliferative Disease, Myxoma, Nasal Cavity Cancer, Nasopharyngeal Cancer, Nasopharyngeal carcinoma, Neoplasm, Neurinoma, Neuroblastoma, Neuroblastoma, Neurofibroma, Neuroma, Nodular melanoma, Non-Hodgkin Lymphoma, Non-Hodgkin lymphoma, Nonmelanoma Skin Cancer, Non-Small Cell Lung Cancer, Ocular oncology, Oligoastrocytoma, Oligodendroglioma, Oncocytoma, Optic nerve sheath meningioma, Oral Cancer, Oral cancer, Oropharyngeal Cancer, Osteosarcoma, Osteosarcoma, Ovarian Cancer, Ovarian cancer, Ovarian Epithelial Cancer, Ovarian Germ Cell Tumor, Ovarian Low Malignant Potential Tumor, Paget's disease of the breast, Pancoast tumor, Pancreatic Cancer, Pancreatic cancer, Papillary thyroid cancer, Papillomatosis, Paraganglioma, Paranasal Sinus Cancer, Parathyroid Cancer, Penile Cancer, Perivascular epithelioid cell tumor, Pharyngeal Cancer, Pheochromocytoma, Pineal Parenchymal Tumor of Intermediate Differentiation, Pineoblastoma, Pituicytoma, Pituitary adenoma, Pituitary tumor, Plasma Cell Neoplasm, Pleuropulmonary blastoma, Polyembryoma, Precursor T-lymphoblastic lymphoma, Primary central nervous system lymphoma, Primary effusion lymphoma, Primary Hepatocellular Cancer, Primary Liver Cancer, Primary peritoneal cancer, Primitive neuroectodermal tumor, Prostate cancer, Pseudomyxoma peritonei, Rectal Cancer, Renal cell carcinoma, Respiratory Tract Carcinoma Involving the NUT Gene on Chromosome 15, Retinoblastoma, Rhabdomyoma, Rhabdomyosarcoma, Richter's transformation, Sacrococcygeal teratoma, Salivary Gland Cancer, Sarcoma, Schwannomatosis, Sebaceous gland carcinoma, Secondary neoplasm, Seminoma, Serous tumor, Sertoli-Leydig cell tumor, Sex cord-stromal tumor, Sezary Syndrome, Signet ring cell carcinoma, Skin Cancer, Small blue round cell tumor, Small cell carcinoma, Small Cell Lung Cancer, Small cell lymphoma, Small intestine cancer, Soft tissue sarcoma, Somatostatinoma, Soot wart, Spinal Cord Tumor, Spinal tumor, Splenic marginal zone lymphoma, Squamous cell carcinoma, Stomach cancer, Superficial spreading melanoma, Supratentorial Primitive Neuroectodermal Tumor, Surface epithelial-stromal tumor, Synovial sarcoma, T-cell acute lymphoblastic leukemia, T-cell large granular lymphocyte leukemia, T-cell leukemia, T-cell lymphoma, T-cell prolymphocytic leukemia, Teratoma, Terminal lymphatic cancer, Testicular cancer, Thecoma, Throat Cancer, Thymic Carcinoma, Thymoma, Thyroid cancer, Transitional Cell Cancer of Renal Pelvis and Ureter, Transitional cell carcinoma, Urachal cancer, Urethral cancer, Urogenital neoplasm, Uterine sarcoma, Uveal melanoma, Vaginal Cancer, Verner Morrison syndrome, Verrucous carcinoma, Visual Pathway Glioma, Vulvar Cancer, Waldenstrom's macroglobulinemia, Warthin's tumor, Wilms' tumor, and combinations thereof.

In any of the embodiments described herein, the CAR binds an antigen expressed by a tumor selected from the group consisting of Acanthoma, Acinic cell carcinoma, Acoustic neuroma, Acral lentiginous melanoma, Acrospiroma, Acute eosinophilic leukemia, Acute lymphoblastic leukemia, Acute megakaryoblastic leukemia, Acute monocytic leukemia, Acute myeloblastic leukemia with maturation, Acute myeloid dendritic cell leukemia, Acute myeloid leukemia, Acute promyelocytic leukemia, Adamantinoma, Adenocarcinoma, Adenoid cystic carcinoma, Adenoma, Adenomatoid odontogenic tumor, Adrenocortical carcinoma, Adult T-cell leukemia, Aggressive NK-cell leukemia, AIDS-Related Cancers, AIDS-related lymphoma, Alveolar soft part sarcoma, Ameloblastic fibroma, Anal cancer, Anaplastic large cell lymphoma, Anaplastic thyroid cancer, Angioimmunoblastic T-cell lymphoma, Angiomyolipoma, Angiosarcoma, Appendix cancer, Astrocytoma, Atypical teratoid rhabdoid tumor, Basal cell carcinoma, Basal-like carcinoma, B-cell leukemia, B-cell lymphoma, Bellini duct carcinoma, Biliary tract cancer, Bladder cancer, Blastoma, Bone Cancer, Bone tumor, Brain Stem Glioma, Brain Tumor, Breast Cancer, Brenner tumor, Bronchial Tumor, Bronchioloalveolar carcinoma, Brown tumor, Burkitt's lymphoma, Cancer of Unknown Primary Site, Carcinoid Tumor, Carcinoma, Carcinoma in situ, Carcinoma of the penis, Carcinoma of Unknown Primary Site, Carcinosarcoma, Castleman's Disease, Central Nervous System Embryonal Tumor, Cerebellar Astrocytoma, Cerebral Astrocytoma, Cervical Cancer, Cholangiocarcinoma, Chondroma, Chondrosarcoma, Chordoma, Choriocarcinoma, Choroid plexus papilloma, Chronic Lymphocytic Leukemia, Chronic monocytic leukemia, Chronic myelogenous leukemia, Chronic Myeloproliferative Disorder, Chronic neutrophilic leukemia, Clear-cell tumor, Colon Cancer, Colorectal cancer, Craniopharyngioma, Cutaneous T-cell lymphoma. Degos disease, Dermatofibrosarcoma protuberans, Dermoid cyst, Desmoplastic small round cell tumor, Diffuse large B cell lymphoma, Dysembryoplastic neuroepithelial tumor, Embryonal carcinoma, Endodermal sinus tumor, Endometrial cancer, Endometrial Uterine Cancer, Endometrioid tumor,,Enteropathy-associated T-cell lymphoma, Ependymoblastoma, Ependymoma, Epithelioid sarcoma, Erythroleukemia, Esophageal cancer, Esthesioneuroblastoma, Ewing Family of Tumor, Ewing Family Sarcoma, Ewing's sarcoma, Extracranial Germ Cell Tumor, Extragonadal Germ Cell Tumor, Extrahepatic Bile Duct Cancer, Extramammary Paget's disease, Fallopian tube cancer, Fetus in fetu, Fibroma, Fibrosarcoma, Follicular lymphoma, Follicular thyroid cancer, Gallbladder Cancer, Gallbladder cancer, Ganglioglioma, Ganglioneuroma, Gastric Cancer, Gastric lymphoma, Gastrointestinal cancer, Gastrointestinal Carcinoid Tumor, Gastrointestinal Stromal Tumor, Gastrointestinal stromal tumor, Germ cell tumor, Germinoma, Gestational choriocarcinoma, Gestational Trophoblastic Tumor, Giant cell tumor of bone, Glioblastoma multiforme, Glioma, Gliomatosis cerebri, Glomus tumor, Glucagonoma, Gonadoblastoma, Granulosa cell tumor, Hairy Cell Leukemia, Hairy cell leukemia, Head and Neck Cancer, Head and neck cancer, Heart cancer, Hemangioblastoma, Hemangiopericytoma, Hemangiosarcoma, Hematological malignancy, Hepatocellular carcinoma, Hepatosplenic T-cell lymphoma, Hereditary breast-ovarian cancer syndrome, Hodgkin Lymphoma, Hodgkin's lymphoma, Hypopharyngeal Cancer, Hypothalamic Glioma, Inflammatory breast cancer, Intraocular Melanoma, Islet cell carcinoma, Islet Cell Tumor, Juvenile myelomonocytic leukemia, Kaposi Sarcoma, Kaposi's sarcoma, Kidney Cancer, Klatskin tumor, Krukenberg tumor, Laryngeal Cancer, Laryngeal cancer, Lentigo maligna melanoma, Leukemia, Leukemia, Lip and Oral Cavity Cancer, Liposarcoma, Lung cancer, Luteoma, Lymphangioma, Lymphangiosarcoma, Lymphoepithelioma, Lymphoid leukemia, Lymphoma, Macroglobulinemia, Malignant Fibrous Histiocytoma, Malignant fibrous histiocytoma, Malignant Fibrous Histiocytoma of Bone, Malignant Glioma, Malignant Mesothelioma, Malignant peripheral nerve sheath tumor, Malignant rhabdoid tumor, Malignant triton tumor, MALT lymphoma, Mantle cell lymphoma, Mast cell leukemia, Mediastinal germ cell tumor, Mediastinal tumor, Medullary thyroid cancer, Medulloblastoma, Medulloblastoma, Medulloepithelioma, Melanoma, Melanoma, Meningioma, Merkel Cell Carcinoma, Mesothelioma, Mesothelioma, Metastatic Squamous Neck Cancer with Occult Primary, Metastatic urothelial carcinoma, Mixed Mullerian tumor, Monocytic leukemia, Mouth Cancer, Mucinous tumor, Multiple Endocrine Neoplasia Syndrome, Multiple Myeloma, Multiple myeloma, Mycosis Fungoides, Mycosis fungoides, Myelodysplastic Disease, Myelodysplastic Syndromes, Myeloid leukemia, Myeloid sarcoma, Myeloproliferative Disease, Myxoma, Nasal Cavity Cancer, Nasopharyngeal Cancer, Nasopharyngeal carcinoma, Neoplasm, Neurinoma, Neuroblastoma, Neuroblastoma, Neurofibroma, Neuroma, Nodular melanoma, Non-Hodgkin Lymphoma, Non-Hodgkin lymphoma, Nonmelanoma Skin Cancer, Non-Small Cell Lung Cancer, Ocular oncology, Oligoastrocytoma, Oligodendroglioma, Oncocytoma, Optic nerve sheath meningioma, Oral Cancer, Oral cancer, Oropharyngeal Cancer, Osteosarcoma, Osteosarcoma, Ovarian Cancer, Ovarian cancer, Ovarian Epithelial Cancer, Ovarian Germ Cell Tumor, Ovarian Low Malignant Potential Tumor, Paget's disease of the breast, Pancoast tumor, Pancreatic Cancer, Pancreatic cancer, Papillary thyroid cancer, Papillomatosis, Paraganglioma, Paranasal Sinus Cancer, Parathyroid Cancer, Penile Cancer, Perivascular epithelioid cell tumor, Pharyngeal Cancer, Pheochromocytoma, Pineal Parenchymal Tumor of Intermediate Differentiation, Pineoblastoma, Pituicytoma, Pituitary adenoma, Pituitary tumor, Plasma Cell Neoplasm, Pleuropulmonary blastoma, Polyembryoma, Precursor T-lymphoblastic lymphoma, Primary central nervous system lymphoma, Primary effusion lymphoma, Primary Hepatocellular Cancer, Primary Liver Cancer, Primary peritoneal cancer, Primitive neuroectodermal tumor, Prostate cancer, Pseudomyxoma peritonei, Rectal Cancer, Renal cell carcinoma, Respiratory Tract Carcinoma Involving the NUT Gene on Chromosome 15, Retinoblastoma, Rhabdomyoma, Rhabdomyosarcoma, Richter's transformation, Sacrococcygeal teratoma, Salivary Gland Cancer, Sarcoma, Schwannomatosis, Sebaceous gland carcinoma, Secondary neoplasm, Seminoma, Serous tumor, Sertoli-Leydig cell tumor, Sex cord-stromal tumor, Sezary Syndrome, Signet ring cell carcinoma, Skin Cancer, Small blue round cell tumor, Small cell carcinoma, Small Cell Lung Cancer, Small cell lymphoma, Small intestine cancer, Soft tissue sarcoma, Somatostatinoma, Soot wart, Spinal Cord Tumor, Spinal tumor, Splenic marginal zone lymphoma, Squamous cell carcinoma, Stomach cancer, Superficial spreading melanoma, Supratentorial Primitive Neuroectodermal Tumor, Surface epithelial-stromal tumor, Synovial sarcoma, T-cell acute lymphoblastic leukemia, T-cell large granular lymphocyte leukemia, T-cell leukemia, T-cell lymphoma, T-cell prolymphocytic leukemia, Teratoma, Terminal lymphatic cancer, Testicular cancer, Thecoma, Throat Cancer, Thymic Carcinoma, Thymoma, Thyroid cancer, Transitional Cell Cancer of Renal Pelvis and Ureter, Transitional cell carcinoma, Urachal cancer, Urethral cancer, Urogenital neoplasm, Uterine sarcoma, Uveal melanoma, Vaginal Cancer, Verner Morrison syndrome, Verrucous carcinoma, Visual Pathway Glioma, Vulvar Cancer, Waldenstrom's macroglobulinemia, Warthin's tumor, Wilms' tumor, and combinations thereof.

### Examples

The following Example describes a representative, non-limiting method of the disclosure.

### METHODS

### Cloning of Lentiviral Constructs

All oligonucleotides were synthesized by IDT or by the Stanford Protein and Nucleic Acid (PAN) Facility. To facilitate flexible cloning of whole guide arrays and avoid hairpin formation near the Esp31 digest site, pLentiRNAGuide_002 (Addgene #138151) was modified to remove the direct repeat region (pSLQ4419). Sequences for the high-affinity 14g2a-GD2 (E101K) chimeric antigen receptor (HA-28z CAR) and mCherry-P2A-Ruminococcus flavefaciens Cas13d (RfxCas13d) (Addgene #155305) sequences were previously described^{7,48}. The HA-28z CAR sequence was cloned into a pHR lentiviral backbone with a constitutive SFFV promoter (pSLQ5263). Regulatable RfxCas13d-DD was constructed by fusing the destabilizing domain (DD) from Escherichia coli dihydrofolate reductase (DHFR) to the C-terminus of RfxCas13d with a short glycine-serine linker.

RfxCas13d spacer sequences were generated using the previously-described cas13design web tool (see www.cas13design.nygenome.org) 50 and can be found in Tables 1 and 2. Guide array constructs were cloned using the following methods: For traditional restriction-ligation cloning, forward and reverse oligonucleotides for each spacer were annealed together, phosphorylated using T4 PNK (NEB), and ligated into backbone using T4 DNA ligase (NEB). For In-Fusion HD assembly (Takara), whole guide arrays were amplified using PCR primers containing 15-20 nt long 5′ and 3′ homology regions The PCR amplicon was then directly inserted into backbone. For NEBuilder HiFi assembly (NEB), single-stranded oligonucleotides with 20-30 nt long S′ and 3′ homology regions were synthesized and directly inserted into backbone.

Longer (5- and 10-plex) guide arrays were cloned using a two-step overlap extension PCR method. Arrays were synthesized as single-stranded oligo fragments encoding a direct repeat flanked by two overlapping spacer regions (see Figure 5A and Table 3). In the first PCR reaction, oligo pairs were annealed and extended. In the second PCR reaction, double-stranded fragments were pooled together and amplified without primers for 15 cycles. Then, forward and reverse primers containing 5′ and 3′ homology regions were spiked in before continuing for another 15-20 cycles. PCR amplicons were run on a 2% agarose gel, purified, and inserted into backbone using NEBuilder HiFi assembly.

### Cell Culture

Lenti-X 293T cells (Takara) were cultured in 0.22 μm sterile-filtered DMEM (Gibco) supplemented with 10% fetal bovine serum (FBS). Nalm6-GD2 cells were cultured in 0.22 μm sterile-filtered complete medium (CM) [RPMI supplemented with 10% FBS, 100 U ml⁻¹ penicillin, and 100 μg ml⁻¹ streptomycin (Gibco)]. All cells were cultured in humidified incubators at 37° C. and 5% CO₂.

### Primary Human T Cell Isolation and Expansion

Buffy coats derived from anonymous healthy blood donors were purchased from the Stanford Blood Center. Primary human T cells were isolated using the EasySep Human T cell Isolation kit (STEMCELL) according to the manufacturer's protocol with Ficoll-Paque PLUS (GE Healthcare) and SepMate-50 tubes. Isolated T cells were immediately cryopreserved at 4-10×10⁶ cells per vial in either FBS supplemented with 10% DMSO (Sigma) or CryoStor (STEMCELL).

Cryopreserved T cells were thawed and activated same-day using Human T-Activator CD3/CD28 Dynabeads (Gibco) at a 3:1 bead-to-cell ratio. T cells were cultured in human T cell medium (HTCM) [0.22 μm sterile-filtered AIM V supplemented with 5% FBS, 10 mM HEPES, 2 mM GlutaMAX, 100 U ml⁻¹ penicillin, and 100 μg ml⁻¹ streptomycin (Gibco)]. Human recombinant IL-2 (STEMCELL) was provided at 100 U ml⁻¹. On day 3 of culture, Dynabeads were magnetically removed. Cells were expanded every other day by adding HTCM to maintain an overall cell concentration of 0.5-1×10⁶ cells per ml. Live cell counts were obtained following the manufacturer's protocol for Trypan Blue exclusion using the Countess 3 automated cell counter (Invitrogen).

For culture conditions requiring trimethoprim (TMP), a 1000 μM concentrated stock solution of TMP (in DMSO) was freshly thawed and added to cells starting on day 3 of culture to a final concentration of 1 μM unless otherwise noted. To maintain culture conditions, cells were expanded every other day in HTCM supplemented with 1 μM TMP unless otherwise noted. To remove TMP, cells were washed twice with FACS buffer [DPBS supplemented with 2% FBS and 1 mM EDTA (Gibco)] and resuspended in fresh prewarmed HTCM.

### Lentiviral Preparation

7.5×10⁵ Lenti-X cells were seeded in 6-well plates overnight containing 2 ml DMEM supplemented with 10% FBS. The next morning, 850 μl culture medium was removed and cells were transfected with 0.55 μg pMD2.G (Addgene plasmid #12259), 1.28 μg psPAX2 (Addgene plasmid #12260), and 1.79 μg transfer plasmid in 426 μl Opti-MEM (Gibco) using 10 88 μl TransIT-LTI (Mirus Bio). Six hours later, culture medium was completely removed and replaced with fresh DMEM supplemented with 10% FBS and 1× ViralBoost (Alstem Bio). 24 hours after transfection, viral supernatant was harvested and filtered through a 0.45 μm syringe filter (MilliporeSigma). The supernatant was mixed with lentivirus precipitation solution (Alstem Bio), incubated at 4° C. for a minimum of 4 hours, and concentrated 10-100× in Opti-MEM following the manufacturer's protocol. Concentrated virus was either used fresh or kept frozen at −80° C. for future use. Lentiviral production in 10- and 15-cm dishes were scaled up proportionally to culture vessel surface area.

### Immunostaining and Flow Cytometry

mCherry fluorescence was used as a quantitative measure of RfxCas13d expression in all flow cytometry experiments. The 1A7 anti-14G2a idiotype antibody used to detect HA-28z CAR surface expression was conjugated in-house with the DyLight 650 antibody labelling kit (Thermo Fisher). The following fluorescent antibodies were used to stain human T cell surface markers: [TIM3-BV421 (clone F38-2E2), LAG3-FITC (clone 11C3C65), PD-1-APC (clone A17188B), FAS-FITC (clone DX2), CTLA4-APC (clone BNI3), CD46-APC (clone TRA-2-10), CD62L-PerCP-Cy5.5 (clone DREG-56), and CD45RA-PE-Cy7 (clone HI100)] (BioLegend), [CD8-AF405 (clone 3B5)] (Invitrogen). Approximately 1-2× 10⁵ cells were resuspended in 100 μl FACS buffer and stained at room temperature for 20 minutes. Samples probing for CAR surface expression were stained on ice for 10 minutes to minimize receptor internalization. Cells were washed twice with FACS buffer prior to flow cytometry, which was conducted on a CytoFLEX S (Beckman Coulter). For all samples, a minimum of approximately 1 ×10⁴ events was collected within the final gated population-of-interest.

### Transduction of Primary Human T Cells

Primary human T cells were transduced with concentrated lentivirus at 1-10% v/v (unless otherwise noted) 24-48 hours after bead activation. Construct expression was verified on day 5 of culture via flow cytometry as described above. For constructs requiring puromycin selection, a concentrated stock solution of 10 μg μl⁻¹ puromycin was thawed and added to cells on day 3 of culture (24-48 hours after transduction) to a final concentration of 1 μg ml⁻¹. Puromycin selection was complete by day 5 of culture.

### Cell Sorting

On day 5 of culture, primary human T cells were spun down and resuspended in ice-cold FACS buffer at a concentration of 1-2× 10⁷ cells per ml. Cells were sorted on a Sony SH800 sorter (Sony Biotechnology) using a 130 μm chip at low sample pressure on “semi-purity” or “purity” mode. Mock untransduced T cells were used to determine RfxCas13d-gating. Live RfxCas13d+ cells were sorted into collection tubes containing cold HTCM. After sorting, cells were spun down and resuspended in fresh prewarmed HTCM supplemented with 100 U ml⁻¹ IL-2 to a final concentration of approximately 3-5×10⁵ cells per ml. After sorting, cells were expanded as described above.

### RNA Extraction and Quantitative Reverse Transcription PCR (RT-qPCR)

Total RNA was extracted from approximately 1-10×10⁶ primary human T cells using the RNEasy Plus kit (QIAGEN) according to the manufacturer's protocol using additional 2-mercaptoethanol. RNA concentration was measured using a NanoDrop One (Thermo Fisher) and normalized across all cell samples prior to reverse transcription using the iScript cDNA Synthesis kit (Bio-Rad). Diluted cDNA was added to iTaq Universal SYBR Green Supermix (Bio-Rad) and run on a CFX384 Touch (Bio-Rad) with the following PCR conditions: 50° C. for 10 min, 95° C. for 30 s, 40×(95° C. for 10 s, 60° C. for 30 s). Gene expression was quantified using the 2-44Ct method using SDHA as an internal control. Primer sequences can be found in Table 3.

### Bulk RNA-Seq and Differential Expression Analysis

Total RNA was extracted from approximately 5×10⁶ primary human T cells on day 10 of culture, as described above. Bulk RNA-seq was performed by Novogene (Davis, CA) on an Illumina NovaSeq 6000 with 20-30×10⁶150-bp paired-end reads per sample. Transcript abundance was quantified from raw reads using kallisto⁸¹. Custom scripts in R (version 4.1.2) were used to perform differential expression analysis: quants were imported using tximeta⁸² and batch-corrected and analyzed using DESeq2⁸³, and resulting log 2 fold-changes were shrunken using apeglm⁸⁴ and visualized using ggplot2.

### Design and Assembly of Pooled Double Guide Array Library

A custom, curated library of 24 putative exhaustion-related genes was designed based on results from prior CRISPR-Cas9 proliferation and functional knockout screens in primary human T cells (see Table 3). Three top-ranking 23-nt guide spacer sequences targeting the CDS of the broadest range of human transcript isoforms were generated per gene as described above using the cas13design tool⁵⁰, yielding 72 targeting guide spacers. In addition, eight 23-nt random non-targeting guide spacers that did not align to the human transcriptome (as confirmed by nucleotide BLAST) were generated. In total, 80 guide spacers were generated. A custom Python script was used to concatenate RfxCas13d direct repeats to spacer sequences and to output all pairwise guide array combinations, resulting in 80×80=6,400 double guide array sequences (see Table 3). Flanking S′ and 3′ homology regions were added to facilitate insertion into the pSLQ4419 backbone as described above. The guide array library was synthesized as a single-stranded oligo pool (Twist Bioscience) and 10 ng template was amplified using KAPA HiFi HotStart (Roche) with the following PCR conditions: 95° C. for 3 min, 8×(98° C. for 20 s, 55° C. for 15 s, 72° C. for 15 s), 72° C. for 1 min. The reaction product was recovered using a NucleoSpin PCR Clean-up kit (Macherey-Nagel) and run on a 2100 Bioanalyzer (Agilent) by the Stanford PAN Facility for sizing and quantification (85% double guide arrays) and quality control (minimal PCR overamplification). The amplified library was efficiently cloned, with less than 5% background, into pSLQ4419 (Esp31) using NEBuilder HiFi DNA assembly and variable spacer regions matching the nucleotide distribution of the library were confirmed by Sanger sequencing. Complete library representation with minimal bias⁸⁵ (Gini coefficient: 0.146, 90^{th} percentile/10th percentile of guide array reads: 1.96, see FIG. 10A) was further verified by Miseq v3 600-cycle paired-end sequencing (Illumina).

### CD8⁺CAR T Cell Proliferation Screen

8×10⁷ primary human T cells (adjusted 1:1 CD4⁺-to-CD8⁺ ratio) were thawed and activated 3:1 with CD3/CD28 Dynabeads same-day, as described above. Throughout the screen, CD8⁺ T cells were cultured together with CD4+ T cells in HTCM supplemented with 100 U ml⁻¹ IL-2. On day 1 of culture, cells were co-transduced with the following freshly harvested and 100× concentrated lentiviruses (prepared as described above with endotoxin-free plasmid DNA): 2% v/v mCherry-P2A-RfxCas13d, 1.5% v/v HA-28z CAR, and 0.2% v/v pooled double guide array library. On day 3 of culture, cells were selected with 1 μg ml-puromycin over 48 hours. Approximately 28.9% of cells survived puromycin selection, corresponding to ˜83.9% single lentiviral integrations (assuming independent transduction events following the Poisson distribution). On day 5, a portion of cells was stained and flowed for HA-28z CAR and CD8 expression to determine the overall percentage of CAR⁺CD8⁺ T cells. This percentage informed the subsequent FACS of RfxCas13d+ cells and ensured that a sufficient number of cells was sorted to achieve library coverage of approximately 1000× within the CAR⁺CD8⁺ T cell subpopulation. Post-sort, cells were split into two replicates at ˜1000⁺ representation and expanded every other day as described above. On days 11, 13, and 15 of culture, a portion of cells was stained and flowed to determine the percentage of CD8⁺, RfxCas13d″, and CAR⁺ cells. Following flow cytometry, CD8⁺ T cells were magnetically isolated from the total T cell culture and collected at ˜1000⁺ representation using the EasySep human CD8 Positive Selection kit (STEMCELL). The proliferation screen was terminated after 15 days of ex vivo expansion.

### Genomic DNA Extraction and Library Preparation

Genomic DNA (gDNA) was extracted from magnetically isolated CD8⁺ T cells using the DNeasy Blood and Tissue kit (QIAGEN). Cells were lysed using Buffer AL and spun down at maximum speed for three minutes to pellet magnetic positive selection beads. The cell lysate supernatant was then carefully transferred into a clean tube before proceeding with the manufacturer's protocol for gDNA isolation.

The double guide array library was prepped for next-generation sequencing through a two-step PCR protocol. Briefly, PCR 1 primers targeted and amplified lentivirally-integrated guide array sequences, appended Illumina sequencing primer binding sites, and increased the overall sequence diversity of the amplicon with eight custom stagger sequences to improve cluster identification. PCR2 primers appended paired-end indexes for sample identification and contained Illumina P5/P7 sequences that bound to the flow cell. Primer sequences for both PCR1 and PCR2 can be found in Table 3. For each collected sample, a total of ˜40 μg gDNA was PCR1 amplified (6 μg gDNA roughly corresponds to 1×10⁶ cells) as follows: a maximum of 2 μg gDNA template was amplified per 50 μl reaction with the following cycling conditions: 95° C. for 3 min, 24×(98° C. for 20 sec, 70° C. for 15 sec, 72° C. for 15 sec), 72° C. for 1 min. 200 ng of plasmid DNA (pDNA) library was also PCR1 amplified. The PCR1 products were pooled together by sample and column purified. Approximately 10 ng of each purified PCR1 product was loaded as template DNA for PCR2 with the following cycling conditions: 95° C. for 3 min, 12×(98° C. for 20 sec, 65° C. for 15 sec, 72° C. for 15 sec), 72° C. for 1 min. All reactions were set up with KAPA HiFi HotStart (Roche). PCR2 products were run on a 2% ultra-pure agarose gel (Invitrogen), bands were gel-extracted using the Nucleospin Gel Clean-up kit (Macherey-Nagel), and DNA was quantified on a Qubit fluorometer (Invitrogen).

### Screen Readout and Analysis

CRISPR guide array enrichment was read out via pooled Miseq v3 600-cycle paired-end sequencing (Illumina). Samples were accurately identified according to paired-end indexes. Raw fastq files were 5′ and 3′ adapter-trimmed and filtered by quality (−q 30) and length using cutadapt. Processed reads were aligned to a custom index built from a reference library of all double guide array combinations using bowtie2 end-to-end alignment⁸⁶. Guide array counts were generated from alignment data using samtools.

Screening data was analyzed using the DESeq2 negative binomial distribution model within a custom R script. Technical replicate pair correlation was evaluated by linear regression of normalized replicate count data. Significant guide array enrichment and depletion were analyzed via pairwise comparisons using the Wald test to calculate log 2 fold-changes and adjusted p values for each guide array. Based on previously-described methods⁸⁷, log 2 fold-changes were determined by comparing array abundance in collected cell samples (collected on days 11, 13, or 15 of culture) to array abundance in the original plasmid DNA prep as a starting timepoint. The likelihood ratio test was executed by comparing full (˜timepoint) and reduced (˜1) models. These results were used to rank guide arrays based on how significantly attributed their count variation across samples was to the (reduced) timepoint variable. Significant gene-level enrichment and depletion were determined using robust rank aggregation⁸⁸ of guide-level Wald test results. Data was visualized using ggplot2 and gene-level hierarchical clustering was performed using pheatmap.

### Cytokine Secretion

On day 10 of culture, primary human T cells and GFP⁺Nalm6-GD2 cells were spun down and resuspended in fresh prewarmed CM. 1×10⁵ GFP⁺Nalm6-GD2 cells were seeded in clear flat-bottom 96-well plates and co-cultured with 1×10⁵ primary human HA-28z CAR T cells in a total volume of 200 μl CM. Triplicate wells were plated per condition. After 24 hours, the plate was spun down and the co-culture supernatant was collected for immediate analysis or frozen at −80° C. Cytokine concentration was measured using IFNγ or IL-2 ELISA Max kits (Biolegend) following the manufacturer's protocol.

### Tumor Killing and Serial Restimulation

In vitro tumor killing and serial restimulation assays were performed on an IncuCyte S3 (Sartorius). On day 10 of culture, primary human T cells and GFP⁺Nalm6-GD2 cells were spun down and resuspended in fresh prewarmed CM. 5×10⁴ GFP⁺Nalm6-GD2 cells were seeded in clear flat-bottom 96-well plates and co-cultured with primary human HA-28z CAR T cells at varying effector: target (E: T) ratios as indicated in figure legends in a total volume of 200 μl CM. Triplicate wells were plated per condition, and four images were acquired per well over a total of 48-72 hours. Total integrated GFP intensity per well was used as a metric to quantify live GFP⁺Nalm6-GD2 cells. All values were normalized to the corresponding initial scan and plotted over time using the IncuCyte analysis software. For serial restimulation experiments, GFP⁺Nalm6-GD2 and HA-28z CAR T cells were co-cultured as described above. After 48-72 hours of initial stimulation, the remaining cells were spun down, resuspended in fresh prewarmed CM, counted, and co-cultured with an additional 5×10⁴ GFP⁺Nalm6-GD2 at varying E: T ratios as described above. This process was repeated twice for a total of three stimulations.

### Detection of Metabolites in Cell Culture Media

On day 10 of culture, 5×10⁴ primary human HA-28z CAR T cells were seeded in flat-bottom 96-well plates in a total volume of 200 μl AIM-V without phenol red (Gibco) supplemented with 100 U ml⁻¹ IL-2. ATP (Sigma) was spiked-in to a final concentration of 20 μM. Cells were incubated for one hour (unless otherwise noted) at 37° C. and 5% CO₂. Cells were then spun down and culture supernatant was collected for immediate analysis using ATP (Promega), AMP (Promega), and adenosine (Abcam) detection kits following the manufacturer's protocol.

### Data Representation

Box and violin plots were created using ggplot2 and represent at least ˜1×10⁴ cells per sample. Box plots indicate the median as well as first and third quartiles. Whiskers extend from the box to no more than 1.5⁺ the distance between first and third quartiles. Unless otherwise noted, all bar graphs show the mean and standard deviation of replicates, which are plotted as points.

### Statistical Analysis

Unless otherwise noted, statistical analyses for significance between groups were conducted using either ordinary or repeated measures one-way ANOVA with correction for multiple comparisons testing (using Dunnett's test) in GraphPad Prism 9. Linear regression analyses were conducted in R or in GraphPad Prism 9.

### RESULTS

### Development of Cas13d for Optimal Expression and Activity in Primary Human T Cells

The CRISPR/Cas13d system derived from Ruminococcus flavefaciens XPD3002 (RfxCas13d) exhibits superior catalytic activity and targeting specificity in mammalian cells^{42,48}. We initially confirmed the functional activity of lentivirally-integrated RfxCas13d in primary human T cells by characterizing crRNA-guided knockdown of a transgenic GFP reporter (FIGS. 8A-B). We then sought to target endogenous genes relevant to T cell exhaustion in HA-28z CAR T cells. Antigen-independent clustering of the HA-28z CAR results in tonic signaling and exhaustion-associated reprogramming, including the upregulation of inhibitory receptors LAG3, PD-1, and TIM3, by day 10 of ex vivo culture⁷. Prior studies have demonstrated that blocking these canonical exhaustion markers is of clinical significance⁴⁹. We therefore hypothesized that MEGA HA-28z CAR T cells, which co-express RfxCas13d and a targeting guide RNA array, could simultaneously suppress the upregulation of LAG3, PD-1, and TIM3 (FIG. 1A). We first designed three single guides per gene⁵⁰ and evaluated knockdown efficiency by measuring the corresponding surface expression of LAG3/PD-1/TIM3 by flow cytometry on day 10, in comparison to a non-targeting control guide (FIGS. 8C-D). Using a bicistronic CRISPR system, we observed limited knockdown across three different donors due to inefficient lentiviral transduction of RfxCas13d (FIG. 8E). Interestingly, we discovered that all bicistronic configurations resulted in low functional viral titer due to either RfxCas13d array processing (in the forward orientation) or crRNA-guided cleavage (in the reverse orientation) of lentiviral RNA during packaging (FIGS. 8F-G). Guided by these data, we established an optimized workflow to manufacture MEGA HA-28z CAR T cells, in which primary human T cells were co-transduced with separate RfxCas13d and HA-28z CAR constructs prior to transduction with crRNA constructs (FIG. 1B, FIGS. 8H-I).

### MEGA CAR T Cells Robustly and Specifically Suppress Upregulation of Canonical Exhaustion Markers Driven by Tonic Signaling

To assess whether optimized MEGA HA-28z CAR T cells could suppress upregulation of LAG3, PD-1, and TIM3, we isolated primary T cells from multiple healthy blood donors and co-transduced cells with RfxCas13d and HA-28z CAR constructs. One day later, we transduced cells with either 1) single guides targeting each receptor, 2) double guide arrays targeting all pairwise combinations of receptors, or 3) a non-targeting control guide. This sequential transduction process ensured that RfxCas13d and CAR expression were uniform across all experimental conditions. We then sorted RfxCas13d+ cells using co-expressed mCherry as a marker on day 5 and measured receptor surface expression on day 10 using flow cytometry (FIG. 1B). As reported previously, we observed strong upregulation of LAG3/PD-1/TIM3 in HA-28z CAR T cells expressing a non-targeting guide in comparison to mock untransduced T cells (FIG. 1C). Single guides and double guide arrays specifically suppressed upregulation of each targeted receptor to near-baseline levels. These results were consistent across independent experiments with multiple donors (FIG. 1D).

To verify that inhibitory receptor suppression at the protein level was due to RfxCas13d activity at the transcript level, we isolated bulk RNA from MEGA HA-28z CAR T cells on day 10 and quantified the abundance of LAG3, PDCD1 (PD-1), and HAVCR2 (TIM3) mRNA transcripts relative to a non-targeting control using quantitative reverse transcription PCR (RT-qPCR) (FIG. 1E, FIG. 9A). We observed a strong correlation between transcript abundance and surface protein expression across all samples. Interestingly, we noted spacer-dependent positional effects within double guide arrays. While the LAG3 targeting spacer exhibited efficient knockdown regardless of array position, PD-1 and TIM3 targeting spacers were generally more effective in the 3′-proximal position. The knockdown efficiency of LAG3 and TIM3 spacers was also more robust across donors than the PD-1 spacer, which could be attributed to the narrower and more variable dynamic range of PD-1 upregulation across donors, or to a suboptimal PD-1 spacer sequence.

To evaluate potential transcriptome-wide off-target and/or collateral cleavage effects, we performed bulk RNA-Seq on RNA extracted from day 10 MEGA HA-28z CAR T cells generated from two donors. Recent studies have disputed whether collateral RfxCas 13d activity is correlated with on-target transcript cleavage activity in mammalian cells⁵¹⁻⁵³. We therefore chose to analyze LAG3, TIM3, and LAG3+ TIM3 targeting guides because they exhibited the greatest knockdown efficiency across donors. We identified significant on-target knockdown of LAG3 and/or HAVCR2 transcripts compared to a non-targeting control in all samples, with no significant (adjusted p value <0.001) off-target activity detected (FIG. 1F). We did not observe activation of interferon pathways in response to either crRNA expression or target RNA cleavage⁵⁴. Notably, we also did not observe off-target protein-level effects, as CAR surface expression was uniform across all targeting and non-targeting samples (FIG. 9B).

We next built triple guide arrays encoding all unique permutations of LAG3/PD-I/TIM3 targeting spacers to knock down all three inhibitory receptors simultaneously in MEGA HA-28z CAR T cells. All triple guide arrays were able to suppress upregulation of exhaustion markers across 2-3 different donors as measured by flow cytometry (FIG. 1G, FIG. 9C) Transcript abundance was strongly correlated with surface protein expression (FIG. 1H, FIG. 9D) and CAR expression was uniform across all samples as expected (FIG. 9E). Certain spacer configurations exhibited more robust triple knockdown efficiency than others, and this pattern was consistent with positional dependencies that were observed in the double guide array experiments. To check whether triple knockdown was occurring at the single cell level, we gated day 10 flow cytometry data for all combinations of LAG3^{+/-}PD-1^{+/-} TIM3^{+/-}cells (FIG. 1I, FIGS. 9F-G). We observed that the triple-positive population dropped from 35.01% in the non-targeting control to 5.46% in cells expressing the optimal triple guide array (LPT), with a concomitant increase in the triple-negative population from 15.80% to 55.09%. This indicates that MEGA can achieve highly multiplexed gene knockdown in primary human T cells.

### MEGA Facilitates Two-Dimensional Genetic Screening and Identifies Novel Paired Regulators of Proliferation in a Model of CD8⁺CAR T Cell Dysfunction

While CRISPR/Cas9 screens have identified individual genes that restrain T cell anti-tumor activity, little is known about the complex network of genetic interactions and broader gene programs that enforce T cell dysfunction and exhaustion. Combinatorial CRISPR screens, which perturb multiple genes per cell, offer a powerful method to decode these interactions, though no such studies have been performed before in primary human T cells^{55,56.}

The programmable targeting specificity of the MEGA platform, when coupled with efficient multiplexing, is highly amenable to combinatorial CRISPR screening, and the compact guide array allows easy deconvolution of perturbation information in each cell. We hypothesized that co-expression of RfxCas13d and a pooled guide array library would enable us to conduct systematic pairwise genetic perturbations in primary human T cells. We applied this technology in a proof-of-concept study to identify novel combinations of putative exhaustion-related genes that regulate the proliferation of dysfunctional CD8⁺ HA-28z CAR T cells in culture (FIG. 2A). We initially curated a list of 24 genes based on prior CRISPR-Cas9 knockout studies in primary human CD8⁺ T cells (see Table 3). We then designed and assembled a custom library of 5,184 double guide arrays targeting all 576 pairwise combinations of these genes (FIG. 10A, Methods, Table 3). Each targeting guide was also paired with a set of 8 randomly generated non-targeting guides to simulate an additional 1,152 “single” guide perturbations. Finally, as a control we included 64 paired non-targeting guides for a total of 6,400 unique guide arrays. We utilized a heuristic screening approach such that prior functional validation of targeting guides was unnecessary.

Briefly, we transduced T cells isolated from a single healthy donor with RfxCas13d, HA-28z CAR, and the pooled double guide array library (FIG. 2A, FIGS. 10B, see Methods). Cells were sorted for RfxCas13d expression on day 5 and split into two replicates to account for technical variation in culturing conditions and downstream processing. Cells were cultured in parallel over two weeks, with HA-28z tonic signaling driving both T cell expansion and subsequent dysfunction. CD8⁺ T cells were then magnetically isolated from the bulk population upon sample collection on days 11, 13, and 15 of culture. We quantified guide array abundance through PCR amplification and deep sequencing of the guide array cassette. Unlike multiplex Cas9 screens performed in cancer cell lines, which require multiple reads and complex barcoding schema to accurately identify guide pairs⁵⁵, our approach easily identified pairs without barcoding by directly sequencing the compact RfxCas13d guide array in a single read. Complete library coverage was supported by strong replicate correlation and non-zero counts for all guide arrays across all samples (FIGS. 10C-D).

We performed pairwise statistical testing to determine whether guide arrays were significantly enriched (targeting paired negative regulators of proliferation) or depleted (targeting gene pairings essential for proliferation) between early and late screening timepoints (FIGS. 2B-C). We identified numerous guide arrays that were consistently positively or negatively selected in proliferating HA-28z CAR T cells across replicates. As expected, non-targeting control arrays were neither enriched nor depleted. Single perturbations (those paired with a non-targeting spacer) generally resulted in a weaker effect on proliferation than double perturbations, apart from guides targeting IRF4, JUNB, and CBLB, which had observable dominant negative effects. We were initially surprised by the robust depletion of these guides due to the design of our library. However, prior studies suggest that despite their established roles in driving T cell dysfunction, the transcription factors IRF4 and JUNB are also critical for the expansion and persistence of T cells7, 31, 35, 57. The function of genes such as CBLB (negative regulator of T cell signalling¹⁴) in the context of CAR T dysfunction warrants further investigation-we note that the CBLB-1 spacer appears to have a dominant negative effect, while CBLB-2 and CBLB-3 spacers were enriched in certain guide combinations. Highly enriched double arrays frequently included FAS (such as FAS in combination with either ZC3H12A, CTLA4, or SOCS1), which is supported by prior work implicating FAS as a negative regulator of CAR T cell activity via activation-induced cell death^{58,59}. We also observed guide array positional effects that were consistent with our previous results, enabling us to sample from a larger perturbation space in which the relative level of repression between two target genes could be varied. We compiled guide array data and observed comparable results across broader gene-level interactions (FIGS. 9E-F). Lastly, we ranked guide arrays with the most significant count variation across all screening timepoints as determined by likelihood-ratio test and identified top hits consistent with pairwise analyses (FIG. 2D).

We chose a set of top-ranking guide array hits to functionally validate based on both guide- and gene-level data (FIG. 2E). We generated MEGA HA-28z CAR T cells from 3-4 different blood donors and individually perturbed either one of 7 significantly enriched gene pairs or one of 2 significantly depleted gene pairs. We tracked T cell expansion in culture until day 15 and computed normalized CD8⁺ fold-change expansion across all donors in comparison to a non-targeting control (FIG. 2F, FIG. 11A). Paired perturbations regulated T cell expansion in culture and recapitulated screening results in both CD8⁺ and bulk T cells (FIG. 2F, FIGS. 11A-C). Accordingly, we also noted a significantly smaller RfxCas13d+ population in bulk T cells expressing depleted arrays by day 5 (despite uniform CAR expression), suggesting that the dysregulation of proliferation is rapid and occurs within 48 hours post-transduction (FIGS. 11D-E). To explore whether proliferative differences could be explained by altered T cell differentiation, we measured surface CD62L/CD45RA expression on day 10 (FIGS. 11F-G). Effector memory phenotypes were more abundant in CAR T cells expressing enriched guide arrays (⁴¹²% when targeting CBLB+ FAS), while depleted guide arrays restrained effector differentiation and skewed cells toward central memory (^{426.4}% when targeting TOX2+JUNB) and stem cell memory (^{419.3}% when targeting PDCD1+IRF4) phenotypes.

### Paired Transcriptomic Perturbations Enhance the Anti-Tumor Activity of Dysfunctional CAR T Cells

Encouraged by our validation experiments, we next investigated whether top paired screening hits could improve not only the proliferative potential but also the anti-tumor activity of dysfunctional CAR T cells We performed a small-scale secondary screen of top enriched guide arrays using in vitro cytokine secretion and cytotoxicity in response to antigen-positive tumor as functional readouts (FIG. 3A). On day 10 of culture, we incubated MEGA HA-28z CAR T cells with Nalm6 leukemia cells expressing GD2 (Nalm6-GD2) and measured levels of IFN-γ and IL-2 in co-culture supernatant after 24 hours (FIGS. 3B-D, FIGS. 12A-B). Top guide arrays robustly augmented IFN-γ secretion in tonically signaling CAR T cells with and without antigen stimulation while IL-2 secretion was moderately improved upon antigen stimulation. To assess MEGA CAR T cell cytotoxicity, we monitored Nalm6-GD2 abundance within co-cultures over a period of 48-72 hours using live cell imaging. Top guide arrays markedly improved the tumor killing ability of dysfunctional CAR T cells in comparison to a non-targeting control across multiple donors and effector: target ratios (FIGS. 3E-F, FIG. 12C). This was especially apparent upon repeated antigen stimulation in a serial rechallenge assay-paired knockdown of CBLB and FAS potentiated a robust and durable anti-tumor response by exhausted CAR T cells when compared to a non-targeting control, which failed to control tumor outgrowth (FIGS. 3E-G). We hypothesize that dual targeting of CBLB and FAS improves CAR T cell activity through a potentially synergistic disruption of two orthogonal pathways that are known to negatively regulate T cell activation^{14,59,60} (FIG. 3H).

### MEGA Enables Rapid, Tunable, and Reversible Perturbation of the T Cell Transcriptome

In earlier experiments, we noted that transcript knockdown in cells expressing targeting crRNAs was wholly dependent on RfxCas13d expression (FIG. 13A). Based on this data, we were interested in whether modulating levels of RfxCas13d could enable tunable and reversible control of the T cell transcriptome. To complement the fast timescale at which RfxCas13d-mediated RNA degradation occurs, we fused a destabilization domain (DD) from the Escherichia coli dihydrofolate reductase to the C terminus of RfxCas13d (FIG. 4A). At steady-state, regulatable RfxCas13d-DD is rapidly degraded by the proteasome due to the disordered DD61. In the presence of trimethoprim (TMP), an FDA-approved small molecule antibiotic, tertiary structure of the DD is stabilized, allowing RfxCas13d-DD to bind to and target RNA.

We initially confirmed that RfxCas13d-DD protein expression was regulated by the presence of TMP in primary human T cells by staining for intracellular FLAG-tagged RfxCas13d (FIG. 13B). To characterize the activity and dynamic range of RfxCas 13d-DD for endogenous gene repression, we generated MEGA T cells expressing RfxCas13d-DD and either a CD46 (ubiquitous surface protein) targeting guide or a non-targeting guide. Following co-transduction, we cultured MEGA T cells in the presence or absence of TMP for 48 hours and measured CD46 surface expression on day 5 MEGA T cells conditionally repressed CD46 in a TMP-dependent manner, retained full functional activity in the presence of drug, exhibited minimal leaky activity in the absence of drug, and did not alter guide targeting specificity (FIG. 4B).

Next, we investigated whether we could reverse gene repression in MEGA T cells. Following transduction with RfxCas13d-DD and a CD46 targeting or non-targeting guide, we cultured MEGA T cells in either the presence or absence of TMP. On day 5, we removed or added TMP, respectively. We then tracked surface CD46 expression over 72 hours using flow cytometry, whereby we observed rapid kinetics of both induction and reversal of CD46 downregulation, and further confirmed that gene repression was completely reversed to baseline (pre-induction) levels upon TMP removal (FIG. 4C, FIG. 13C). Reversal of RfxCas13d activity was faster than induction, with an observed t1=21.36 hours post-TMP removal or t1/2=23.71 hours post-TMP addition, respectively. To further characterize the rate of target repression upon induction with TMP, we measured the kinetics of CD46 protein degradation in primary human T cells. We instantaneously blocked protein translation using cycloheximide (CHX) and conducted a flow cytometric chase assay, however, CD46 was highly stable and did not reach half-maximal expression prior to T cell apoptosis (FIG. 13D). By fitting our data to an exponential decay curve, we projected a t1/2=20.74 hours, which suggests that gene repression occurs nearly immediately after TMP addition.

Having established clean and reversible binary (ON/OFF) gene regulation, we next sought to quantitatively tune surface expression of CD46 by controlling the dosage of TMP administered to MEGA T cells. We performed TMP titration experiments and characterized CD46 dose-response using flow cytometry (FIGS. 4D-E). We observed a sigmoidal response curve and found that TMP-dependent regulation of CD46 surface expression was remarkably sensitive, with an analog-like (observed Hill coefficient=−1.425) linear dose responsive regime between approximately 1-100 nM.

We used our inducible RfxCas13d-DD to further investigate whether collateral effects affecting either off-target protein expression or cell viability (as reported by others⁵¹⁻⁵³) were present in our system. Although increasing concentrations of TMP led to increased RfxCas13d-DD on-target cleavage activity as measured by decreased CD46 expression, mCherry expression and cell viability were unaffected (FIG. 13E).

The ability of RfxCas 13d to cleave and process guide arrays is dictated by the HEPN-2 domain located at the C-terminal end of the protein⁴⁴. To evaluate whether our C-terminal RfxCas13d-DD fusion could still process guide arrays for drug-regulatable multiplexed knockdown, we extended the system to our method for suppressing inhibitory receptor upregulation in dysfunctional CAR T cells. We expanded MEGA HA-28z CAR T cells expressing a triple guide array targeting LAG3/PD-1/TIM3 with varying levels of TMP for 10 days in culture and measured surface exhaustion marker expression by flow cytometry (FIG. 4F). RfxCas13d-DD retained natural array processing capabilities and enabled simultaneous repression of all targeted exhaustion markers in a dose-dependent manner, with highly comparable knockdown efficiencies to those of the constitutive RfxCas13d system (FIG. 1G).

### Dynamic Regulation of CAR Proximal Signaling Modulates MEGA CAR T Cell Activity and Function

Recent studies from our group and others have shown that the safety and efficacy of CAR T cells can be improved by engineering new CAR designs that enable synthetic (exogenous) control of activity at the protein level^{46,62,63} Taking a receptor-independent approach to the regulation of CAR activation, we decided to utilize our inducible system to target the proximal signaling proteins LCK and ZAP70, which transduce antigen recognition signals to downstream T cell activation circuits, using a multiplexed guide array (termed “PROX array”) (FIG. 4G). By titrating levels of LCK and ZAP70 double-knockdown with varying TMP dosage, we reasoned that we could tune the upper bound of CAR activation given a strong input signal, such as in the case of an electronic amplitude limiter. Indeed, when stimulated with antigen-positive Nalm6-GD2 tumor cells, MEGA HA-28z CAR T cells exhibited a dose-dependent decrease in IL-2 secretion with increasing amounts of TMP in culture (FIG. 4H).

We next evaluated the efficiency of tonic signal attenuation by measuring expression of the T cell activation marker CD69 on MEGA HA-28z CAR T cells after 10 days of culture. The proportion of CD69+ cells decreased linearly with increasing levels of TMP (from approximately 1-100 nM) in cells expressing the PROX array, while cells expressing the non-targeting control remained unchanged (FIG. 4I). To examine off-target effects, we also quantified CD3 expression, which did not change significantly with added TMP in either group (FIG. 4J). Lastly, we measured levels of PD-1, TIM3, and LAG3 relative to non-targeting and mock controls and observed a drug-dependent reduction in the exhausted T cell surface phenotype (FIG. 4K). Altogether, these data demonstrate the ability of MEGA to fine-tune the amount of CAR signaling that can be transduced to downstream activation pathways by enforcing specific steady-state levels of LCK and ZAP70.

### Massively Multiplexed Knockdown Enables Robust Perturbation of Diverse Gene-Sets in Primary Human T Cells

We next sought to extend MEGA toward higher-order multiplexing and to target a variety of gene-sets that have been implicated in restraining T cell function within the context of anti-tumor immunity. To do so, we designed a facile two-step cloning scheme that enables fast and accurate assembly of arbitrarily long RfxCas13d guide arrays (FIG. 5A, see Methods). We used this approach to build new multiplexed arrays, which we then expressed in MEGA HA-28z CAR T cells to assess knockdown of targeted gene-sets at the RNA level.

We first targeted lactate metabolism, which has been proposed as a strategy to modulate T cell fitness and function^{64,65}. We assembled a 3-plex guide array (“LAC array”) targeting PDK1 (inhibitor of pyruvate to acetyl-CoA conversion) and the lactate dehydrogenase isozymes LDHA and LDHB by screening three different spacers per gene and concatenating the best spacers together (Figure SB, FIG. 14A). Notably, most of the spacers we tested resulted in significant gene repression, highlighting the overall robustness of RfxCas13d activity. We then expanded MEGA HA-28z CAR T cells expressing either the LAC array or a non-targeting control for 10 days in culture. We analyzed transcript data on day 10 by RT-qPCR and observed significant upregulation of LDHA and LDHB in the non-targeting control. On the other hand, cells expressing the LAC array significantly repressed LDHA, LDHB, and PDK1 to below-baseline levels (FIG. 5C).

Prior work has shown that inhibiting the PI3K/Akt axis in T cells with a small molecule inhibitor may enhance their stemness and anti-tumor activity⁶⁶. Based upon these findings, we were interested in whether we could also repress PI3K/Akt signaling and downstream glycolytic metabolism in a specific and cell-intrinsic manner using MEGA. We built a 4-plex array (“GLY array”) targeting the two main isoforms of AKT (AKT1 and AKT2) and the two main hexokinase isozymes (HK1 and HK2) that are expressed in human T cells (FIG. 5D).

After expanding MEGA HA-28z CAR T cells for 10 days in culture, we measured RNA levels and observed strong upregulation of AKT1, AKT2, and HK2 in tonically signaling CAR T cells consistent with activation of PI3K and increased glycolysis (FIG. 5E). Expression of the GLY array counteracted this upregulation as demonstrated by significant knockdown of all four targeted transcripts in the gene-set.

Our group recently discovered that targeting MED12, a core component of the mediator kinase module (CKM), with CRISPR/Cas9 knockout improves T cell effector function¹⁹. As an alternative strategy, here we designed a guide array (“CKM array”) targeting all four components of the CKM. MED12, MED13, CCNC, and CDK8 (FIG. 5F). Notably, while the CKM module was upregulated in HA-28z CAR T cells compared to mock resting cells on day 10 of culture, cells expressing the CKM array exhibited significant knockdown of all four genes below baseline levels (FIG. 5G).

After successfully demonstrating robust knockdown of diverse three- and four-gene sets, we further scaled up multiplexing to explore potential limitations in the number of genes that could be targeted in parallel. We decided to target the surfaceome of exhausted CAR T cells due to the abundance of unique and functional surface proteins with demonstrated clinical relevance. We built a 5-plex array (“SURF1 array”) by adding two new spacers targeting FAS59 and CTLA467 to our earlier guide array targeting the exhaustion markers LAG3, PDCD1, and HAVCR2. We generated MEGA HA-28z CAR T cells expressing the SURF1 array and observed significant knockdown across all 5 targeted transcripts to baseline levels or lower compared to a non-targeting control on day 10 of culture (FIG. 5H).

Finally, to stress-test the multiplex capabilities of our system, we built a 10-plex guide array (“SURF2 array”) targeting ten genes: LAG3, FAS, CD568, ENTPD169, CD4670, TRAC, B2M, CTLA4, PDCD1, and HAVCR2. We isolated bulk RNA from day 10 MEGA HA-28z CAR T cells expressing the SURF2 array and quantified transcript abundance for all ten genes compared to a non-targeting control (FIG. 5H). Remarkably, 8 out of 10 genes had significant transcript knockdown without prior optimization of spacer sequence or position. Because RfxCas13d is a shared resource across ten different crRNAs, we expected the SURF2 array to exhibit a lower knockdown efficiency than SURF1 due to competition between guides for limited Cas13 protein, as noted in previous Cas9 retroactivity studies^{40,58}. Indeed, for all genes targeted in the SURF1 array, we observed slightly lower knockdown efficiency by SURF2. However, we did not observe any correlation between RfxCas13d activity and target transcript abundance across all the gene-sets that we perturbed using MEGA (FIG. 14B). Collectively, our data highlights the ability of MEGA to knock down putative functional gene-sets ranging from 3 to 10 targets at once and offers a powerful approach to investigate protein complexes and pathways that remain unexplored in primary human T cells.

### Whole-Pathway Disruption of Purinergic Signaling Modulates Environmental Metabolic Flux and Improves Effector Function in Dysfunctional CAR T Cells

Solid tumors remain challenging for T cell-based therapies in part due to the accumulation of immunosuppressive metabolites such as adenosine (ADO) within the tumor microenvironment⁷¹. Although ATP and ADO are both present at high (micromolar) levels in solid tumor milieu⁷², the purinergic pathway rapidly converts inflammatory ATP signals into immunosuppressive ADO signals through a cascade of enzymatic reactions that involves four key surface proteins: CD39, CD73, and the adenosine receptors A2AR and A2BR (FIG. 6A).

Having established a robust method for gene-set perturbation, we wanted to explore whether utilizing our platform to disrupt the entire purinergic signaling cascade would slow the generation of ADO while accumulating ATP to enhance the anti-tumor activity of dysfunctional CAR T cells. We built a four-gene guide array (PURI array) targeting ENTPD1 (CD39), NTSE (CD73), ADORA2A (A2AR), and ADORA2B (A2BR) (FIG. 6B). To validate array functionality, we manufactured MEGA HA-28z CAR T cells expressing either the PURI array or a non-targeting guide and analyzed RNA transcript abundance on day 10 of culture.

We observed significant knockdown across all four targeted transcripts as measured by RT-qPCR (FIG. 6C). We next performed an ATP spike-in experiment to measure extracellular concentrations of ATP, AMP, and ADO and evaluate whether the PURI array modulated metabolic flux within the pathway (FIGS. 6D-F). MEGA HA-28z CAR T cells expressing the PURI array accumulated significantly more ATP and generated significantly less AMP and less ADO than the non-targeting control. We also measured ATP and AMP levels at regular time intervals to calculate effective rate constants (FIGS. 6G-H). Expression of the PURI array significantly slowed ATP hydrolysis and AMP production in comparison to the non-targeting control.

Encouraged by these results, we set up co-culture assays with Nalm6-GD2 tumor cells to investigate whether disruption of the purinergic pathway could improve effector function in dysfunctional CAR T cells. While we did not observe significant changes to cytokine secretion from baseline tonic signaling, antigen-stimulated MEGA HA-28z CAR T cells expressing the PURI array secreted significantly greater amounts of both IFN-γ and IL-2 in comparison to the non-targeting control (FIGS. 6I-J). Furthermore, PURI array knockdown significantly boosted in vitro tumor killing and T cell activation and proliferation in response to antigen stimulation (FIGS. 6K-L).

### MEGA Enables Metabolic Engineering of CAR T Cells to Improve Anti-Tumor Activity, Modulate T Cell Differentiation, and Mitigate Exhaustion

We utilized MEGA to knock down four genes: AKT1, AKT2, HK1, HK2. These genes encode redundant protein isoforms that are involved in PI3K/Akt-driven aerobic glycolysis, a metabolic process that is kickstarted by CAR/TCR activation and leads to T cell effector differentiation and eventual exhaustion/dysfunction (FIG. 15A). After disrupting aerobic glycolysis using MEGA, we observed a healthier (less exhausted) surface phenotype in HA-28z CAR T cells (FIGS. 15B-E). We also saw a decrease in molecular/metabolic signatures associated with T cell dysfunction (FIGS. 15F-I). In functional assays, these cells were less effector-like, were able to proliferate better in culture, and were able to sustain anti-tumor activity in a repeat challenge assay (FIG. 15J). We then did a head-to-head comparison with state-of-the-art Cas9 knockout. We electroporated HA-28z CAR T cells with Cas9 RNPs targeting the same four genes (FIGS. 15K-L). In contrast to MEGA, the Cas9 knockout worsened the ability of T cells to expand in culture and control tumor (FIGS. 15M-N). Upon examining the transcriptome, we saw that these cells also downregulated p53 and upregulated DNA damage/G2M checkpoint pathways (FIGS. 150-R), which is consistent with previous findings showing that Cas9 is genotoxic. Importantly, we didn't see differential enrichment of these pathways in MEGA CAR T cells (FIGS. 150-R).

MEGA can be Used to Target and Degrade Specific Transcripts in Primary Human T Cells without Off-Target Collateral Activity

Recent work has suggested that on-target RfxCas13d activity triggers off-target collateral activity (nonspecific cleavage of RNAs) We performed the experiments to explore whether such off-target collateral activity was present in the MEGA system. RfxCas13d RNA-level and protein-level were tested to evaluate collateral activity resulting from on-target B2M cleavage in primary human T cells from n=2 donors (FIG. 16A). No off-target collateral activities were observed in either RNA-level or protein-level of mCherry-P2A-RfxCas13d in primary human MEGA T cells (FIGS. 16B-H).

### MEGA can be Used to Improve the Anti-Tumor Efficacy of (CAR) T Cells In Vivo

The in vitro assay showed that the GLY array targeting HK1, HK2, AKT1, and AKT2 prevented CAR T cells from acquiring a dysfunctional phenotype and terminal effector differentiation through disruption of aerobic glycolysis (FIG. 15). Next, we performed in vivo tumor challenge experiments in mice to validate our in vitro findings. We prepared these same cells and injected them into mice bearing antigen-positive tumor (Nalm6-GD2) (FIG. 17A). The GLY array improved the anti-tumor response of MEGA HA-28z CAR T cells in vivo when compared to the non-targeting control as measured by tumor bioluminescence imaging (BLI) over 12 days (FIGS. 17B-C).

### Massively-Multiplexed Knockdown of Ten Genes in MEGA HA-28z CAR T Cells Broadly and Robustly Reprograms the Transcriptome at the Single-Cell Level

We performed single cell RNA sequencing (scRNA-seq) of MEGA HA-28z CAR T cells expressing either the SURF2 array (FIG. 5) or a non-targeting guide to explore the effect of ten-gene knockdown on global gene expression in single cells. We ran unsupervised clustering of these cells according to their global gene expression signatures and visualized the resulting clusters in a two-dimensional UMAP plot, where we saw clear separation of the cells by sample ID (FIGS. 18A-C). This indicates that: 1) the SURF2 cells were similar to each other, suggesting robust and uniform perturbation by MEGA; and 2) the SURF2 cells were significantly different from the non-targeting control cells, suggesting that ten-gene perturbation leads to broad and global transcriptomic changes. When we visualize gene expression levels of the most significantly differentially expressed genes (SURF2 vs. NT) on the UMAP plot, we see that SURF2 increases the expression of genes associated with glycolysis and mitophagy while decreasing the expression of mitochondrial genes (FIGS. 18B-C).

### DISCUSSION

Multiplexed silencing of endogenous genes has been a major focus of recent studies seeking to improve T cell therapies or develop allogeneic “off-the-shelf” modalities^{3,25,27} Despite an ever-expanding list of promising gene targets, scaling up multiplexed perturbation beyond three genes quickly becomes intractable with current methods. As such, it has essentially been impossible to manipulate larger gene-sets, such as metabolic pathways (which often include several redundant or compensatory protein isoforms) or functional heteromeric protein complexes. Here, we show repression of up to 10 endogenous genes at once from a single guide array without DNA double-strand break induction. We also demonstrate robust knockdown of multiple protein isoforms, a multi-unit protein complex, a signaling cascade, and an entire immunosuppressive metabolic pathway to reshape the chemical composition of the tumor microenvironment. We believe MEGA will pave the way for further metabolic pathway discovery and engineering work in primary human T cells, which is particularly exciting as these studies have only been possible in microbes and plants thus far.

Unlike Cas9-based tools, MEGA does not incur significant costs, such as increased genotoxic risk or decreased editing specificity, with increasing plexity. Our results also indicate that MEGA does not cause observable off-target or collateral cleavage effects that may hinder T cell viability. While higher-order multiplexing (>10 genes) may require optimization of spacer position or sequence for robust repression of all targeted genes, we describe a rapid and facile assembly method of arbitrarily long guide arrays to accelerate this process.

By applying MEGA to an established model of primary human CAR T cell dysfunction, we demonstrate that MEGA does not disrupt CAR expression or signaling and is compatible with T cell anti-tumor functionality. Tonically signaling MEGA CAR T cells are able to robustly suppress the expression of multiple inhibitory receptors, despite active transcriptional^{7, 46} and epigenetic⁴⁷ upregulation of these genes by cell-intrinsic exhaustion programs. Furthermore, our combinatorial CRISPR screen helps elucidate synergistic relationships between putative exhaustion-related genes described in previous CRISPR/Cas9 single knockout screens, and we show that paired knockdown of these newly identified gene combinations broadly enhances anti-tumor activity. Specifically, dual knockdown of CBLB+FAS best preserved the ability of dysfunctional CAR T cells to control tumor growth against multiple rechallenges, which highlights a potential mechanism of synergy that warrants further investigation.

Although our proof-of-concept study focuses on a small subset of gene pairs, MEGA is highly scalable and can be used to screen larger subsets of functionally-related gene pairs (e.g., metabolic gene-sets) or even a comprehensive 2D genome-wide set. For such applications, pre-validated spacer sequences should reduce false-negatives and improve signal-to-noise to allow for other modes of analyses, such as quantitative genetic interaction (GI) mapping⁵⁵. While spacer-dependent variability is common to all CRISPR systems, and especially dCas9-based tools²⁹, our group and others have begun to develop more accurate algorithms to predict and generate highly efficient RfxCas13d spacer sequences^{48, 50, 73.}

To date, no CRISPR system has been stably expressed in primary human T cells for therapeutic applications³. A recent landmark Phase 1 clinical trial was the first to use Cas9-edited T cells for patient infusion²⁶, however, this and other preclinical studies¹⁴ have relied exclusively on transient Cas9 electroporation to introduce permanent edits to the genome. One group showed that electroporation of recombinant Cas13d can mediate knockdown of a single gene in T cells⁷⁴, however, the scope of this method is limited to highly transient perturbations within a specific timeframe during manufacturing, and it is unclear whether knockdown efficacy is robust for other endogenous genes. Lastly, Cas9 fusion effectors either approach or exceed the packaging limit of conventional lentiviruses^{75,76}, and weak expression levels of these constructs in primary human T cells are impractical for sustained therapeutic applications^{18,77}. In contrast to these studies, we report for the first time a powerful genetically-encoded CRISPR system that enables tuning of both the magnitude and duration of multiple genetic perturbations at any point across the lifetime of the cell.

In contrast to binary and irreversible gene knockout methods, precise control over transcript expression may enhance the safety and efficacy of adoptive T cell therapies. Specifically, regulating CAR activation has been of great interest, though current cell-intrinsic methods rely on laborious protein engineering of the receptor itself—the results of which may vary drastically due to inherent differences between receptor architectures that affect function¹¹. In our work, we show that MEGA can be used to tune the input-output function of the CAR independently of the receptor used by linking dosage of TMP to the expression of the proximal signaling molecules LCK and ZAP70. Our amplitude limiter functions over a variety of input signal strengths, from basal tonic signaling to antigen stimulation by tumor cells.

Beyond regulation of CAR signaling, MEGA enables precise tuning of gene-set expression and pathway activity in T cells even after manufacturing. This may help strike a “sweet-spot” between T cell dysfunction and excessive function or autoimmunity in patients post-infusion^{78.79}. For example, in this study we confirm prior findings that IRF4 is essential for T cell proliferation, despite its role in driving exhaustion. While extreme high or low levels of IRF4 may inhibit T cell function, enforcing an intermediate level of expression may be beneficial in the context of anti-tumor immunity^{7, 31, 35} Recent work has also shown that although lower TET2 expression correlates with enhanced T cell memory, complete genetic ablation of TET2 can generate hyperproliferative T cells with genomic instability⁸⁰ Notably, a method to specifically tune the metabolism of engineered T cells within patients has been an established unmet need¹⁰-thus, MEGA may open the door to a new suite of potential metabolic interventions for cancer immunotherapy.

Finally, the transient and reversible nature of MEGA expands the current genetic perturbation space to include previously-unexplored temporal manipulation of specific genes or gene-sets. For example, reversible knockdown of B2M/HLA could be developed as an alternative to traditional “safety switches” by leveraging immune rejection to regulate T cell persistence within a patient. On the other hand, conditional degradation of specific mRNA or long noncoding RNA (lncRNA) transcripts during highly dynamic T cell processes (such as activation, differentiation, and exhaustion) may offer new biological and therapeutic insights.

In summary, we developed MEGA, a synthetic biology-driven platform for versatile transcriptomic regulation in primary human T cells using CRISPR/Cas13d. Our work highlights a variety of T cell engineering applications made possible with MEGA and addresses key limitations posed by state-of-the-art CRISPR/Cas9 gene-editing technologies (FIG. 7). We envision MEGA as an invaluable addition to the synthetic immunology toolkit: engineered MEGA T cells are equipped with a vast array of novel capabilities spanning applications from basic biological discovery to improved cell-based immunotherapies for cancer and beyond.

### REFERENCES

1. Brown, C. E. & Mackall, C. L. CAR T cell therapy: inroads to response and resistance. Nat. Rev. Immunol. 19, 73-74 (2019).
2. Finck, A. V., Blanchard, T., Roselle, C. P., Golinelli, G. & June, C. H. Engineered cellular immunotherapies in cancer and beyond. Nat. Med. 28, 678-689 (2022).
3. Dimitri, A., Herbst, F. & Fraietta, J. A. Engineering the next-generation of CAR T-cells with CRISPR-Cas9 gene editing. Mol. Cancer 21, 78 (2022).
4. Majzner, R. G. et al. GD2-CAR T cell therapy for H3K27M-mutated diffuse midline gliomas. Nature 603, 934-941 (2022).
5. Larson, R. C. & Maus, M. V. Recent advances and discoveries in the mechanisms and functions of CAR T cells. Nat. Rev. Cancer 21, 145-161 (2021).
6. Majzner, R. G. & Mackall, C. L. Clinical lessons learned from the first leg of the CAR T cell journey. Nat. Med. 25, 1341-1355 (2019).
7. Lynn, R. C. et al. c-Jun overexpression in CAR T cells induces exhaustion resistance. Nature 576, 293-300 (2019).
8. Fraietta, J. A. et al. Determinants of response and resistance to CD19 chimeric antigen receptor (CAR) T cell therapy of chronic lymphocytic leukemia. Nat. Med. 24, 563-571 (2018).
9. Pauken, K. E. & Wherry, E. J. Overcoming T cell exhaustion in infection and cancer. Trends Immunol. 36, 265-276 (2015).
10. O'Sullivan, D., Sanin, D. E., Pearce, E. J. & Pearce, E. L. Metabolic interventions in the immune response to cancer. Nat. Rev. Immunol. 19, 324-335 (2019).
11. Guedan, S., Calderon, H., Posey, A. D. & Maus, M. V. Engineering and Design of Chimeric Antigen Receptors. Mol. Ther.-Methods Clin. Dev. 12, 145-156 (2019).
12. Chen, Z. et al. In vivo CD8⁺ T cell CRISPR screening reveals control by Flil in infection and cancer. Cell 184, 1262-1280.e22 (2021).
13. Wei, J. et al. Targeting REGNASE-1 programs long-lived effector T cells for cancer therapy. Nature 576, 471-476 (2019).
14. Shifrut, E. et al. Genome-wide CRISPR Screens in Primary Human T Cells Reveal Key Regulators of Immune Function. Cell 175, 1958-1971.e15 (2018).
15. Dong, M. B. et al. Systematic Immunotherapy Target Discovery Using Genome-Scale In Vivo CRISPR Screens in CD8 T Cells. Cell 178, 1189-1204.e23 (2019).
16. Zhao, H. et al. Genome-wide fitness gene identification reveals Roquin as a potent suppressor of CD8 T cell expansion and anti-tumor immunity. Cell Rep. 37, 110083 (2021).
17. Belk, J. A. et al. Genome-wide CRISPR screens of T cell exhaustion identify chromatin remodeling factors that limit T cell persistence. 2022.04.20.488974 Preprint at https://doi.org/10.1101/2022.04.20.488974 (2022).
18. Schmidt, R. et al. CRISPR activation and interference screens decode stimulation responses in primary human T cells. Science 375, eabj4008 (2022).
19. Freitas, K. A. et al. Enhanced T cell effector activity by targeting the Mediator kinase module. Science 378, eabn5647 (2022).
20. Chen, J. et al. NR4A transcription factors limit CAR T cell function in solid tumours. Nature 567, 530-534 (2019).
21. Khan, O. et al. TOX transcriptionally and epigenetically programs CD8⁺ T cell exhaustion. Nature 571, 211-218 (2019).
22. Fraietta, J. A. et al. Disruption of TET2 promotes the therapeutic efficacy of CD19-targeted T cells. Nature 558, 307-312 (2018).
23. Rupp, L. J. et al. CRISPR/Cas9-mediated PD-1 disruption enhances anti-tumor efficacy of human chimeric antigen receptor T cells. Sci. Rep. 7, 737 (2017).
24. LaFleur, M. W. et al. PTPN2 regulates the generation of exhausted CD8⁺ T cell subpopulations and restrains tumor immunity. Nat. Immunol. 20, 1335-1347 (2019).
25. Ren, J. et al. Multiplex Genome Editing to Generate Universal CAR T Cells Resistant to PD1 Inhibition. Clin. Cancer Res. 23, 2255-2266 (2017).
26. Stadtmauer, E. A. et al. CRISPR-engineered T cells in patients with refractory cancer Science 367, eaba7365 (2020). 22
27. Webber, B. R. et al. Highly efficient multiplex human T cell engineering without double-strand breaks using Cas9 base editors. Nat. Commun. 10, 5222 (2019).
28. Eyquem, J. et al. Targeting a CAR to the TRAC locus with CRISPR/Cas9 enhances tumour rejection. Nature 543, 113-117 (2017).
29. Wang, H, La Russa, M. & Qi, L. S. CRISPR/Cas9 in Genome Editing and Beyond. Annu. Rev. Biochem. 85, 227-264 (2016).
30. Komor, A. C., Kim, Y. B, Packer, M. S., Zuris, J. A. & Liu, D. R. Programmable editing of a target base in genomic DNA without double-stranded DNA cleavage. Nature 533, 420-424 (2016).
31. Man, K. et al. Transcription Factor IRF4 Promotes CD8⁺ T Cell Exhaustion and Limits the Development of Memory-like T Cells during Chronic Infection. Immunity 47, 1129-1141.e5 (2017).
32. Odorizzi, P. M., Pauken, K. E., Paley, M. A., Sharpe, A. & Wherry, E. J. Genetic absence of PD-1 promotes accumulation of terminally differentiated exhausted CD8⁺ T cells. J. Exp. Med. 212, 1125-1137 (2015).
33. Collier, J. L., Weiss, S. A., Pauken, K. E., Sen, D. R. & Sharpe, A. H. Not-so-opposite ends of the spectrum: CD8⁺ T cell dysfunction across chronic infection, cancer and autoimmunity. Nat. Immunol. 22, 809-819 (2021)
34. Wei, J. et al. PD-1 silencing impairs the anti-tumor function of chimeric antigen receptor modified T cells by inhibiting proliferation activity. J. Immunother. Cancer 7, 209 (2019).
35. Harberts, A. et al. Interferon regulatory factor 4 controls effector functions of CD8⁺ memory T cells. Proc. Natl. Acad. Sci. 118, e2014553118 (2021).
36. Nahmad, A. D. et al. Frequent Aneuploidy in Primary Human T Cells Following CRISPR- Cas9 cleavage. 2021.08.20.457092 doi: 10.1101/2021.08.20.457092.
37. Kosicki, M., Tomberg, K. & Bradley, A. Repair of double-strand breaks induced by CRISPR-Cas9 leads to large deletions and complex rearrangements. Nat. Biotechnol. 36, 765-771 (2018).
38. Enache, O. M. et al. Cas9 activates the p53 pathway and selects for p53-inactivating mutations. Nat. Genet. 52, 662-668 (2020)
39. Kempton, H. R. & Qi, L. S. When genome editing goes off-target. Science 364, 234-236 (2019).
40. McCarty, N. S., Graham, A. E., Studena, L. & Ledesma-Amaro, R. Multiplexed CRISPR technologies for gene editing and transcriptional regulation. Nat. Commun. 11, 1281 (2020).
41. Park, S. & Beal, P. A. Off-Target Editing by CRISPR-Guided DNA Base Editors. Biochemistry 58, 3727-3734 (2019).
42. Konermann, S. et al. Transcriptome Engineering with RNA-Targeting Type VI-D CRISPR Effectors. Cell 173, 665-676.e14 (2018).
43. Yan, W. X. et al. Cas13d Is a Compact RNA-Targeting Type VI CRISPR Effector Positively Modulated by a WYL-Domain-Containing Accessory Protein. Mol. Cell 70, 327-339.e5 (2018).
44. Zhang, B. et al. Two HEPN domains dictate CRISPR RNA maturation and target cleavage in Cas13d. Nat. Commum. 10, 2544 (2019).
45. Long, A. H. et al. 4-1BB costimulation ameliorates T cell exhaustion induced by tonic signaling of chimeric antigen receptors. Nat. Med. 21, 581-590 (2015)
46. Weber, E. W. et al. Transient rest restores functionality in exhausted CAR-T cells through epigenetic remodeling. Science 372, (2021).
47. Gennert, D. G. et al. Dynamic chromatin regulatory landscape of human CAR T cell exhaustion. Proc. Natl. Acad. Sci. 118, e2104758118 (2021).
48. Abbott, T. R. et al. Development of CRISPR as an Antiviral Strategy to Combat SARS-CoV-2 and Influenza. Cell 181, 865-876.e12 (2020).
49. Wherry, E. J. & Kurachi, M. Molecular and cellular insights into T cell exhaustion. Nat. Rev. Immunol. 15, 486-499 (2015).
50. Wessels, H.-H. et al. Massively parallel Cas13 screens reveal principles for guide RNA design. Nat. Biotechnol. (2020) doi: 10.1038/s41587-020-0456-9.
51. Özcan, A. et al. Programmable RNA targeting with the single-protein CRISPR effector Cas7-11. Nature 597, 720-725 (2021).
52. Kelley, C. P., Haerle, M. C. & Wang, E. T. Negative autoregulation mitigates collateral RNase activity of repeat-targeting CRISPR-Cas13d in mammalian cells. 2021.12.20.473384 Preprint at https://doi.org/10.1101/2021.12.20 473384 (2021). 23
53. Tong, H. et al. High-fidelity Cas13 variants for targeted RNA degradation with minimal collateral effects. Nat. Biotechnol. 1-12 (2022) doi: 10.1038/s41587-022-01419-7.
54. Marques, J. T. & Williams, B. R. G. Activation of the mammalian immune system by siRNAs. Nat. Biotechnol. 23, 1399-1405 (2005)
55. Horlbeck, M. A. et al. Mapping the Genetic Landscape of Human Cells. Cell 174, 953-967.e22 (2018).
56. Wessels, H.-H. et al. Efficient combinatorial targeting of RNA transcripts in single cells with Cas13 RNA Perturb-seq. Nat. Methods 20, 86-94 (2023).
57. Katagiri, T. et al. JunB plays a crucial role in development of regulatory T cells by promoting IL-2 signaling. Mucosal Immunol. 12, 1104-1117 (2019).
58. Ren, J. et al. A versatile system for rapid multiplex genome-edited CAR T cell generation. Oncotarget 8, 17002-17011 (2017).
59. Tschumi, B. O. et al. CART cells are prone to Fas- and DR5-mediated cell death. J. Immunother. Cancer 6, 71 (2018).
60. Hooper, K. et al. Knockout of CBLB Greatly Enhances Anti-Tumor Activity of CAR T Cells. Blood 132, 338 (2018).
61. Banaszynski, L. A., Chen, L., Maynard-Smith, L. A., Ooi, A. G. L. & Wandless, T. J. A Rapid, Reversible, and Tunable Method to Regulate Protein Function in Living Cells Using Synthetic Small Molecules. Cell 126, 995-1004 (2006).
62. Labanieh, L. et al. Enhanced safety and efficacy of protease-regulated CAR-T cell receptors. Cell 185, 1745-1763.e22 (2022).
63. Li, H.-S. et al. High-performance multiplex drug-gated CAR circuits. Cancer Cell 40, 1294-1305.e4 (2022).
64. Quinn, W. J. et al. Lactate Limits T Cell Proliferation via the NAD (H) Redox State. Cell Rep. 33, 108500 (2020).
65. Menk, A. V. et al. Early TCR Signaling Induces Rapid Aerobic Glycolysis Enabling Distinct Acute T Cell Effector Functions. Cell Rep. 22, 1509-1521 (2018).
66. Klebanoff, C. A. et al. Inhibition of AKT signaling uncouples T cell differentiation from expansion for receptor-engineered adoptive immunotherapy. JCI Insight 2, e95103.
67. Wei, S. C. et al. Distinct Cellular Mechanisms Underlie Anti-CTLA-4 and Anti-PD-1 Checkpoint Blockade. Cell 170, 1120-1133.e17 (2017).
68. Blaize, G. et al. CD5 signalosome coordinates antagonist TCR signals to control the generation of Treg cells induced by foreign antigens. Proc. Natl. Acad. Sci. 117, 12969-12979 (2020).
69. Gupta, P. K. et al. CD39 Expression Identifies Terminally Exhausted CD8⁺ T Cells. PLOS Pathog. 11, e1005177 (2015).
70. Kemper, C. et al. Activation of human CD4+ cells with CD3 and CD46 induces a T-regulatory cell 1 phenotype. Nature 421, 388-392 (2003).
71. Vijayan, D., Young, A., Teng, M. W. L. & Smyth, M. J. Targeting immunosuppressive adenosine in cancer. Nat. Rev. Cancer 17, 709-724 (2017).
72. Allard, B., Allard, D., Buisseret, L. & Stagg, J. The adenosine pathway in immuno-oncology. Nat. Rev. Clin. Oncol. 17, 611-629 (2020).
73. Wei, J. et al. Deep learning of Cas13 guide activity from high-throughput gene essentiality screening. 2021.09.14.460134 Preprint at https://doi.org/10.1101/2021.09.14.460134 (2021).
74. Méndez-Mancilla, A. et al. Chemically modified guide RNAs enhance CRISPR-Cas13 knockdown in human cells. Cell Chem. Biol. (2021) doi: 10.1016/j.chembiol.2021.07.011.
75. Xu, X. et al. Engineered miniature CRISPR-Cas system for mammalian genome regulation and editing. Mol. Cell 81, 4333-4345.e4 (2021).
76. Nuñez, J. K. et al. Genome-wide programmable transcriptional memory by CRISPR-based epigenome editing. Cell 184, 2503-2519.e17 (2021).
77. Chen, X. et al. Functional Interrogation of Primary Human T Cells via CRISPR Genetic Editing. J. Immunol. 201, 1586-1598 (2018).
78. Grebinoski, S. et al. Autoreactive CD8⁺ T cells are restrained by an exhaustion-like program that is maintained by LAG3. Nat. Immunol. 1-10 (2022) doi: 10.1038/s41590-022-01210-5.
79. Cruz, A. C. et al. Fas/CD95 prevents autoimmunity independently of lipid raft localization and efficient apoptosis induction. Nat. Commum. 7, 13895 (2016).
80. Jain, N. et al. TET2 guards against unchecked BATF3-induced CAR T cell expansion. Nature 1-8 (2023) doi: 10.1038/s41586-022-05692-z. 24
81. Bray, N. L., Pimentel, H., Melsted, P. & Pachter, L. Near-optimal probabilistic RNA-seq quantification. Nat. Biotechnol. 34, 525-527 (2016).
82. Love, M. I. et al. Tximeta: Reference sequence checksums for provenance identification in RNA-seq. PLOS Comput. Biol. 16, e1007664 (2020).
83. Love, M. I., Huber, W. & Anders, S. Moderated estimation of fold change and dispersion for RNA-seq data with DESeq2. Genome Biol. 15, 550 (2014).
84. Zhu, A., Ibrahim, J. G. & Love, M. I. Heavy-tailed prior distributions for sequence count data: removing the noise and preserving large differences. Bioinformatics 35, 2084-2092 (2019).
85. Wang, B. et al. Integrative analysis of pooled CRISPR genetic screens using MAGeCKFlute. Nat. Protoc. 14, 756-780 (2019).
86. Langmead, B. & Salzberg, S. L. Fast gapped-read alignment with Bowtie 2. Nat. Methods 9, 357-359 (2012).
87. Doench, J. G. et al. Optimized sgRNA design to maximize activity and minimize off-target effects of CRISPR-Cas9. Nat. Biotechnol. 34, 184-191 (2016).
88. Li, W. et al. MAGeCK enables robust identification of essential genes from genome-scale CRISPR/Cas9 knockout screens. Genome Biol. 15, 554 (2014).

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

TABLE 1
Exemplary crRNA spacer sequences.

| NAME | SEQ |
|---|---|
| HAVCR2-1 | CGAAGATAAGAGCCAGAGCCAGC |
| HAVCR2-2 | CTTTCATCAGTCCTGAGCACCAC |
| HAVCR2-3 | CAGAAGAAAAGTCAGAGGACACC |
| LAG3-1 | GAGCAGTTCAAAATGACCCAGTC |
| LAG3-2 | GTCGCCATTGTCTCCAGTCACCA |
| LAG3-3 | AGAAACAGCAAGCCCAGGAACTG |
| PDCD1-1 | AGTCCACAGAGAACACAGGCACG |
| PDCD1-2 | GGTACCAGTTTAGCACGAAGCTC |
| PDCD1-3 | GAAAGACAATGGTGGCATACTCC |
| NT | GTAACTACTCGAGACGCCTATT |
| FAS | AGCAACAGACGTAAGAACCAGAG |
| CTLA4 | CCATCATGTAGGTTGCCGCACAG |
| CD5 | AAACCAAAGGAACATGACCAGCC |
| CD39 | ATAGTTGATAGTAATCCAGCCAT |
| CD46 | AGACAATTGTGTCGCTGCCATCG |
| TRAC | GCAAAGTCAGATTTGTTGCTCCA |
| B2M | CCACAAAAGCTAGAGGAAGCCAG |
| CBLB | ACCATTATCACAAGACCGAACAG |

TABLE 2
Exemplary crRNA spacer sequences.

| GENE ID | GUIDE SEQ |
|---|---|
| TOX | AAGAATAACGCATAGGCAGACAC |
| TOX | AGAATAACGCATAGGCAGACACA |
| TOX | CAATTTTAGAGACTTCGCCAAAG |
| TOX2 | ACGATTTTGGACACGTCACCGAA |
| TOX2 | AGATCTTGAAATGCACTTCCGAC |
| TOX2 | TCACCATCAAACTTGCCGCCGTG |
| NR4A1 | CCATAGTAGTCAGAGCCACTGGA |
| NR4A1 | ACATCGACAAGCAAGCTGTGCAG |
| NR4A1 | CCCAACAGACGTGACAGGCAGCT |
| NR4A2 | GAACCACTTCTTTGACCATOCCA |
| NR4A2 | AACCACTTCTTTGACCATOCCAA |
| NR4A2 | AGCTGATTCAAAAAGCAGGTCTT |
| NR4A3 | AAGTCTTTAATAGAGTCGAGCCA |
| NR4A3 | AAAAGTCTTTAATAGAGTCGAGC |
| NR4A3 | AGAATTGTTGCACATGCTCAGCA |
| TET2 | AAATGAAATCTAGTGCCACACAG |
| TET2 | GAACAGAATTCTTCACCAGACGC |
| TET2 | AAATTATTGAGAACAGAAGCAGC |
| IRF4 | GCACAAGCATAAAAGGTTCCTGT |
| IRF4 | AATAATATAGTTGTCTGGCTAGC |
| IRF4 | CTCCAAAGCATAGAGTCACCTGG |
| JUNB | ACCAGAAAAGTAGCTGCCGCCAC |
| JUNB | CCAGAAAAGTAGCTGCCGCCACC |
| JUNB | TGTTCCATTTTAGTGCACATCCG |
| BATF3 | ACAAAGTTCATAGGGCAGAGCAG |
| BATF3 | CAAAGTTCATAGGGCAGAGCAGC |
| BATF3 | GGCACAAAGTTCATAGGGCAGAG |
| DHX37 | GATTTCTTTAAGCAGCACACCAT |
| DHX37 | TTCATAGAGAAACTGAGGCACCT |
| DHX37 | ACCGCAGATGAATACAGCCTGTA |
| FLI1 | AGAATTCATGTTATTGCCCCAAG |
| FLI1 | CAGAATTCATGTTATTGCCCCAA |
| FLI1 | TCATAGTAATAACGGAGGGCCCG |
| ZC3H12A | AATCTGTCGTCATAGCACACCAC |
| ZC3H12A | TCCAGAAACCAGTTCACTGCCAG |
| ZC3H12A | CACACAGCTTAGTATACACGCTG |
| SOCS1 | ACCATCCTACAGAAGGGGCCAGC |
| SOCS1 | ACTGCATTGTCGGCTGCCACCTG |
| SOCS1 | CAGTAGAATCCGCAGGCGTCCAG |
| TCEB2 | ATCATCCAAGAGTTGGTCATCCT |
| TCEB2 | ATGCGCTTCAGTTCGAACACCGT |
| TCEB2 | TTGGTCATCCTTGTACAGCCGCT |
| PDCD1 | AGTCCACAGAGAACACAGGCACG |
| PDCD1 | CCACAGAGAACACAGGCACGGCT |
| PDCD1 | GAAAGACAATGGTGGCATACTCC |
| HAVCR2 | ACCATTIGAAAATTAAAGCGCCG |
| HAVCR2 | CCGAAGATAAGAGCCAGAGCCAG |
| HAVCR2 | ACCTTGTAAGTAGTAGCAGCAGC |
| LAG3 | GAGCAGTTCAAAATGACCCAGTC |
| LAG3 | CAGTTCAAAATGACCCAGTCGGA |
| LAG3 | GCAGAAAATCGTCTTGGTCGCCA |
| CTLA4 | CCATCATGTAGGTTGCCGCACAG |
| CTLA4 | CGCTGAAATCCAAGGCAAGCCAT |
| CTLA4 | AAAAACAGGAGAGTGCAGGGCCA |
| TIGIT | ACATTGTAAGATGATAGAGCCAC |
| TIGIT | GACATTGAAGTAGTCATGCAGCT |
| TIGIT | TCTTTCTAGTCAACGCGACCACC |
| FAS | AGCAACAGACGTAAGAACCAGAG |
| FAS | CAGATAAATTTATTGCCACTGTT |
| FAS | TAGCAACAGACGTAAGAACCAGA |
| TRAC | AAAATCGGTGAATAGGCAGACAG |
| TRAC | TCATGAGCAGATTAAACCCGGCC |
| TRAC | GCAAAGTCAGATTTGTTGCTCCA |
| CBLB | AAGAGAATTTGCTAACGGACCAG |
| CBLB | ACCATTATCACAAGACCGAACAG |
| CBLB | TTAAATATATGCTTAAGTCAGGG |
| RASA2 | ATATTTAATCGAAGAGACCCCAG |
| RASA2 | CACTCAAAATGTAAGCAGCTGAG |
| RASA2 | TAGACTGCAATAGTTCCACAAGC |
| PTPN2 | CCAGCCAACAAAAGCGCCAACCA |
| PTPN2 | AAAACAAGACAAGTGTCTACCAG |
| PTPN2 | AGAAATTGAGAAATGAAGCTGGT |
| NONTARGETING | TCACCAGAAGCGTACCATACTCG |
| NONTARGETING | TAACCCGATGAGCTACCCAGTAG |
| NONTARGETING | CGCTAATGCACTCAATCCCGAGG |
| NONTARGETING | CCTGACGCGACATATCAGCTAAG |
| NONTARGETING | AGTAGGCCGGGGGTGTAGACCTT |
| NONTARGETING | CTAATCGGCTTCAACGTGCCGCA |
| NONTARGETING | CTATTGAGGCATTGACTGATGCC |
| NONTARGETING | AGGACATATGCCCTACCTCCATG |

TABLE 3
Cloning Primers.

| | |
|---|---|
| PDCD1- | tcttgtggaaaggacgaaacaccgaacccctacca |
| 3_IRF4- | actggtcggggtttgaaacGAAAGACAATGGTGGC |
| 2 | ATACTCCaacccctaccaactggtcggggtttgaa |
| | acAATAATATAGTTGTCTGGCTAGCtttttttgaa |
| | ttcgctagctaggt |
| vt05- | tcttgtggaaaggacgaaacaccgAACCCCTACCA |
| 1_10-1 | ACTGGTCGGGGTTTGAAACgagcagttcaaaatga |
| | cccagtcCAAGTAAACCCCTACCAACTGGTCGGGG |
| | TTTGAAACAGCAACAGACGTAAGAACCAGAG |
| v105-2 | CTGTGCGGCAACCTACATGATGGGTTTCAAACCCC |
| | GACCAGTTGGTAGGGGTTTACTTGCTCTGGTTCTT |
| | ACGTCTGTTGCT |
| v105- | CCATCATGTAGGTTGCCGCACAGCAAGTAAACCCC |
| 3_10-5 | TACCAACTGGTCGGGGTTTGAAACggtaccagttt |
| | agcacgaagctcCAAGTAAACCCCTACCAACTGGT |
| | CGGGGTTTGAAACcagaagaaaagtcagaggacac |
| | ctttttttgaattcgctagctaggt |
| vt10-2 | ATGGCTGGATTACTATCAACTATGTTTCAAACCCC |
| | GACCAGTTGGTAGGGGTTTACTTGGGCTGGTCATG |
| | TTCCTTTGGTTTGTTTCAAACCCCGACCAGTTGGT |
| | AGGGGTTTACTTGCTCTGGTTCTTACGTCTGTTGC |
| | T |
| vt10-3 | ATAGTTGATAGTAATCCAGCCATCAAGTAAACCCC |
| | TACCAACTGGTCGGGGTTTGAAACCAGACAATTGT |
| | GTCGCTGCCATCGCAAGTAAACCCCTACCAACTGG |
| | TCGGGGTTTGAAACGCAAAGTCAGATTTGTTGCTC |
| | CA |
| vt10-4 | CTGTGCGGCAACCTACATGATGGGTTTCAAACCCC |
| | GACCAGTTGGTAGGGGTTTACTTGCTGGCTTCCTC |
| | TAGCTTTTGTGGGTTTCAAACCCCGACCAGTTGGT |
| | AGGGGTTTACTTGTGGAGCAACAAATCTGACTTTG |
| | C |

Informal Sequence Listing
RfxCas13d Amino Acid Sequence:
(SEQ ID NO: 1)
MIEKKKSFAKGMGVKSTLVSGSKVYMTTFAEGSDARLEKIVEGDS
IRSVNEGEAFSAEMADKNAGYKIGNAKFSHPKGYAVVANNPLYTG
PVQQDMLGLKETLEKRYFGESADGNDNICIQVIHNILDIEKILAE
YITNAAYAVNNISGLDKDIIGFGKFSTVYTYDEFKDPEHHRAAFN
NNDKLINAIKAQYDEFDNFLDNPRLGYFGQAFFSKEGRNYIINYG
NECYDILALLSGLRHWVVHNNEEESRISRTWLYNLDKNLDNEYIS
TLNYLYDRITNELTNSFSKNSAANVNYIAETLGINPAEFAEQYFR
FSIMKEQKNLGFNITKLREVMLDRKDMSEIRKNHKVFDSIRTKVY
TMMDFVIYRYYIEEDAKVAAANKSLPDNEKSLSEKDIFVINLRGS
FNDDQKDALYYDEANRIWRKLENIMHNIKEFRGNKTREYKKKDAP
RLPRILPAGRDVSAFSKLMYALTMELDGKEINDLLTTLINKEDNI
QSFLKVMPLIGVNAKFVEEYAFFKDSAKIADELRLIKSFARMGEP
IADARRAMYIDAIRILGTNLSYDELKALADTFSLDENGNKLKKGK
HGMRNFIINNVISNKRFHYLIRYGDPAHLHEIAKNEAVVKFVLGR
IADIQKKQGQNGKNQIDRYYETCIGKDKGKSVSEKVDALTKIITG
MNYDQFDKKRSVIEDTGRENAEREKFKKIISLYLTVIYHILKNIV
NINARYVIGFHCVERDAQLYKEKGYDINLKKLEEKGFSSVTKLCA
GIDETAPDKRKDVEKEMAERAKESIDSLESANPKLYANYIKYSDE
KKAEEFTRQINREKAKTALNAYLRNTKWNVIIREDLLRIDNKTCT
LFRNKAVHLEVARYVHAYINDIAEVNSYFQLYHYIMQRIIMNERY
EKSSGKVSEYFDAVNDEKKYNDRLLKLLCVPFGYCIPRFKNLSIE
ALFDRNEAAKFDKEKKKVSGNS
> RfxCas13d-2XNLS-FLAG nucleic acid sequence
(SEQ ID NO: 2)
ATGATCGAGAAAAAGAAGTCCTTCGCTAAGGGCATGGGCGTGAAG
TCCACACTCGTGTCCGGCTCCAAAGTGTACATGACAACCTTCGCC
GAAGGCAGCGACGCCAGGCTGGAAAAGATCGTGGAGGGCGACAGC
ATCAGGAGCGTGAATGAGGGCGAGGCCTTCAGCGCTGAAATGGCC
GATAAAAACGCCGGCTATAAGATCGGCAACGCCAAATTCAGCCAT
CCTAAGGGCTACGCCGTGGTGGCTAACAACCCTCTGTATACAGGA
CCCGTCCAGCAGGATATGCTCGGCCTGAAGGAAACTCTGGAAAAG
AGGTACTTCGGCGAGAGCGCTGATGGCAATGACAATATTTGTATC
CAGGTGATCCATAACATCCTGGACATTGAAAAAATCCTCGCCGAA
TACATTACCAACGCCGCCTACGCCGTCAACAATATCTCCGGCCTG
GATAAGGACATTATTGGATTCGGCAAGTTCTCCACAGTGTATACC
TACGACGAATTCAAAGACCCCGAGCACCATAGGGCCGCTTTCAAC
AATAACGATAAGCTCATCAACGCCATCAAGGCCCAGTATGACGAG
TTCGACAACTTCCTCGATAACCCCAGACTCGGCTATTTCGGCCAG
GCCTTTTTCAGCAAGGAGGGCAGAAATTACATCATCAATTACGGC
AACGAATGCTATGACATTCTGGCCCTCCTGAGCGGACTGAGACAC
TGGGTGGTCCACAACAACGAAGAAGAGTCCAGGATCTCCAGGACC
TGGCTCTACAACCTCGATAAGAACCTCGACAACGAATACATCTCC
ACCCTCAACTACCTCTACGACAGGATCACCAATGAGCTGACCAAC
TCCTTCTCCAAGAACTCCGCCGCCAACGTGAACTATATTGCCGAA
ACTCTGGGAATCAACCCTGCCGAATTCGCCGAACAATATTTCAGA
TTCAGCATTATGAAAGAGCAGAAAAACCTCGGATTCAATATCACC
AAGCTCAGGGAAGTGATGCTGGACAGGAAGGATATGTCCGAGATC
AGGAAAAATCATAAGGTGTTCGACTCCATCAGGACCAAGGTCTAC
ACCATGATGGACTTTGTGATTTATAGGTATTACATCGAAGAGGAT
GCCAAGGTGGCTGCCGCCAATAAGTCCCTCCCCGATAATGAGAAG
TCCCTGAGCGAGAAGGATATCTTTGTGATTAACCTGAGGGGCTCC
TTCAACGACGACCAGAAGGATGCCCTCTACTACGATGAAGCTAAT
AGAATTTGGAGAAAGCTCGAAAATATCATGCACAACATCAAGGAA
TTTAGGGGAAACAAGACAAGAGAGTATAAGAAGAAGGACGCCCCT
AGACTGCCCAGAATCCTGCCCGCTGGCCGTGATGTTTCCGCCTTC
AGCAAACTCATGTATGCCCTGACAATGTTCCTGGATGGCAAGGAG
ATCAACGACCTCCTGACCACCCTGATTAATAAATTCGATAACATC
CAGAGCTTCCTGAAGGTGATGCCTCTCATCGGAGTCAACGCTAAG
TTCGTGGAGGAATACGCCTTTTTCAAAGACTCCGCCAAGATCGCC
GATGAGCTGAGGCTGATCAAGTCCTTCGCTAGAATGGGAGAACCT
ATTGCCGATGCCAGGAGGGCCATGTATATCGACGCCATCCGTATT
TTAGGAACCAACCTGTCCTATGATGAGCTCAAGGCCCTCGCCGAC
ACCTTTTCCCTGGACGAGAACGGAAACAAGCTCAAGAAAGGCAAG
CACGGCATGAGAAATTTCATTATTAATAACGTGATCAGCAATAAA
AGGTTCCACTACCTGATCAGATACGGTGATCCTGCCCACCTCCAT
GAGATCGCCAAAAACGAGGCCGTGGTGAAGTTCGTGCTCGGCAGG
ATCGCTGACATCCAGAAAAAACAGGGCCAGAACGGCAAGAACCAG
ATCGACAGGTACTACGAAACTTGTATCGGAAAGGATAAGGGCAAG
AGCGTGAGCGAAAAGGTGGACGCTCTCACAAAGATCATCACCGGA
ATGAACTACGACCAATTCGACAAGAAAAGGAGCGTCATTGAGGAC
ACCGGCAGGGAAAACGCCGAGAGGGAGAAGTTTAAAAAGATCATC
AGCCTGTACCTCACCGTGATCTACCACATCCTCAAGAATATTGTC
AATATCAACGCCAGGTACGTCATCGGATTCCATTGCGTCGAGCGT
GATGCTCAACTGTACAAGGAGAAAGGCTACGACATCAATCTCAAG
AAACTGGAAGAGAAGGGATTCAGCTCCGTCACCAAGCTCTGCGCT
GGCATTGATGAAACTGCCCCCGATAAGAGAAAGGACGTGGAAAAG
GAGATGGCTGAAAGAGCCAAGGAGAGCATTGACAGCCTCGAGAGC
GCCAACCCCAAGCTGTATGCCAATTACATCAAATACAGCGACGAG
AAGAAAGCCGAGGAGTTCACCAGGCAGATTAACAGGGAGAAGGCC
AAAACCGCCCTGAACGCCTACCTGAGGAACACCAAGTGGAATGTG
ATCATCAGGGAGGACCTCCTGAGAATTGACAACAAGACATGTACC
CTGTTCAGGAACAAGGCCGTCCATCTGGAAGTGGCCAGGTATGTC
CACGCCTATATCAACGACATTGCCGAGGTCAATTCCTACTTCCAA
CTGTACCATTACATCATGCAGAGAATTATCATGAATGAGAGGTAC
GAGAAAAGCAGCGGAAAGGTGTCCGAGTACTTCGACGCTGTGAAT
GACGAGAAGAAGTACAACGATAGGCTCCTGAAACTGCTGTGTGTG
CCTTTCGGCTACTGTATCCCCAGGTTTAAGAACCTGAGCATCGAG
GCCCTGTTCGATAGGAACGAGGCCGCCAAGTTCGACAAGGAGAAA
AAGAAAGTGTCCGGCAATTCCACTAGTGCTCCCAAGAAAAAGCGC
AAGGTAGGTGGAAGCCCAGCAGCTAAAAGAGTTAAATTGGATGGA
TCCGATTATAAGGATCACGATGGAGATTACAAGGACCACGACATA
GACTACAAAGATGATGACGACAAGTAA
> RfxCas13d-2XNLS-FLAG amino acid sequence
(SEQ ID NO: 3)
MIEKKKSFAKGMGVKSTLVSGSKVYMTTFAEGSDARLEKIVEGDS
IRSVNEGEAFSAEMADKNAGYKIGNAKFSHPKGYAVVANNPLYTG
PVQQDMLGLKETLEKRYFGESADGNDNICIQVIHNILDIEKILAE
YITNAAYAVNNISGLDKDIIGFGKFSTVYTYDEFKDPEHHRAAFN
NNDKLINAIKAQYDEFDNFLDNPRLGYFGQAFFSKEGRNYIINYG
NECYDILALLSGLRHWVVHNNEEESRISRTWLYNLDKNLDNEYIS
TLNYLYDRITNELTNSFSKNSAANVNVIAETLGINPAEFAEQYFR
FSIMKEQKNLGENITKLREVMLDRKDMSEIRKNHKVFDSIRTKVY
TMMDFVIYRYYIEEDAKVAAANKSLPDNEKSLSEKDIFVINLRGS
ENDDQKDALYYDEANRIWRKLENIMHNIKEFRGNKTREYKKKDAP
RLPRILPAGRDVSAFSKLMYALTMFLDGKEINDLLTTLINKEDNI
QSFLKVMPLIGVNAKFVEEYAFFKDSAKIADELRLIKSFARMGEP
IADARRAMYIDAIRILGTNLSYDELKALADTFSLDENGNKLKKGK
HGMRNFUNNVISNKRFHYLIRYGDPAHLHEIAKNEAVVKFVLGRI
ADIQKKQGQNGKNQIDRYVETCIGKDKGKSVSEKVDALTKIITGM
NYDQFDKKRSVIEDTGRENAEREKFKKIISLYLTVIVHILKNIVN
INARYVIGFHCVERDAQLYKEKGYDINLKKLEEKGFSSVTKLCAG
IDETAPDKRKDVEKEMAERAKESIDSLESANPKLYANYIKYSDEK
KAEEFTRQINREKAKTALNAYLRNTKWNVIIREDLLRIDNKTCTL
FRNKAVHLEVARYVHAYINDIAEVNSYFQLYHYIMQRIIMNERYE
KSSGKVSEYFDAVNDEKKYNDRLLKLLCVPFGYCIPRFKNLSIEA
LFDRNEAAKFDKEKKKVSGNSTSAPKKKRKVGGSPAAKRVKLDGS
DYKDHDGDYKDHDIDYKDDDDK
Bold: RfxCas13d
Underlined: 2XNLS
Italicized and underlined: FLAG tag
> DHFR DD domain nucleic acid sequence
(SEQ ID NO: 4)
ATATCACTGATCGCTGCACTCGCGGTTGATTATGTAATAGGCATG
GAAAACGCGATGCCCTGGAACCTGCCAGCAGACCTCGCATGGTTT
AAGCGGAACACTCTGAATAAGCCAGTCATTATGGGCCGACATACG
TGGGAAAGCATTGGACGCCCACTGCCAGGAAGGAAGAATATAATC
CTGTCATCACAGCCGTCCACGGACGATAGAGTCACTTGGGTAAAA
TCCGTTGACGAAGCCATAGCTGCCTGTGGTGATGTGCCTGAGATA
ATGGTAATAGGAGGAGGGCGGGTAATCGAACAATTTCTGCCTAAA
GCCCAGAAGCTGTACCTTACCCATATTGACGCGGAAGTAGAGGGC
GACACACATTTCCCAGATTATGAGCCAGATGATTGGGAGAGCGTT
TTCTCCGAATTTCATGATGCAGACGCCCAGAATAGCCACAGCTAC
TGCTTTGAAATATTGGAGCGCCGA
> DHFR DD domain amino acid sequence
(SEQ ID NO: 5)
ISLIAALAVDYVIGMENAMPWNLPADLAWFKRNTLNKPVIMGRHT
WESIGRPLPGRKNIILSSQPSTDDRVTWVKSVDEAIAACGDVPEI
MVIGGGRVIEQFLPKAQKLYLTHIDAEVEGDTHFPDYEPDDWESV
FSEFHDADAQNSHSYCFEILERR
> RfxCas13d-2XNLS-DD-FLAG nucleic acid sequence
(SEQ ID NO: 6)
ATGATCGAGAAAAAGAAGTCCTTCGCTAAGGGCATGGGCGTGAAG
TCCACACTCGTGTCCGGCTCCAAAGTGTACATGACAACCTTCGCC
GAAGGCAGCGACGCCAGGCTGGAAAAGATCGTGGAGGGCGACAGC
ATCAGGAGCGTGAATGAGGGCGAGGCCTTCAGCGCTGAAATGGCC
GATAAAAACGCCGGCTATAAGATCGGCAACGCCAAATTCAGCCAT
CCTAAGGGCTACGCCGTGGTGGCTAACAACCCTCTGTATACAGGA
CCCGTCCAGCAGGATATGCTCGGCCTGAAGGAAACTCTGGAAAAG
AGGTACTTCGGCGAGAGCGCTGATGGCAATGACAATATTTGTATC
CAGGTGATCCATAACATCCTGGACATTGAAAAAATCCTCGCCGAA
TACATTACCAACGCCGCCTACGCCGTCAACAATATCTCCGGCCTG
GATAAGGACATTATTGGATTCGGCAAGTTCTCCACAGTGTATACC
TACGACGAATTCAAAGACCCCGAGCACCATAGGGCCGCTTTCAAC
AATAACGATAAGCTCATCAACGCCATCAAGGCCCAGTATGACGAG
TTCGACAACTTCCTCGATAACCCCAGACTCGGCTATTTCGGCCAG
GCCTTTTTCAGCAAGGAGGGCAGAAATTACATCATCAATTACGGC
AACGAATGCTATGACATTCTGGCCCTCCTGAGCGGACTGAGACAC
TGGGTGGTCCACAACAACGAAGAAGAGTCCAGGATCTCCAGGACC
TGGCTCTACAACCTCGATAAGAACCTCGACAACGAATACATCTCC
ACCCTCAACTACCTCTACGACAGGATCACCAATGAGCTGACCAAC
TCCTTCTCCAAGAACTCCGCCGCCAACGTGAACTATATTGCCGAA
ACTCTGGGAATCAACCCTGCCGAATTCGCCGAACAATATTTCAGA
TTCAGCATTATGAAAGAGCAGAAAAACCTCGGATTCAATATCACC
AAGCTCAGGGAAGTGATGCTGGACAGGAAGGATATGTCCGAGATC
AGGAAAAATCATAAGGTGTTCGACTCCATCAGGACCAAGGTCTAC
ACCATGATGGACTTTGTGATTTATAGGTATTACATCGAAGAGGAT
GCCAAGGTGGCTGCCGCCAATAAGTCCCTCCCCGATAATGAGAAG
TOCCTGAGCGAGAAGGATATCTTTGTGATTAACCTGAGGGGCTCC
TTCAACGACGACCAGAAGGATGCCCTCTACTACGATGAAGCTAAT
AGAATTTGGAGAAAGCTCGAAAATATCATGCACAACATCAAGGAA
TTTAGGGGAAACAAGACAAGAGAGTATAAGAAGAAGGACGCCCCT
AGACTGCCCAGAATCCTGCCCGCTGGCCGTGATGTTTCCGCCTTC
AGCAAACTCATGTATGCCCTGACAATGTTCCTGGATGGCAAGGAG
ATCAACGACCTCCTGACCACCCTGATTAATAAATTCGATAACATC
CAGAGCTTCCTGAAGGTGATGCCTCTCATCGGAGTCAACGCTAAG
TTCGTGGAGGAATACGCCTTTTTCAAAGACTCCGCCAAGATCGCC
GATGAGCTGAGGCTGATCAAGTCCTTCGCTAGAATGGGAGAACCT
ATTGCCGATGCCAGGAGGGCCATGTATATCGACGCCATCCGTATT
TTAGGAACCAACCTGTCCTATGATGAGCTCAAGGCCCTCGCCGAC
ACCTTTTCCCTGGACGAGAACGGAAACAAGCTCAAGAAAGGCAAG
CACGGCATGAGAAATTTCATTATTAATAACGTGATCAGCAATAAA
AGGTTCCACTACCTGATCAGATACGGTGATCCTGCCCACCTCCAT
GAGATCGCCAAAAACGAGGCCGTGGTGAAGTTCGTGCTCGGCAGG
ATCGCTGACATCCAGAAAAAACAGGGCCAGAACGGCAAGAACCAG
ATCGACAGGTACTACGAAACTTGTATCGGAAAGGATAAGGGCAAG
AGCGTGAGCGAAAAGGTGGACGCTCTCACAAAGATCATCACCGGA
ATGAACTACGACCAATTCGACAAGAAAAGGAGCGTCATTGAGGAC
ACCGGCAGGGAAAACGCCGAGAGGGAGAAGTTTAAAAAGATCATC
AGCCTGTACCTCACCGTGATCTACCACATCCTCAAGAATATTGTC
AATATCAACGCCAGGTACGTCATCGGATTCCATTGCGTCGAGCGT
GATGCTCAACTGTACAAGGAGAAAGGCTACGACATCAATCTCAAG
AAACTGGAAGAGAAGGGATTCAGCTCCGTCACCAAGCTCTGCGCT
GGCATTGATGAAACTGCCCCCGATAAGAGAAAGGACGTGGAAAAG
GAGATGGCTGAAAGAGCCAAGGAGAGCATTGACAGCCTCGAGAGC
GCCAACCCCAAGCTGTATGCCAATTACATCAAATACAGCGACGAG
AAGAAAGCCGAGGAGTTCACCAGGCAGATTAACAGGGAGAAGGCC
AAAACCGCCCTGAACGCCTACCTGAGGAACACCAAGTGGAATGTG
ATCATCAGGGAGGACCTCCTGAGAATTGACAACAAGACATGTACC
CTGTTCAGGAACAAGGCCGTCCATCTGGAAGTGGCCAGGTATGTC
CACGCCTATATCAACGACATTGCCGAGGTCAATTCCTACTTCCAA
CTGTACCATTACATCATGCAGAGAATTATCATGAATGAGAGGTAC
GAGAAAAGCAGCGGAAAGGTGTCCGAGTACTTCGACGCTGTGAAT
GACGAGAAGAAGTACAACGATAGGCTCCTGAAACTGCTGTGTGTG
CCTTTCGGCTACTGTATCCCCAGGTTTAAGAACCTGAGCATCGAG
GCCCTGTTCGATAGGAACGAGGCCGCCAAGTTCGACAAGGAGAAA
AAGAAAGTGTCCGGCAATTCCACTAGTGCTCCCAAGAAAAAGCGC
AAGGTAGGTGGAAGCCCAGCAGCTAAAAGAGTTAAATTGGATGGA
TCCATATCACTGATCGCTGCACTCGCGGTTGATTATGTAATAGGC
ATGGAAAACGCGATGCCCTGGAACCTGCCAGCAGACCTCGCATGG
TTTAAGCGGAACACTCTGAATAAGCCAGTCATTATGGGCCGACAT
ACGTGGGAAAGCATTGGACGCCCACTGCCAGGAAGGAAGAATATA
ATCCTGTCATCACAGCCGTCCACGGACGATAGAGTCACTTGGGTA
AAATCCGTTGACGAAGCCATAGCTGCCTGTGGTGATGTGCCTGAG
ATAATGGTAATAGGAGGAGGGCGGGTAATCGAACAATTTCTGCCT
AAAGCCCAGAAGCTGTACCTTACCCATATTGACGCGGAAGTAGAG
GGCGACACACATTTCCCAGATTATGAGCCAGATGATTGGGAGAGC
GTTTTCTCCGAATTTCATGATGCAGACGCCCAGAATAGCCACAGC
TACTGCTTTGAAATATTGGAGCGCCGAGGATCCGATTATAAGGAT
CACGATGGAGATTACAAGGACCACGACATAGACTACAAAGATGAT
GACGACAAGTAA
> RfxCas13d-2XNLS-DD-FLAG amino acid sequence
(SEQ ID NO: 7)
MIEKKKSFAKGMGVKSTLVSGSKVYMTTFAEGSDARLEKIVEGDS
IRSVNEGEAFSAEMADKNAGYKIGNAKFSHPKGYAVVANNPLYTG
PVQQDMLGLKETLEKRYFGESADGNDNICIQVIHNILDIEKILAE
YITNAAYAVNNISGLDKDIIGFGKFSTVYTYDEFKDPEHHRAAFN
NNDKLINAIKAQYDEFDNFLDNPRLGYFGQAFFSKEGRNYIINYG
NECYDILALLSGLRHWVVHNNEEESRISRTWLYNLDKNLDNEYIS
TLNYLYDRITNELINSFSKNSAANVNYIAETLGINPAEFAEQYFR
FSIMKEQKNLGFNITKLREVMLDRKDMSEIRKNHKVFDSIRTKVY
TMMDFVIYRYYIEEDAKVAAANKSLPDNEKSLSEKDIFVINLRGS
FNDDQKDALYYDEANRIWRKLENIMHNIKEFRGNKTREYKKKDAP
RLPRILPAGRDVSAFSKLMYALIMFLDGKEINDLLTTLINKEDNI
QSFLKVMPLIGVNAKFVEEYAFFKDSAKIADELRLIKSFARMGEP
IADARRAMYIDAIRILGTNLSYDELKALADTFSLDENGNKLKKGK
HGMRNFIINNVISNKRFHYLIRYGDPAHLHEIAKNEAVVKFVLGR
IADIQKKQGQNGKNQIDRYYETCIGKDKGKSVSEKVDALTKITGM
NYDQFDKKRSVIEDTGRENAEREKFKKIISLYLTVIYHILKNIVN
INARYVIGFHCVERDAQLYKEKGYDINLKKLEEKGESSVTKLCAG
IDETAPDKRKDVEKEMAERAKESIDSLESANPKLYANYIKYSDEK
KAEEFTRQINREKAKTALNAYLRNTKWNVIIREDLLRIDNKTCTL
FRNKAVHLEVARYVHAYINDIAEVNSYFQLYHYIMQRIIMNERYE
KSSGKVSEYFDAVNDEKKYNDRLLKLLCVPFGYCIPRFKNLSIEA
LFDRNEAAKFDKEKKKVSGNSTSAPKKKRKVGGSPAAKRVKLDGS
ISLIAALAVDYVIGMENAMPWNLPADLAWFKRNTLNKPVIMGRHT
WESIGRPLPGRKNIILSSQPSTDDRVTWVKSVDEALAACGDVPEI
MVIGGGRVIEQFLPKAQKLYLTHIDAEVEGDTHFPDYEPDDWESV
FSEFHDADAQNSHSYCFEILERRGSDYKDHDGDYKDHDIDYKDDD
DK
Underlined: RfxCas13d
Bold: 2XNLS
Italicized: DHFR DD domain
Bold and underlined: FLAG tag
> RxCas13d example 10-plex guide array
nucleic acid sequence
(SEQ ID NO: 8)
AACCCCTACCAACTGGTCGGGGTTTGAAACGAGCAGTTCAAAATG
ACCCAGTCCAAGTAAACCCCTACCAACTGGTCGGGGTTTGAAACA
GCAACAGACGTAAGAACCAGAGCAAGTAAACCCCTACCAACTGGT
CGGGGTTTGAAACAAACCAAAGGAACATGACCAGCCCAAGTAAAC
CCCTACCAACTGGTCGGGGTTTGAAACATAGTTGATAGTAATCCA
GCCATCAAGTAAACCCCTACCAACTGGTCGGGGTTTGAAACAGAC
AATTGTGTCGCTGCCATCGCAAGTAAACCCCTACCAACTGGTCGG
GGTTTGAAACGCAAAGTCAGATTTGTTGCTCCACAAGTAAACCCC
TACCAACTGGTCGGGGTTTGAAACCCACAAAAGCTAGAGGAAGCC
AGCAAGTAAACCCCTACCAACTGGTCGGGGTTTGAAACCCATCAT
GTAGGTTGCCGCACAGCAAGTAAACCCCTACCAACTGGTCGGGGT
TTGAAACGGTACCAGTTTAGCACGAAGCTCCAAGTAAACCCCTAC
CAACTGGTCGGGGTTTGAAACCAGAAGAAAAGTCAGAGGACACC
Bold: DR30 (30 nucleotide direct repeat)
Underlined: DR36 (36 nucleotide direct repeat)
Direct Repeat Sequences:
(SEQ ID NO: 9)
AACCCCTACCAACTGGTCGGGGTTTGAAAC
(SEQ ID NO: 10)
CAAGTAAACCCCTACCAACTGGTCGGGGTTTGAAAC
Linker Sequence:
(SEQ ID NO: 11)
APKKKRKVGGSPAAKRVKLD

What is claimed is:

1. A genetically modified T cell comprising:
(i) a class 2 type VI-D CRISPR effector having RNA-guided RNA endonuclease activity; and
(ii) a guide array comprising a plurality of CRISPR-associated RNA (crRNA) molecules, wherein the crRNA molecules independently comprise a direct repeat sequence and a spacer sequence that binds a target RNA, and wherein the crRNA molecules bind to different target mRNAs or different regions of the same target mRNA.

2. The genetically modified T cell of claim 1, comprising a nucleic acid encoding (i), a nucleic acid encoding (ii), or a nucleic acid encoding both (i) and (ii), wherein the nucleic acids encoding (i) or (ii), or both (i) and (ii), are stably integrated into the genome of the T cell.

3. The genetically modified T cell of claim 1 or 2, wherein the class 2 type VI-D CRISPR effector having RNA-guided RNA endonuclease activity comprises a fusion protein comprising a destabilization domain (DD).

4. The genetically modified T cell of any one of claims 1 to 3, wherein the class 2 type VI-D CRISPR effector having RNA-guided RNA endonuclease activity is a Cas13 nuclease selected from the group consisting of Cas13a, Cas13b, 13bt1, Cas13bt2, Cas13c, Cas13d, RfxCas13d, Cas13e, and functional variants thereof.

5. The genetically modified T cell of any one of claims 1 to 4, wherein the guide array is a multicistronic array comprising the plurality of crRNA molecules.

6. The genetically modified T cell of any one of claims 1 to 5, wherein the guide array comprises from 2 to 10 crRNA molecules.

7. The genetically modified T cell of any one of claims 1 to 5, wherein the guide array comprises greater than 10 crRNA molecules.

8. The genetically modified T cell of any one of claims 1 to 7, wherein the guide array comprises crRNA molecules that bind to an mRNA encoding a protein associated with T cell exhaustion.

9. The genetically modified T cell of claim 8, wherein the protein associated with T cell exhaustion is selected from the group consisting of TOX, TOX2, NR4A1, NR4A2, NR4A3, TET2, IRF4, JUNB, BATF3, DHX37, FLI1, ZC3H12A, SOCS1, TCEB2, PDCD1 (PD-1), HAVCR2 (TIM3), LAG3, CTLA4, TIGIT, FAS, TRAC, CBLB, RASA2, PTPN2, CD5, ENTPD1 (CD39), CD46, B2M, ZAP70, LCK, AKT1, AKT2, HK1, HK2, NT5E (CD73), ADORA2A, ADORA2B, LDHA, LDHB, CD147, MCT1, MCT4, GAPDH, and combinations thereof.

10. The genetically modified T cell of any one of claims 1 to 9, wherein the T cell further comprises a chimeric antigen receptor (CAR).

11. The genetically modified T cell of claim 10, wherein the CAR binds to an antigen expressed by a tumor.

12. The genetically modified T cell of claim 10 or 11, wherein the CAR binds to an antigen selected from the group consisting of Her-2, B7-H3, GPC2, GD2, CD19, CD20, CD22, MAGE, BAGE, CAGE, GAGE, HAGE, LAGE, PAGE, PRAME, NY-ESO-1, NY-SEO-1, tyrosinase, Melan-A/MART, gp100, TRP-1, TRP-2, CD30, EGFR, EGFRvIII, FAP, CD33, CD123, PD-L1, IGF1R, CD4, CSPG4, B7-H4, NKG2D, CS1, CD138, EpCAM, EBNA3C, GPA7, CD244, CA-125, ETA, CEA, CD52, MUCSAC, c-Met, FAB, WT-1, PSMA, AFP, BCMA, Mesothelin, GPC3, MUC1 and CTAG1B.

13. The genetically modified T cell of any one of claims 10 to 12, wherein intracellular signaling by the CAR upregulates T cell exhaustion markers in control T cells that do not comprise (i) and (ii) of claim 1.

14. The genetically modified T cell of claim 13, wherein the exhaustion markers are selected from the group consisting of LAG3, PDCD1 (PD-1), HAVCR2 (TIM3), CTLA4, ENTPD1, and combinations thereof.

15. The genetically modified T cell of any one of claims 10 to 14, wherein the guide array comprises crRNA molecules that bind to mRNA encoding LAG3, PDCD1 (PD-1), HAVCR2 (TIM3), CTLA4, ENTPD1, or a combination thereof.

16. The genetically modified T cell of any one of claims 10 to 15, wherein the guide array comprises one or more crRNA molecules that bind to mRNA expressed by the CAR.

17. The genetically modified T cell of any one of claims 1 to 16, wherein the T cell is selected from the group consisting of a human T cell and a primary human T cell.

18. A nucleic acid encoding:
(i) a class 2 type VI-D CRISPR effector having RNA-guided RNA endonuclease activity; and
(ii) a guide array comprising a plurality of CRISPR-associated RNA (crRNA) molecules, wherein the crRNA molecules independently comprise a direct repeat sequence and a spacer sequence that binds a target RNA, and wherein the crRNA molecules bind to different target mRNAs or different regions of the same target mRNA.

19. The nucleic acid of claim 18, wherein the class 2 type VI-D CRISPR effector having RNA-guided RNA endonuclease activity is a Cas13 nuclease selected from the group consisting of Cas13a, Cas13b, 13bt1, Cas13bt2, Cas13c, Cas13d, RfxCas13d, Cas13e, and functional variants thereof.

20. The nucleic acid of claim 18 or 19, wherein the guide array is a multicistronic array comprising the plurality of crRNA molecules.

21. The nucleic acid of any one of claims 18 to 20, wherein the guide array comprises from 2 to greater than or equal to 10 crRNA molecules.

22. The nucleic acid of any one of claims 18 to 21, wherein the guide array comprises crRNA molecules that bind to mRNA encoding a protein associated with T cell exhaustion.

23. The nucleic acid of claim 22, wherein the protein associated with T cell exhaustion is selected from the group consisting of TOX, TOX2, NR4A1, NR4A2, NR4A3, TET2, IRF4, JUNB, BATF3, DHX37, FLI1, ZC3H12A, SOCS1, TCEB2, PDCD1 (PD-1), HAVCR2 (TIM3), LAG3, CTLA4, TIGIT, FAS, TRAC, CBLB, RASA2, PTPN2, CDS, ENTPD1 (CD39), CD46, B2M, ZAP70, LCK, AKT1, AKT2, HK1, HK2, NTSE (CD73), ADORA2A, ADORA2B, LDHA, LDHB, CD147, MCT1, MCT4, GAPDH, and combinations thereof.

24. A system for multiplexed transcriptomic regulation, comprising:
(i) an expression cassette comprising a nucleic acid sequence encoding a class 2 type VI-D CRISPR effector having RNA-guided RNA endonuclease activity; and
(ii) an expression cassette comprising a nucleic acid sequence encoding a guide array comprising a plurality of crRNA molecules, wherein the crRNA molecules comprise a direct repeat sequence and a spacer sequence that binds a target mRNA, and wherein the crRNA molecules bind to different target mRNAs or different regions of the same target mRNA.

25. The system of claim 24, wherein the class 2 type VI-D CRISPR effector having RNA-guided RNA endonuclease activity is a Cas13 nuclease selected from the group consisting of Cas13a, Cas13b, 13bt1, Cas13bt2, Cas13c, Cas13d, RfxCas13d, Cas13e, and functional variants thereof.

26. The system of claim 24 or 25, wherein the guide array is a multicistronic array comprising the plurality of crRNA molecules.

27. The system of any one of claims 24 to 26, wherein the guide array comprises from 2 to greater than or equal to 10 crRNA molecules.

28. The system of any one of claims 24 to 27, wherein the guide array comprises crRNA molecules that bind to mRNA encoding a protein associated with T cell exhaustion.

29. The system of claim 28, wherein the protein associated with T cell exhaustion is selected from the group consisting of TOX, TOX2, NR4A1, NR4A2, NR4A3, TET2, IRF4, JUNB, BATF3, DHX37, FLI1, ZC3H12A, SOCS1, TCEB2, PDCD1 (PD-1), HAVCR2 (TIM3), LAG3, CTLA4, TIGIT, FAS, TRAC, CBLB, RASA2, PTPN2, CDS, ENTPD1 (CD39), CD46, B2M, ZAP70, LCK, AKT1, AKT2, HK1, HK2, NTSE (CD73), ADORA2A, ADORA2B, LDHA, LDHB, CD147, MCT1, MCT4, GAPDH, and combinations thereof.

30. The system of any one of claims 24 to 29, wherein the system further comprises (iii) a T cell or primary T cell.

31. The system of claim 30, wherein the T cell or primary T cell further comprises a chimeric antigen receptor (CAR).

32. The system of claim 31, wherein the CAR binds to an antigen expressed by a tumor.

33. The system of claim 31 or 32, wherein the CAR binds to an antigen selected from the group consisting of Her-2, B7-H3, GPC2, GD2, CD19, CD20, CD22, MAGE, BAGE, CAGE, GAGE, HAGE, LAGE, PAGE, PRAME, NY-ESO-1, NY-SEO-1, tyrosinase, Melan-A/MART, gpl00, TRP-1, TRP-2, CD30, EGFR, EGFRvIII, FAP, CD33, CD123, PD-L1, IGF1R, CD4, CSPG4, B7-H4, NKG2D, CSI, CD138, EpCAM, EBNA3C, GPA7, CD244, CA-125, ETA, CEA, CD52, MUC5AC, c-Met, FAB, WT-1, PSMA, AFP, BCMA, Mesothelin, GPC3, MUC1 and CTAG1B.

34. The system of any one of claims 31 to 33, wherein expression and intracellular signaling by the CAR upregulates T cell exhaustion markers in control T cells that do not comprise (i) and (ii) of claim 24.

35. The system of claim 34, wherein the exhaustion markers are selected from LAG3, PDCD1 (PD-1), HAVCR2 (TIM3), CTLA4, ENTPD1, or a combination thereof.

36. The system of any one of claims 31 to 35, wherein the guide array comprises crRNA molecules that bind to mRNA encoding LAG3, PDCD1 (PD-1), HAVCR2 (TIM3), CTLA4, ENTPD1, or a combination thereof.

37. The system of any one of claims 31 to 36, wherein the guide array comprises one or more crRNA molecules that bind to mRNA expressed by the CAR.

38. The system of any one of claims 30 to 37, wherein the T cell is selected from the group consisting of a human T cell and a primary human T cell.

39. A method for producing a modified T cell, comprising:
(i) transducing a T cell with an expression vector comprising a nucleic acid sequence encoding a class 2 type VI-D CRISPR effector having RNA-guided RNA endonuclease activity; and
(ii) transducing the T cell with an expression vector comprising a nucleic acid sequence encoding a guide array comprising a plurality of CRISPR-associated RNA (crRNA) molecules, wherein the crRNA molecules independently comprise a direct repeat sequence and a spacer sequence that binds a target mRNA, and wherein the crRNA molecules bind to different target mRNAs or different regions of the same target mRNA,
thereby producing the modified T cell.

40. The method of claim 39, further comprising (iii) transducing the T cell with an expression vector comprising a nucleic acid sequence encoding a CAR.

41. The method of claim 39 or 40, wherein step (i) is performed before step (ii); step (iii) is performed before step (ii), or steps (i) and (iii) are performed before step (ii).

42. The method of any one of claims 39 to 41, wherein (i), (ii) and/or (iii) are stably integrated into the genome of the T cell.

43. The method of any one of claims 39 to 42, wherein the class 2 type VI-D CRISPR effector having RNA-guided RNA endonuclease activity is a Cas13 nuclease selected from the group consisting of Cas13a, Cas13b, 13bt1, Cas13bt2, Cas13c, Cas13d, RfxCas13d, Cas13e, and functional variants thereof.

44. The method of any one of claims 39 to 43, wherein the guide array is a multicistronic array comprising the plurality of crRNA molecules.

45. The method of any one of claims 39 to 44, wherein the guide array comprises from 2 to greater than or equal to 10 crRNA molecules.

46. The method of any one of claims 39 to 45, wherein the guide array comprises crRNA molecules that bind to an mRNA encoding a protein associated with T cell exhaustion.

47. The method of claim 46, wherein the protein associated with T cell exhaustion is selected from the group consisting of TOX, TOX2, NR4A1, NR4A2, NR4A3, TET2, IRF4, JUNB, BATF3, DHX37, FLI1, ZC3H12A, SOCS1, TCEB2, PDCD1 (PD-1), HAVCR2 (TIM3), LAG3, CTLA4, TIGIT, FAS, TRAC, CBLB, RASA2, PTPN2, CD5, ENTPD1 (CD39), CD46, B2M, ZAP70, LCK, AKT1, AKT2, HK1, HK2, NTSE (CD73), ADORA2A, ADORA2B, LDHA, LDHB, CD147, MCT1, MCT4, GAPDH, and combinations thereof.

48. The method of any one of claims 40 to 47, wherein the CAR binds to an antigen expressed by a tumor.

49. The method of any one of claims 40 to 48, wherein the CAR binds to an antigen selected from the group consisting of Her-2, B7-H3, GPC2, GD2, CD19, CD20, CD22, MAGE, BAGE, CAGE, GAGE, HAGE, LAGE, PAGE, PRAME, NY-ESO-1, NY-SEO-1, tyrosinase, Melan-A/MART, gpl00, TRP-1, TRP-2, CD30, EGFR, EGFRvIII, FAP, CD33, CD123, PD-L1, IGF1R, CD4, CSPG4, B7-H4, NKG2D, CSI, CD138, EpCAM, EBNA3C, GPA7, CD244, CA-125, ETA, CEA, CD52, MUCSAC, c-Met, FAB, WT-1, PSMA, AFP, BCMA, Mesothelin, GPC3, MUC1 and CTAG1B.

50. The method of any one of claims 40 to 49, wherein expression and intracellular signaling by the CAR upregulates T cell exhaustion markers in control T cells that do not comprise the expression vectors of steps (i) and (ii).

51. The method of claim 50, wherein the exhaustion markers are selected from LAG3, PDCD1 (PD-1), or HAVCR2 (TIM3), or a combination thereof.

52. The method of any one of claims 40 to 51, wherein the guide array comprises crRNA molecules that bind to mRNA expressed by the CAR.

53. The method of any one of claims 39 to 52, wherein the T cell is selected from the group consisting of a human T cell and a primary human T cell.

54. A fusion protein comprising a class 2 type VI-D CRISPR effector having RNA-guided RNA endonuclease activity covalently linked to a destabilization domain (DD) polypeptide.

55. The fusion protein of claim 54, wherein the class 2 type VI-D CRISPR effector having RNA-guided RNA endonuclease activity is a Cas13 nuclease selected from the group consisting of Cas13a, Cas13b, 13bt1, Cas13bt2, Cas13c, Cas13d, RfxCas13d, Cas13e, and functional variants thereof.

56. The fusion protein of claim 54 or 55, wherein the DD comprises an E. coli dihydrofolate reductase DD linked to the C-terminus of the class 2 type VI-D CRISPR effector having RNA-guided RNA endonuclease activity.

57. A nucleic acid encoding the fusion protein of any one of claims 54 to 56.

58. A method for regulating gene expression in a T cell, comprising:
(a) transducing a T cell with
(i) an expression vector comprising a nucleic acid sequence encoding a fusion protein of any one of claims 54 to 56; and
(ii) an expression vector comprising a nucleic acid sequence encoding a guide array comprising a crRNA molecule, wherein the crRNA molecule comprises a direct repeat sequence and a spacer sequence that binds a target mRNA expressed by a target gene,
(b) contacting the T cell with a compound that binds to and stabilizes the DD, wherein expression of the target gene is decreased in the presence of the compound compared to expression of the target gene in the absence of the compound,
thereby regulating gene expression in the T cell.

59. The method of claim 58, wherein the fusion protein is degraded in the T cell in the absence of the compound.

60. The method of claim 58 or 59, wherein expression of the target gene is increased after removal of the compound, thereby reversibly regulating expression of the target gene.

61. The method of any one of claims 58 to 60, wherein the guide array comprises a plurality of crRNA molecules that bind to different target mRNAs or different regions of the same target mRNA.

62. The method of any one of claims 58 to 61, wherein expression of the target gene(s) is regulated by the compound in a dose-dependent manner.

63. The method of any one of claims 58 to 62, wherein the compound is trimethoprim (TMP).

64. A method for screening to identify regulators of T cell activity, comprising expressing in a T cell or population of T cells:
(i) a class 2 type VI-D CRISPR effector having RNA-guided RNA endonuclease activity; and
(ii) a library of guide arrays, where individual guide arrays comprise one or more CRISPR-associated RNA (crRNA) molecules, wherein the crRNA molecules comprise a direct repeat sequence and a spacer sequence that binds a target RNA, and wherein the one or more crRNA molecules bind to different target mRNAs or different regions of the same target mRNA;
culturing the T cell(s) to produce a clonal population of expanded T cells; and
determining if a guide array is enriched or depleted in the clonal population of expanded T cells,
wherein if a guide array is enriched, then the target mRNA encodes a negative regulator of T cell activity, or if a guide array is depleted, then the target mRNA encodes a positive regulator of T cell activity.

65. The method of claim 64, wherein the T cell activity is T cell proliferation, increased cytokine secretion, or increased tumor cell killing.

66. The method of claim 64 or 65, wherein T cells comprising an enriched guide array have an effector memory phenotype, and T cells comprising a depleted guide array have a central memory or stem cell memory phenotype.

67. The method of any one of claims 64 to 66, wherein the individual guide arrays comprise crRNA molecules that bind to an mRNA encoding a protein associated with T cell exhaustion.

68. The method of claim 67, wherein the protein associated with T cell exhaustion is selected from the group consisting of TOX, TOX2, NR4A1, NR4A2, NR4A3, TET2, IRF4, JUNB, BATF3, DHX37, FLI1, ZC3H12A, SOCS1, TCEB2, PDCD1 (PD-1), HAVCR2 (TIM3), LAG3, CTLA4, TIGIT, FAS, TRAC, CBLB, RASA2, PTPN2, CD5, ENTPD1 (CD39), CD46, B2M, ZAP70, LCK, AKT1, AKT2, HK1, HK2, NT5E (CD73), ADORA2A, ADORA2B, LDHA, LDHB, CD147, MCT1, MCT4, GAPDH, and combinations thereof.

69. The method of any one of claims 64 to 68, wherein the library of guide arrays comprises one or more individual guide arrays comprising multicistronie arrays comprising a plurality of crRNA molecules.

70. The method of any one of claims 64 to 69, wherein the individual guide arrays comprise from 2 to 10 crRNA molecules.

71. The method of any one of claims 64 to 70, wherein the individual guide arrays comprise a pair of crRNA molecules.

72. The method of any one of claims 64 to 71, wherein the class 2 type VI-D CRISPR effector having RNA-guided RNA endonuclease activity is a Cas13 nuclease selected from the group consisting of Cas13a, Cas13b, 13bt1, Cas13bt2, Cas13c, Cas13d, RfxCas13d, Cas13e, and functional variants thereof.

73. The method of any one of claims 64 to 72, wherein the T cell further expresses a chimeric antigen receptor (CAR).

74. The method of claim 73, wherein the CAR binds to an antigen expressed by a tumor.

75. The method of claim 73 or 74, wherein the CAR binds to an antigen selected from the group consisting of Her-2, B7-H3, GPC2, GD2, CD19, CD20, CD22, MAGE, BAGE, CAGE, GAGE, HAGE, LAGE, PAGE, PRAME, NY-ESO-1, NY-SEO-1, tyrosinase, Melan-A/MART, gp100, TRP-1, TRP-2, CD30, EGFR, EGFRvIII, FAP, CD33, CD123, PD-L1, IGFIR, CD4, CSPG4, B7-H4, NKG2D, CSI, CD138, EpCAM, EBNA3C, GPA7, CD244, CA-125, ETA, CEA, CD52, MUCSAC, c-Met, FAB, WT-1, PSMA, AFP, BCMA, Mesothelin, GPC3, MUC1 and CTAG1B.

76. The method of any one of claims 64 to 75, wherein determining if a guide array is enriched or depleted in the clonal population of expanded T cells comprises sequencing the guide RNAs present in the T cells.

77. A method for increasing proliferation of a T cell, the method comprising transducing a T cell with
(i) an expression vector comprising a nucleic acid sequence encoding a class 2 type VI-D CRISPR effector having RNA-guided RNA endonuclease activity; and
(ii) an expression vector comprising a nucleic acid sequence encoding a guide array comprising one or more crRNA molecules, wherein the crRNA molecules independently comprise a direct repeat sequence and a spacer sequence that binds a target mRNA, and wherein the crRNA molecules bind to different target mRNAs or different regions of the same target mRNA,
wherein proliferation of the T cell is increased compared to a control T cell that expresses a non-targeting control guide array comprising one or more crRNA molecules that do not bind to a target mRNA in (ii).

78. The method of claim 77, wherein the guide array comprises crRNA molecules that bind to an mRNA encoding a protein associated with T cell exhaustion.

79. The method of claim 78, wherein the protein associated with T cell exhaustion is selected from the group consisting of TOX, TOX2, NR4A1, NR4A2, NR4A3, TET2, IRF4, JUNB, BATF3, DHX37, FLI1, ZC3H12A, SOCS1, TCEB2, PDCD1, HAVCR2, LAG3, CTLA4, TIGIT, FAS, TRAC, CBLB, RASA2, PTPN2, CD5, ENTPD1 (CD39), CD46, B2M, ZAP70, LCK, AKT1, AKT2, HK1, HK2, NT5E (CD73), ADORA2A, ADORA2B, LDHA, LDHB, CD147, MCT1, MCT4, GAPDH, and combinations thereof.

80. The method of any one of claims 77 to 79, wherein the guide array comprises a multicistronic array comprising a plurality of crRNA molecules.

81. The method of any one of claims 77 to 80, wherein the guide array comprises from 2 to greater than or equal to 10 crRNA molecules.

82. The method of any one of claims 77 to 81, wherein the class 2 type VI-D CRISPR effector having RNA-guided RNA endonuclease activity is a Cas13 nuclease selected from the group consisting of Cas13a, Cas13b, 13bt1, Cas13bt2, Cas13c, Cas13d, RfxCas13d, Cas13e, and functional variants thereof.

83. The method of any one of claims 77 to 82, further comprising (iii) transducing the T cell with an expression vector comprising a nucleic acid sequence encoding a CAR.

84. The method of claim 83, wherein the CAR binds to an antigen expressed by a tumor.

85. The method of claim 83 or 84, wherein the CAR binds to an antigen selected from the group consisting of Her-2, B7-H3, GPC2, GD2, CD19, CD20, CD22, MAGE, BAGE, CAGE, GAGE, HAGE, LAGE, PAGE, PRAME, NY-ESO-1, NY-SEO-1, tyrosinase, Melan-A/MART, gp100, TRP-1, TRP-2, CD30, EGFR, EGFRvIII, FAP, CD33, CD123, PD-L1, IGF1R, CD4, CSPG4, B7-H4, NKG2D, CS1, CD138, EpCAM, EBNA3C, GPA7, CD244, CA-125, ETA, CEA, CD52, MUC5AC, c-Met, FAB, WT-1, PSMA, AFP, BCMA, Mesothelin, GPC3, MUC1 and CTAG1B.

86. A method for treating a tumor in a subject, comprising:
administering a genetically modified T cell of any one of claims 1 to 17 to the subject, wherein the modified T cell kills tumor cells in the subject, thereby treating the tumor.

87. A method for increasing the anti-tumor activity of a T cell, the method comprising contacting a tumor cell with a genetically modified T cell of any one of claims 1 to 17, wherein contacting the tumor cell with the genetically modified T cell increases the expression of anti-tumor cytokines or kills the tumor cell, thereby increasing the anti-tumor activity compared to a control T cell that does not comprise (i) or (ii) or both (i) and (ii).

88. The method of claim 87, wherein the method is an in vitro method.

89. The method of claim 87, wherein the method is an in vivo method.

90. A guide array comprising a plurality of CRISPR-associated RNA (crRNA) molecules, wherein the crRNA molecules comprise a direct repeat sequence and a spacer sequence that binds a target RNA, and wherein the crRNA molecules bind to different target mRNAs or different regions of the same target mRNA.

91. The guide array of claim 90, wherein the guide array is a multicistronic array comprising the plurality of crRNA molecules.

92. The guide array of claim 90 or 91, wherein the guide array comprises from 2 to 10 crRNA molecules.

93. The guide array of claim 90 or 91, wherein the guide array comprises greater than 10 crRNA molecules.

94. The guide array of any one of claims 90 to 93, wherein the guide array comprises crRNA molecules that bind to an mRNA encoding a protein associated with T cell exhaustion.

95. The guide array of claim 94, wherein the protein associated with T cell exhaustion is selected from the group consisting of TOX, TOX2, NR4A1, NR4A2, NR4A3, TET2, IRF4, JUNB, BATF3, DHX37, FLI1, ZC3H12A, SOCS1, TCEB2, PDCD1 (PD-1), HAVCR2 (TIM3), LAG3, CTLA4, TIGIT, FAS, TRAC, CBLB, RASA2, PTPN2, CD5, ENTPD1 (CD39), CD46, B2M, ZAP70, LCK, AKT1, AKT2, HK1, HK2, NT5E (CD73), ADORA2A, ADORA2B, LDHA, LDHB, CD147, MCT1, MCT4, GAPDH, and combinations thereof.


SEQUENCE LISTING (109 sequences)

SEQ ID NO: 1 - protein, 967 aa, synthetic construct
MIEKKKSFAKGMGVKSTLVSGSKVYMTTFAEGSDARLEKIVEGDSIRSVNEGEAFSAEMADKNAGYKIGNAKFSHPKGYAVVANNPLYTGPVQQDMLGLKETLEKRYFGESADGNDNICIQVIHNILDIEKILAEYITNAAYAVNNISGLDKDIIGFGKFSTVYTYDEFKDPEHHRAAFNNNDKLINAIKAQYDEFDNFLDNPRLGYFGQAFFSKEGRNYIINYGNECYDILALLSGLRHWVVHNNEEESRISRTWLYNLDKNLDNEYISTLNYLYDRITNELTNSFSKNSAANVNYIAETLGINPAEFAEQYFRFSIMKEQKNLGFNITKLREVMLDRKDMSEIRKNHKVFDSIRTKVYTMMDFVIYRYYIEEDAKVAAANKSLPDNEKSLSEKDIFVINLRGSFNDDQKDALYYDEANRIWRKLENIMHNIKEFRGNKTREYKKKDAPRLPRILPAGRDVSAFSKLMYALTMFLDGKEINDLLTTLINKFDNIQSFLKVMPLIGVNAKFVEEYAFFKDSAKIADELRLIKSFARMGEPIADARRAMYIDAIRILGTNLSYDELKALADTFSLDENGNKLKKGKHGMRNFIINNVISNKRFHYLIRYGDPAHLHEIAKNEAVVKFVLGRIADIQKKQGQNGKNQIDRYYETCIGKDKGKSVSEKVDALTKIITGMNYDQFDKKRSVIEDTGRENAEREKFKKIISLYLTVIYHILKNIVNINARYVIGFHCVERDAQLYKEKGYDINLKKLEEKGFSSVTKLCAGIDETAPDKRKDVEKEMAERAKESIDSLESANPKLYANYIKYSDEKKAEEFTRQINREKAKTALNAYLRNTKWNVIIREDLLRIDNKTCTLFRNKAVHLEVARYVHAYINDIAEVNSYFQLYHYIMQRIIMNERYEKSSGKVSEYFDAVNDEKKYNDRLLKLLCVPFGYCIPRFKNLSIEALFDRNEAAKFDKEKKKVSGNS

SEQ ID NO: 2 - DNA, 3042 bp, synthetic construct
  source 1..3042: mol_type = other DNA
atgatcgagaaaaagaagtccttcgctaagggcatgggcgtgaagtccacactcgtgtccggctccaaagtgtacatgacaaccttcgccgaaggcagcgacgccaggctggaaaagatcgtggagggcgacagcatcaggagcgtgaatgagggcgaggccttcagcgctgaaatggccgataaaaacgccggctataagatcggcaacgccaaattcagccatcctaagggctacgccgtggtggctaacaaccctctgtatacaggacccgtccagcaggatatgctcggcctgaaggaaactctggaaaagaggtacttcggcgagagcgctgatggcaatgacaatatttgtatccaggtgatccataacatcctggacattgaaaaaatcctcgccgaatacattaccaacgccgcctacgccgtcaacaatatctccggcctggataaggacattattggattcggcaagttctccacagtgtatacctacgacgaattcaaagaccccgagcaccatagggccgctttcaacaataacgataagctcatcaacgccatcaaggcccagtatgacgagttcgacaacttcctcgataaccccagactcggctatttcggccaggcctttttcagcaaggagggcagaaattacatcatcaattacggcaacgaatgctatgacattctggccctcctgagcggactgagacactgggtggtccacaacaacgaagaagagtccaggatctccaggacctggctctacaacctcgataagaacctcgacaacgaatacatctccaccctcaactacctctacgacaggatcaccaatgagctgaccaactccttctccaagaactccgccgccaacgtgaactatattgccgaaactctgggaatcaaccctgccgaattcgccgaacaatatttcagattcagcattatgaaagagcagaaaaacctcggattcaatatcaccaagctcagggaagtgatgctggacaggaaggatatgtccgagatcaggaaaaatcataaggtgttcgactccatcaggaccaaggtctacaccatgatggactttgtgatttataggtattacatcgaagaggatgccaaggtggctgccgccaataagtccctccccgataatgagaagtccctgagcgagaaggatatctttgtgattaacctgaggggctccttcaacgacgaccagaaggatgccctctactacgatgaagctaatagaatttggagaaagctcgaaaatatcatgcacaacatcaaggaatttaggggaaacaagacaagagagtataagaagaaggacgcccctagactgcccagaatcctgcccgctggccgtgatgtttccgccttcagcaaactcatgtatgccctgacaatgttcctggatggcaaggagatcaacgacctcctgaccaccctgattaataaattcgataacatccagagcttcctgaaggtgatgcctctcatcggagtcaacgctaagttcgtggaggaatacgcctttttcaaagactccgccaagatcgccgatgagctgaggctgatcaagtccttcgctagaatgggagaacctattgccgatgccaggagggccatgtatatcgacgccatccgtattttaggaaccaacctgtcctatgatgagctcaaggccctcgccgacaccttttccctggacgagaacggaaacaagctcaagaaaggcaagcacggcatgagaaatttcattattaataacgtgatcagcaataaaaggttccactacctgatcagatacggtgatcctgcccacctccatgagatcgccaaaaacgaggccgtggtgaagttcgtgctcggcaggatcgctgacatccagaaaaaacagggccagaacggcaagaaccagatcgacaggtactacgaaacttgtatcggaaaggataagggcaagagcgtgagcgaaaaggtggacgctctcacaaagatcatcaccggaatgaactacgaccaattcgacaagaaaaggagcgtcattgaggacaccggcagggaaaacgccgagagggagaagtttaaaaagatcatcagcctgtacctcaccgtgatctaccacatcctcaagaatattgtcaatatcaacgccaggtacgtcatcggattccattgcgtcgagcgtgatgctcaactgtacaaggagaaaggctacgacatcaatctcaagaaactggaagagaagggattcagctccgtcaccaagctctgcgctggcattgatgaaactgcccccgataagagaaaggacgtggaaaaggagatggctgaaagagccaaggagagcattgacagcctcgagagcgccaaccccaagctgtatgccaattacatcaaatacagcgacgagaagaaagccgaggagttcaccaggcagattaacagggagaaggccaaaaccgccctgaacgcctacctgaggaacaccaagtggaatgtgatcatcagggaggacctcctgagaattgacaacaagacatgtaccctgttcaggaacaaggccgtccatctggaagtggccaggtatgtccacgcctatatcaacgacattgccgaggtcaattcctacttccaactgtaccattacatcatgcagagaattatcatgaatgagaggtacgagaaaagcagcggaaaggtgtccgagtacttcgacgctgtgaatgacgagaagaagtacaacgataggctcctgaaactgctgtgtgtgcctttcggctactgtatccccaggtttaagaacctgagcatcgaggccctgttcgataggaacgaggccgccaagttcgacaaggagaaaaagaaagtgtccggcaattccactagtgctcccaagaaaaagcgcaaggtaggtggaagcccagcagctaaaagagttaaattggatggatccgattataaggatcacgatggagattacaaggaccacgacatagactacaaagatgatgacgacaagtaa

SEQ ID NO: 3 - protein, 1013 aa, synthetic construct
MIEKKKSFAKGMGVKSTLVSGSKVYMTTFAEGSDARLEKIVEGDSIRSVNEGEAFSAEMADKNAGYKIGNAKFSHPKGYAVVANNPLYTGPVQQDMLGLKETLEKRYFGESADGNDNICIQVIHNILDIEKILAEYITNAAYAVNNISGLDKDIIGFGKFSTVYTYDEFKDPEHHRAAFNNNDKLINAIKAQYDEFDNFLDNPRLGYFGQAFFSKEGRNYIINYGNECYDILALLSGLRHWVVHNNEEESRISRTWLYNLDKNLDNEYISTLNYLYDRITNELTNSFSKNSAANVNYIAETLGINPAEFAEQYFRFSIMKEQKNLGFNITKLREVMLDRKDMSEIRKNHKVFDSIRTKVYTMMDFVIYRYYIEEDAKVAAANKSLPDNEKSLSEKDIFVINLRGSFNDDQKDALYYDEANRIWRKLENIMHNIKEFRGNKTREYKKKDAPRLPRILPAGRDVSAFSKLMYALTMFLDGKEINDLLTTLINKFDNIQSFLKVMPLIGVNAKFVEEYAFFKDSAKIADELRLIKSFARMGEPIADARRAMYIDAIRILGTNLSYDELKALADTFSLDENGNKLKKGKHGMRNFIINNVISNKRFHYLIRYGDPAHLHEIAKNEAVVKFVLGRIADIQKKQGQNGKNQIDRYYETCIGKDKGKSVSEKVDALTKIITGMNYDQFDKKRSVIEDTGRENAEREKFKKIISLYLTVIYHILKNIVNINARYVIGFHCVERDAQLYKEKGYDINLKKLEEKGFSSVTKLCAGIDETAPDKRKDVEKEMAERAKESIDSLESANPKLYANYIKYSDEKKAEEFTRQINREKAKTALNAYLRNTKWNVIIREDLLRIDNKTCTLFRNKAVHLEVARYVHAYINDIAEVNSYFQLYHYIMQRIIMNERYEKSSGKVSEYFDAVNDEKKYNDRLLKLLCVPFGYCIPRFKNLSIEALFDRNEAAKFDKEKKKVSGNSTSAPKKKRKVGGSPAAKRVKLDGSDYKDHDGDYKDHDIDYKDDDDK

SEQ ID NO: 4 - DNA, 474 bp, synthetic construct
  source 1..474: mol_type = other DNA
atatcactgatcgctgcactcgcggttgattatgtaataggcatggaaaacgcgatgccctggaacctgccagcagacctcgcatggtttaagcggaacactctgaataagccagtcattatgggccgacatacgtgggaaagcattggacgcccactgccaggaaggaagaatataatcctgtcatcacagccgtccacggacgatagagtcacttgggtaaaatccgttgacgaagccatagctgcctgtggtgatgtgcctgagataatggtaataggaggagggcgggtaatcgaacaatttctgcctaaagcccagaagctgtaccttacccatattgacgcggaagtagagggcgacacacatttcccagattatgagccagatgattgggagagcgttttctccgaatttcatgatgcagacgcccagaatagccacagctactgctttgaaatattggagcgccga

SEQ ID NO: 5 - protein, 158 aa, synthetic construct
ISLIAALAVDYVIGMENAMPWNLPADLAWFKRNTLNKPVIMGRHTWESIGRPLPGRKNIILSSQPSTDDRVTWVKSVDEAIAACGDVPEIMVIGGGRVIEQFLPKAQKLYLTHIDAEVEGDTHFPDYEPDDWESVFSEFHDADAQNSHSYCFEILERR

SEQ ID NO: 6 - DNA, 3522 bp, synthetic construct
  source 1..3522: mol_type = other DNA
atgatcgagaaaaagaagtccttcgctaagggcatgggcgtgaagtccacactcgtgtccggctccaaagtgtacatgacaaccttcgccgaaggcagcgacgccaggctggaaaagatcgtggagggcgacagcatcaggagcgtgaatgagggcgaggccttcagcgctgaaatggccgataaaaacgccggctataagatcggcaacgccaaattcagccatcctaagggctacgccgtggtggctaacaaccctctgtatacaggacccgtccagcaggatatgctcggcctgaaggaaactctggaaaagaggtacttcggcgagagcgctgatggcaatgacaatatttgtatccaggtgatccataacatcctggacattgaaaaaatcctcgccgaatacattaccaacgccgcctacgccgtcaacaatatctccggcctggataaggacattattggattcggcaagttctccacagtgtatacctacgacgaattcaaagaccccgagcaccatagggccgctttcaacaataacgataagctcatcaacgccatcaaggcccagtatgacgagttcgacaacttcctcgataaccccagactcggctatttcggccaggcctttttcagcaaggagggcagaaattacatcatcaattacggcaacgaatgctatgacattctggccctcctgagcggactgagacactgggtggtccacaacaacgaagaagagtccaggatctccaggacctggctctacaacctcgataagaacctcgacaacgaatacatctccaccctcaactacctctacgacaggatcaccaatgagctgaccaactccttctccaagaactccgccgccaacgtgaactatattgccgaaactctgggaatcaaccctgccgaattcgccgaacaatatttcagattcagcattatgaaagagcagaaaaacctcggattcaatatcaccaagctcagggaagtgatgctggacaggaaggatatgtccgagatcaggaaaaatcataaggtgttcgactccatcaggaccaaggtctacaccatgatggactttgtgatttataggtattacatcgaagaggatgccaaggtggctgccgccaataagtccctccccgataatgagaagtccctgagcgagaaggatatctttgtgattaacctgaggggctccttcaacgacgaccagaaggatgccctctactacgatgaagctaatagaatttggagaaagctcgaaaatatcatgcacaacatcaaggaatttaggggaaacaagacaagagagtataagaagaaggacgcccctagactgcccagaatcctgcccgctggccgtgatgtttccgccttcagcaaactcatgtatgccctgacaatgttcctggatggcaaggagatcaacgacctcctgaccaccctgattaataaattcgataacatccagagcttcctgaaggtgatgcctctcatcggagtcaacgctaagttcgtggaggaatacgcctttttcaaagactccgccaagatcgccgatgagctgaggctgatcaagtccttcgctagaatgggagaacctattgccgatgccaggagggccatgtatatcgacgccatccgtattttaggaaccaacctgtcctatgatgagctcaaggccctcgccgacaccttttccctggacgagaacggaaacaagctcaagaaaggcaagcacggcatgagaaatttcattattaataacgtgatcagcaataaaaggttccactacctgatcagatacggtgatcctgcccacctccatgagatcgccaaaaacgaggccgtggtgaagttcgtgctcggcaggatcgctgacatccagaaaaaacagggccagaacggcaagaaccagatcgacaggtactacgaaacttgtatcggaaaggataagggcaagagcgtgagcgaaaaggtggacgctctcacaaagatcatcaccggaatgaactacgaccaattcgacaagaaaaggagcgtcattgaggacaccggcagggaaaacgccgagagggagaagtttaaaaagatcatcagcctgtacctcaccgtgatctaccacatcctcaagaatattgtcaatatcaacgccaggtacgtcatcggattccattgcgtcgagcgtgatgctcaactgtacaaggagaaaggctacgacatcaatctcaagaaactggaagagaagggattcagctccgtcaccaagctctgcgctggcattgatgaaactgcccccgataagagaaaggacgtggaaaaggagatggctgaaagagccaaggagagcattgacagcctcgagagcgccaaccccaagctgtatgccaattacatcaaatacagcgacgagaagaaagccgaggagttcaccaggcagattaacagggagaaggccaaaaccgccctgaacgcctacctgaggaacaccaagtggaatgtgatcatcagggaggacctcctgagaattgacaacaagacatgtaccctgttcaggaacaaggccgtccatctggaagtggccaggtatgtccacgcctatatcaacgacattgccgaggtcaattcctacttccaactgtaccattacatcatgcagagaattatcatgaatgagaggtacgagaaaagcagcggaaaggtgtccgagtacttcgacgctgtgaatgacgagaagaagtacaacgataggctcctgaaactgctgtgtgtgcctttcggctactgtatccccaggtttaagaacctgagcatcgaggccctgttcgataggaacgaggccgccaagttcgacaaggagaaaaagaaagtgtccggcaattccactagtgctcccaagaaaaagcgcaaggtaggtggaagcccagcagctaaaagagttaaattggatggatccatatcactgatcgctgcactcgcggttgattatgtaataggcatggaaaacgcgatgccctggaacctgccagcagacctcgcatggtttaagcggaacactctgaataagccagtcattatgggccgacatacgtgggaaagcattggacgcccactgccaggaaggaagaatataatcctgtcatcacagccgtccacggacgatagagtcacttgggtaaaatccgttgacgaagccatagctgcctgtggtgatgtgcctgagataatggtaataggaggagggcgggtaatcgaacaatttctgcctaaagcccagaagctgtaccttacccatattgacgcggaagtagagggcgacacacatttcccagattatgagccagatgattgggagagcgttttctccgaatttcatgatgcagacgcccagaatagccacagctactgctttgaaatattggagcgccgaggatccgattataaggatcacgatggagattacaaggaccacgacatagactacaaagatgatgacgacaagtaa

SEQ ID NO: 7 - protein, 1173 aa, synthetic construct
MIEKKKSFAKGMGVKSTLVSGSKVYMTTFAEGSDARLEKIVEGDSIRSVNEGEAFSAEMADKNAGYKIGNAKFSHPKGYAVVANNPLYTGPVQQDMLGLKETLEKRYFGESADGNDNICIQVIHNILDIEKILAEYITNAAYAVNNISGLDKDIIGFGKFSTVYTYDEFKDPEHHRAAFNNNDKLINAIKAQYDEFDNFLDNPRLGYFGQAFFSKEGRNYIINYGNECYDILALLSGLRHWVVHNNEEESRISRTWLYNLDKNLDNEYISTLNYLYDRITNELTNSFSKNSAANVNYIAETLGINPAEFAEQYFRFSIMKEQKNLGFNITKLREVMLDRKDMSEIRKNHKVFDSIRTKVYTMMDFVIYRYYIEEDAKVAAANKSLPDNEKSLSEKDIFVINLRGSFNDDQKDALYYDEANRIWRKLENIMHNIKEFRGNKTREYKKKDAPRLPRILPAGRDVSAFSKLMYALTMFLDGKEINDLLTTLINKFDNIQSFLKVMPLIGVNAKFVEEYAFFKDSAKIADELRLIKSFARMGEPIADARRAMYIDAIRILGTNLSYDELKALADTFSLDENGNKLKKGKHGMRNFIINNVISNKRFHYLIRYGDPAHLHEIAKNEAVVKFVLGRIADIQKKQGQNGKNQIDRYYETCIGKDKGKSVSEKVDALTKIITGMNYDQFDKKRSVIEDTGRENAEREKFKKIISLYLTVIYHILKNIVNINARYVIGFHCVERDAQLYKEKGYDINLKKLEEKGFSSVTKLCAGIDETAPDKRKDVEKEMAERAKESIDSLESANPKLYANYIKYSDEKKAEEFTRQINREKAKTALNAYLRNTKWNVIIREDLLRIDNKTCTLFRNKAVHLEVARYVHAYINDIAEVNSYFQLYHYIMQRIIMNERYEKSSGKVSEYFDAVNDEKKYNDRLLKLLCVPFGYCIPRFKNLSIEALFDRNEAAKFDKEKKKVSGNSTSAPKKKRKVGGSPAAKRVKLDGSISLIAALAVDYVIGMENAMPWNLPADLAWFKRNTLNKPVIMGRHTWESIGRPLPGRKNIILSSQPSTDDRVTWVKSVDEAIAACGDVPEIMVIGGGRVIEQFLPKAQKLYLTHIDAEVEGDTHFPDYEPDDWESVFSEFHDADAQNSHSYCFEILERRGSDYKDHDGDYKDHDIDYKDDDDK

SEQ ID NO: 8 - DNA, 584 bp, synthetic construct
  source 1..584: mol_type = other DNA
aacccctaccaactggtcggggtttgaaacgagcagttcaaaatgacccagtccaagtaaacccctaccaactggtcggggtttgaaacagcaacagacgtaagaaccagagcaagtaaacccctaccaactggtcggggtttgaaacaaaccaaaggaacatgaccagcccaagtaaacccctaccaactggtcggggtttgaaacatagttgatagtaatccagccatcaagtaaacccctaccaactggtcggggtttgaaacagacaattgtgtcgctgccatcgcaagtaaacccctaccaactggtcggggtttgaaacgcaaagtcagatttgttgctccacaagtaaacccctaccaactggtcggggtttgaaacccacaaaagctagaggaagccagcaagtaaacccctaccaactggtcggggtttgaaacccatcatgtaggttgccgcacagcaagtaaacccctaccaactggtcggggtttgaaacggtaccagtttagcacgaagctccaagtaaacccctaccaactggtcggggtttgaaaccagaagaaaagtcagaggacacc

SEQ ID NO: 9 - DNA, 30 bp, synthetic construct
  source 1..30: mol_type = other DNA
aacccctaccaactggtcggggtttgaaac

SEQ ID NO: 10 - DNA, 36 bp, synthetic construct
  source 1..36: mol_type = other DNA
caagtaaacccctaccaactggtcggggtttgaaac

SEQ ID NO: 11 - protein, 20 aa, synthetic construct
APKKKRKVGGSPAAKRVKLD

SEQ ID NO: 12 - RNA, 23 nt, synthetic construct
  source 1..23: mol_type = other RNA
cgaagataagagccagagccagc

SEQ ID NO: 13 - RNA, 23 nt, synthetic construct
  source 1..23: mol_type = other RNA
ctttcatcagtcctgagcaccac

SEQ ID NO: 14 - RNA, 23 nt, synthetic construct
  source 1..23: mol_type = other RNA
cagaagaaaagtcagaggacacc

SEQ ID NO: 15 - RNA, 23 nt, synthetic construct
  source 1..23: mol_type = other RNA
gagcagttcaaaatgacccagtc

SEQ ID NO: 16 - RNA, 23 nt, synthetic construct
  source 1..23: mol_type = other RNA
gtcgccattgtctccagtcacca

SEQ ID NO: 17 - RNA, 23 nt, synthetic construct
  source 1..23: mol_type = other RNA
agaaacagcaagcccaggaactg

SEQ ID NO: 18 - RNA, 23 nt, synthetic construct
  source 1..23: mol_type = other RNA
agtccacagagaacacaggcacg

SEQ ID NO: 19 - RNA, 23 nt, synthetic construct
  source 1..23: mol_type = other RNA
ggtaccagtttagcacgaagctc

SEQ ID NO: 20 - RNA, 23 nt, synthetic construct
  source 1..23: mol_type = other RNA
gaaagacaatggtggcatactcc

SEQ ID NO: 21 - RNA, 22 nt, synthetic construct
  source 1..22: mol_type = other RNA
gtaactactcgagacgcctatt

SEQ ID NO: 22 - RNA, 23 nt, synthetic construct
  source 1..23: mol_type = other RNA
agcaacagacgtaagaaccagag

SEQ ID NO: 23 - RNA, 23 nt, synthetic construct
  source 1..23: mol_type = other RNA
ccatcatgtaggttgccgcacag

SEQ ID NO: 24 - RNA, 23 nt, synthetic construct
  source 1..23: mol_type = other RNA
aaaccaaaggaacatgaccagcc

SEQ ID NO: 25 - RNA, 23 nt, synthetic construct
  source 1..23: mol_type = other RNA
atagttgatagtaatccagccat

SEQ ID NO: 26 - RNA, 23 nt, synthetic construct
  source 1..23: mol_type = other RNA
agacaattgtgtcgctgccatcg

SEQ ID NO: 27 - RNA, 23 nt, synthetic construct
  source 1..23: mol_type = other RNA
gcaaagtcagatttgttgctcca

SEQ ID NO: 28 - RNA, 23 nt, synthetic construct
  source 1..23: mol_type = other RNA
ccacaaaagctagaggaagccag

SEQ ID NO: 29 - RNA, 23 nt, synthetic construct
  source 1..23: mol_type = other RNA
accattatcacaagaccgaacag

SEQ ID NO: 30 - RNA, 23 nt, synthetic construct
  source 1..23: mol_type = other RNA
aagaataacgcataggcagacac

SEQ ID NO: 31 - RNA, 23 nt, synthetic construct
  source 1..23: mol_type = other RNA
agaataacgcataggcagacaca

SEQ ID NO: 32 - RNA, 23 nt, synthetic construct
  source 1..23: mol_type = other RNA
caattttagagacttcgccaaag

SEQ ID NO: 33 - RNA, 23 nt, synthetic construct
  source 1..23: mol_type = other RNA
acgattttggacacgtcaccgaa

SEQ ID NO: 34 - RNA, 23 nt, synthetic construct
  source 1..23: mol_type = other RNA
agatcttgaaatgcacttccgac

SEQ ID NO: 35 - RNA, 23 nt, synthetic construct
  source 1..23: mol_type = other RNA
tcaccatcaaacttgccgccgtg

SEQ ID NO: 36 - RNA, 23 nt, synthetic construct
  source 1..23: mol_type = other RNA
ccatagtagtcagagccactgga

SEQ ID NO: 37 - RNA, 23 nt, synthetic construct
  source 1..23: mol_type = other RNA
acatcgacaagcaagctgtgcag

SEQ ID NO: 38 - RNA, 23 nt, synthetic construct
  source 1..23: mol_type = other RNA
cccaacagacgtgacaggcagct

SEQ ID NO: 39 - RNA, 23 nt, synthetic construct
  source 1..23: mol_type = other RNA
gaaccacttctttgaccatccca

SEQ ID NO: 40 - RNA, 23 nt, synthetic construct
  source 1..23: mol_type = other RNA
aaccacttctttgaccatcccaa

SEQ ID NO: 41 - RNA, 23 nt, synthetic construct
  source 1..23: mol_type = other RNA
agctgattcaaaaagcaggtctt

SEQ ID NO: 42 - RNA, 23 nt, synthetic construct
  source 1..23: mol_type = other RNA
aagtctttaatagagtcgagcca

SEQ ID NO: 43 - RNA, 23 nt, synthetic construct
  source 1..23: mol_type = other RNA
aaaagtctttaatagagtcgagc

SEQ ID NO: 44 - RNA, 23 nt, synthetic construct
  source 1..23: mol_type = other RNA
agaattgttgcacatgctcagca

SEQ ID NO: 45 - RNA, 23 nt, synthetic construct
  source 1..23: mol_type = other RNA
aaatgaaatctagtgccacacag

SEQ ID NO: 46 - RNA, 23 nt, synthetic construct
  source 1..23: mol_type = other RNA
gaacagaattcttcaccagacgc

SEQ ID NO: 47 - RNA, 23 nt, synthetic construct
  source 1..23: mol_type = other RNA
aaattattgagaacagaagcagc

SEQ ID NO: 48 - RNA, 23 nt, synthetic construct
  source 1..23: mol_type = other RNA
gcacaagcataaaaggttcctgt

SEQ ID NO: 49 - RNA, 23 nt, synthetic construct
  source 1..23: mol_type = other RNA
aataatatagttgtctggctagc

SEQ ID NO: 50 - RNA, 23 nt, synthetic construct
  source 1..23: mol_type = other RNA
ctccaaagcatagagtcacctgg

SEQ ID NO: 51 - RNA, 23 nt, synthetic construct
  source 1..23: mol_type = other RNA
accagaaaagtagctgccgccac

SEQ ID NO: 52 - RNA, 23 nt, synthetic construct
  source 1..23: mol_type = other RNA
ccagaaaagtagctgccgccacc

SEQ ID NO: 53 - RNA, 23 nt, synthetic construct
  source 1..23: mol_type = other RNA
tgttccattttagtgcacatccg

SEQ ID NO: 54 - RNA, 23 nt, synthetic construct
  source 1..23: mol_type = other RNA
acaaagttcatagggcagagcag

SEQ ID NO: 55 - RNA, 23 nt, synthetic construct
  source 1..23: mol_type = other RNA
caaagttcatagggcagagcagc

SEQ ID NO: 56 - RNA, 23 nt, synthetic construct
  source 1..23: mol_type = other RNA
ggcacaaagttcatagggcagag

SEQ ID NO: 57 - RNA, 23 nt, synthetic construct
  source 1..23: mol_type = other RNA
gatttctttaagcagcacaccat

SEQ ID NO: 58 - RNA, 23 nt, synthetic construct
  source 1..23: mol_type = other RNA
ttcatagagaaactgaggcacct

SEQ ID NO: 59 - RNA, 23 nt, synthetic construct
  source 1..23: mol_type = other RNA
accgcagatgaatacagcctgta

SEQ ID NO: 60 - RNA, 23 nt, synthetic construct
  source 1..23: mol_type = other RNA
agaattcatgttattgccccaag

SEQ ID NO: 61 - RNA, 23 nt, synthetic construct
  source 1..23: mol_type = other RNA
cagaattcatgttattgccccaa

SEQ ID NO: 62 - RNA, 23 nt, synthetic construct
  source 1..23: mol_type = other RNA
tcatagtaataacggagggcccg

SEQ ID NO: 63 - RNA, 23 nt, synthetic construct
  source 1..23: mol_type = other RNA
aatctgtcgtcatagcacaccac

SEQ ID NO: 64 - RNA, 23 nt, synthetic construct
  source 1..23: mol_type = other RNA
tccagaaaccagttcactgccag

SEQ ID NO: 65 - RNA, 23 nt, synthetic construct
  source 1..23: mol_type = other RNA
cacacagcttagtatacacgctg

SEQ ID NO: 66 - RNA, 23 nt, synthetic construct
  source 1..23: mol_type = other RNA
accatcctacagaaggggccagc

SEQ ID NO: 67 - RNA, 23 nt, synthetic construct
  source 1..23: mol_type = other RNA
actgcattgtcggctgccacctg

SEQ ID NO: 68 - RNA, 23 nt, synthetic construct
  source 1..23: mol_type = other RNA
cagtagaatccgcaggcgtccag

SEQ ID NO: 69 - RNA, 23 nt, synthetic construct
  source 1..23: mol_type = other RNA
atcatccaagagttggtcatcct

SEQ ID NO: 70 - RNA, 23 nt, synthetic construct
  source 1..23: mol_type = other RNA
atgcgcttcagttcgaacaccgt

SEQ ID NO: 71 - RNA, 23 nt, synthetic construct
  source 1..23: mol_type = other RNA
ttggtcatccttgtacagccgct

SEQ ID NO: 72 - RNA, 23 nt, synthetic construct
  source 1..23: mol_type = other RNA
ccacagagaacacaggcacggct

SEQ ID NO: 73 - RNA, 23 nt, synthetic construct
  source 1..23: mol_type = other RNA
accatttgaaaattaaagcgccg

SEQ ID NO: 74 - RNA, 23 nt, synthetic construct
  source 1..23: mol_type = other RNA
ccgaagataagagccagagccag

SEQ ID NO: 75 - RNA, 23 nt, synthetic construct
  source 1..23: mol_type = other RNA
accttgtaagtagtagcagcagc

SEQ ID NO: 76 - RNA, 23 nt, synthetic construct
  source 1..23: mol_type = other RNA
cagttcaaaatgacccagtcgga

SEQ ID NO: 77 - RNA, 23 nt, synthetic construct
  source 1..23: mol_type = other RNA
gcagaaaatcgtcttggtcgcca

SEQ ID NO: 78 - RNA, 23 nt, synthetic construct
  source 1..23: mol_type = other RNA
cgctgaaatccaaggcaagccat

SEQ ID NO: 79 - RNA, 23 nt, synthetic construct
  source 1..23: mol_type = other RNA
aaaaacaggagagtgcagggcca

SEQ ID NO: 80 - RNA, 23 nt, synthetic construct
  source 1..23: mol_type = other RNA
acattgtaagatgatagagccac

SEQ ID NO: 81 - RNA, 23 nt, synthetic construct
  source 1..23: mol_type = other RNA
gacattgaagtagtcatgcagct

SEQ ID NO: 82 - RNA, 23 nt, synthetic construct
  source 1..23: mol_type = other RNA
tctttctagtcaacgcgaccacc

SEQ ID NO: 83 - RNA, 23 nt, synthetic construct
  source 1..23: mol_type = other RNA
cagataaatttattgccactgtt

SEQ ID NO: 84 - RNA, 23 nt, synthetic construct
  source 1..23: mol_type = other RNA
tagcaacagacgtaagaaccaga

SEQ ID NO: 85 - RNA, 23 nt, synthetic construct
  source 1..23: mol_type = other RNA
aaaatcggtgaataggcagacag

SEQ ID NO: 86 - RNA, 23 nt, synthetic construct
  source 1..23: mol_type = other RNA
tcatgagcagattaaacccggcc

SEQ ID NO: 87 - RNA, 23 nt, synthetic construct
  source 1..23: mol_type = other RNA
aagagaatttgctaacggaccag

SEQ ID NO: 88 - RNA, 23 nt, synthetic construct
  source 1..23: mol_type = other RNA
ttaaatatatgcttaagtcaggg

SEQ ID NO: 89 - RNA, 23 nt, synthetic construct
  source 1..23: mol_type = other RNA
atatttaatcgaagagaccccag

SEQ ID NO: 90 - RNA, 23 nt, synthetic construct
  source 1..23: mol_type = other RNA
cactcaaaatgtaagcagctgag

SEQ ID NO: 91 - RNA, 23 nt, synthetic construct
  source 1..23: mol_type = other RNA
tagactgcaatagttccacaagc

SEQ ID NO: 92 - RNA, 23 nt, synthetic construct
  source 1..23: mol_type = other RNA
ccagccaacaaaagcgccaacca

SEQ ID NO: 93 - RNA, 23 nt, synthetic construct
  source 1..23: mol_type = other RNA
aaaacaagacaagtgtctaccag

SEQ ID NO: 94 - RNA, 23 nt, synthetic construct
  source 1..23: mol_type = other RNA
agaaattgagaaatgaagctggt

SEQ ID NO: 95 - RNA, 23 nt, synthetic construct
  source 1..23: mol_type = other RNA
tcaccagaagcgtaccatactcg

SEQ ID NO: 96 - RNA, 23 nt, synthetic construct
  source 1..23: mol_type = other RNA
taacccgatgagctacccagtag

SEQ ID NO: 97 - RNA, 23 nt, synthetic construct
  source 1..23: mol_type = other RNA
cgctaatgcactcaatcccgagg

SEQ ID NO: 98 - RNA, 23 nt, synthetic construct
  source 1..23: mol_type = other RNA
cctgacgcgacatatcagctaag

SEQ ID NO: 99 - RNA, 23 nt, synthetic construct
  source 1..23: mol_type = other RNA
agtaggccgggggtgtagacctt

SEQ ID NO: 100 - RNA, 23 nt, synthetic construct
  source 1..23: mol_type = other RNA
ctaatcggcttcaacgtgccgca

SEQ ID NO: 101 - RNA, 23 nt, synthetic construct
  source 1..23: mol_type = other RNA
ctattgaggcattgactgatgcc

SEQ ID NO: 102 - RNA, 23 nt, synthetic construct
  source 1..23: mol_type = other RNA
aggacatatgccctacctccatg

SEQ ID NO: 103 - DNA, 154 bp, synthetic construct
  source 1..154: mol_type = other DNA
tcttgtggaaaggacgaaacaccgaacccctaccaactggtcggggtttgaaacgaaagacaatggtggcatactccaacccctaccaactggtcggggtttgaaacaataatatagttgtctggctagctttttttgaattcgctagctaggt

SEQ ID NO: 104 - DNA, 136 bp, synthetic construct
  source 1..136: mol_type = other DNA
tcttgtggaaaggacgaaacaccgaacccctaccaactggtcggggtttgaaacgagcagttcaaaatgacccagtccaagtaaacccctaccaactggtcggggtttgaaacagcaacagacgtaagaaccagag

SEQ ID NO: 105 - DNA, 82 bp, synthetic construct
  source 1..82: mol_type = other DNA
ctgtgcggcaacctacatgatgggtttcaaaccccgaccagttggtaggggtttacttgctctggttcttacgtctgttgct

SEQ ID NO: 106 - DNA, 165 bp, synthetic construct
  source 1..165: mol_type = other DNA
ccatcatgtaggttgccgcacagcaagtaaacccctaccaactggtcggggtttgaaacggtaccagtttagcacgaagctccaagtaaacccctaccaactggtcggggtttgaaaccagaagaaaagtcagaggacacctttttttgaattcgctagctaggt

SEQ ID NO: 107 - DNA, 141 bp, synthetic construct
  source 1..141: mol_type = other DNA
atggctggattactatcaactatgtttcaaaccccgaccagttggtaggggtttacttgggctggtcatgttcctttggtttgtttcaaaccccgaccagttggtaggggtttacttgctctggttcttacgtctgttgct

SEQ ID NO: 108 - DNA, 142 bp, synthetic construct
  source 1..142: mol_type = other DNA
atagttgatagtaatccagccatcaagtaaacccctaccaactggtcggggtttgaaaccagacaattgtgtcgctgccatcgcaagtaaacccctaccaactggtcggggtttgaaacgcaaagtcagatttgttgctcca

SEQ ID NO: 109 - DNA, 141 bp, synthetic construct
  source 1..141: mol_type = other DNA
ctgtgcggcaacctacatgatgggtttcaaaccccgaccagttggtaggggtttacttgctggcttcctctagcttttgtgggtttcaaaccccgaccagttggtaggggtttacttgtggagcaacaaatctgactttgc